SEQUENCE LISTING (96 sequences)

SEQ ID NO: 1 - protein, 121 aa, Artificial Sequence
  VHH1
DVQLQESGGGLVQPGGSLRLSCATSGFTLDDYTIGWVRQAPGKEHEGVSCFSPSDGTTYYADSVKGRFTFSRDNAKNTVYLQMNSLKPEDTAVYYCAASFADGSSWCYDYWGQGTQVTVSS

SEQ ID NO: 2 - protein, 123 aa, Artificial Sequence
  VHH2
DVQLQESGGGLVQAGGSLRLSCAVSGQTWTNYHIGWFRQAPGKARESVASIEWGGRGTYATDSVKGRFTISRDNAKNTVYLQMNSLKPEDTAVYYCAAQSSSRSPLESNYDYWGQGTQVTVSS

SEQ ID NO: 3 - protein, 126 aa, Artificial Sequence
  VHH3
DVQLQESGGGLVQPGESLKLSCLVSGVTYHFYDITWFRQAPGLEREGVSCISYASGTTKFADSVKGRFTISRDNAKNAVYLEMNSLKPEDTAVYFCATQGSGSCSDFMSAGSVLSWGQGTQVTVSS

SEQ ID NO: 4 - protein, 126 aa, Artificial Sequence
  VHH4
DVQLQESGGGLVQPGESLRLSCLVSGVTYHFYDITWFRQAPGLEREGVSCISYASGSTKFADSVKGRFTISRDNAKNAVYLQMNSLKPEDTAVYFCAMQGSGSCSDFMSADSVLSWGQGTQVTVSS

SEQ ID NO: 5 - protein, 126 aa, Artificial Sequence
  VHH5
DVQLQESGGGLVQPGESLRLSCLVSGVTYHFYDITWFRQAPGLEREGVSCISYASGSTKFADSVKGRFTISRDNAKNAVYLQMNSLKPEDTAVYFCATQGSGSCSDFMSADSVLSWGQGTQVTVSS

SEQ ID NO: 6 - protein, 122 aa, Artificial Sequence
  VHH6
DVQLQESGGGLVQTGGSLRLSCAASGLPFSKYAIAWFRQAPGKESEFLVVVSWSGDKFYQDNMKGRCTISRDNAKNTAYLQINSLQPEDTAVYYCAASGSYPVTDRYFYNFWGQGTQVTVSS

SEQ ID NO: 7 - protein, 129 aa, Artificial Sequence
  VHH7
DVQLQESGGGLAQPGGSLRLSCAASGFTFSSYDMGWYRQVPGKECELVAAIFSAGGSTIYADSVKGRFTISRDNAKNTVFLQMNSLKPEDTAVYYCNKAPPFTTIQPTCVMIDTQLGAWGQGTQVTVSS

SEQ ID NO: 8 - protein, 129 aa, Artificial Sequence
  VHH8
DVQLQESGGGLAQPGGSLRLSCAASGFTFSSYDMGWYRQAPGKECELVAAIFSADGSTIYADSVKGRFTISSDNAKNTVFLQMNSLKPEDTAVYYCNKAPPFTTIQPMCVMIDTQLGAWGQGTQVTVSS

SEQ ID NO: 9 - protein, 129 aa, Artificial Sequence
  VHH9
DVQLQESGGGLVQAGGSLRLSCAASGRGGASYAMGWFRQAPGKEREFVAAISWTGDTTYNVDSVKGRFTISSDNAKNPVYLDMSSLKPEDTAVYYCAAAHWTRVVAKGAMADEKKYEYWGQGTQVTVSS

SEQ ID NO: 10 - protein, 123 aa, Artificial Sequence
  VHH10
DVQLQESGGGSVQAGGSLRLSCAASGRTFSSNFMGWFRQAPNKDREFVAAIHWSGGSRAYADSVKGRFTISRDNTKDTVYLLVNSLKLEDTAVYYCAAEISWQTTGWTNLGSWGQGTQVTVSS

SEQ ID NO: 11 - protein, 123 aa, Artificial Sequence
  VHH11
DVQLQESGGGLVQAGGSLRLSCAASGRTFSSDFMGWFRQAPGKEREFVAAIHWSGGSTSYADSVKGRFTISRDNAKDTVYLLMNSLKLEDTAVYYCAAEIRWLTTGWANLGSWGQGTQVTVSS

SEQ ID NO: 12 - protein, 123 aa, Artificial Sequence
  VHH12
DVQLQESGGGLVQAGGSLRLSCAASGRTFSSDFMGWFRQAPGKEREFVAAIHWSGGSRSYADSVKGRFTISRDNAKDTVYLLMNSLKLEDTAVYYCAAEIRWQTTGWANLGSWGQGTQVTVSS

SEQ ID NO: 13 - protein, 123 aa, Artificial Sequence
  VHH13
DVQLQESGGGLAQPGGPLRLSCAASGRTFTSDFMGWFRQAPGKEREFVAAIHWSGGSRSYADSVKGRFTISRDNAKDTVYLLMNSLKLEDTAVYYCAAEIRWQTAGWVNLGSWGQGTQVTVSS

SEQ ID NO: 14 - protein, 111 aa, Artificial Sequence
  VHH14
DVQLQESGGGLVQPGGSLRLSCEASGVTFNFYSMTWYRQAPGKERELVADITSEGRTEYAHSVKGRFIISRDDFGTMVYLQMNSLKPEDTAVYYCYTGGSWGQGTQVTVSS

SEQ ID NO: 15 - protein, 116 aa, Artificial Sequence
  VHH15
DVQLQESGGGLVQAGGSLRLSCAVSGSTFRLNVMGWYRQAPGKLRDLVAGITADGGVTNYADSVKGRFIISRDNAKNTVYLQMNSLKPEDTAIYYCNTAPANWGSWGQGTQVTVSS

SEQ ID NO: 16 - protein, 119 aa, Artificial Sequence
  VHH16
DVQLQESGGGLVQPGGSLRLSCTASGSIFSSLTMAWWRQAPGKQRELVAGITTDGSTDYADSVKGRFTISRDNAKNTVYLQMDSLKPEDTAVYYCNKSPYYDPTDYDSWGQGTQVTVSS

SEQ ID NO: 17 - protein, 454 aa, Homo sapiens
  Human B7-H6
MTWRAAASTCAALLILLWALTTEGDLKVEMMAGGTQITPLNDNVTIFCNIFYSQPLNITSMGITWFWKSLTFDKEVKVFEFFGDHQEAFRPGAIVSPWRLKSGDASLRLPGIQLEEAGEYRCEVVVTPLKAQGTVQLEVVASPASRLLLDQVGMKENEDKYMCESSGFYPEAINITWEKQTQKFPHPIEISEDVITGPTIKNMDGTFNVTSCLKLNSSQEDPGTVYQCVVRHASLHTPLRSNFTLTAARHSLSETEKTDNFSIHWWPISFIGVGLVLLIVLIPWKKICNKSSSAYTPLKCILKHWNSFDTQTLKKEHLIFFCTRAWPSYQLQDGEAWPPEGSVNINTIQQLDVFCRQEGKWSEVPYVQAFFALRDNPDLCQCCRIDPALLTVTSGKSIDDNSTKSEKQTPREHSDAVPDAPILPVSPIWEPPPATTSTTPVLSSQPPTLLLPLQ

SEQ ID NO: 18 - protein, 13 aa, Artificial Sequence
  CDR1 of VHH1
ATSGFTLDDYTIG

SEQ ID NO: 19 - protein, 10 aa, Artificial Sequence
  CDR2 of VHH1
CFSPSDGTTY

SEQ ID NO: 20 - protein, 15 aa, Artificial Sequence
  CDR3 of VHH1
AASFADGSSWCYDYW

SEQ ID NO: 21 - protein, 13 aa, Artificial Sequence
  CDR1 of VHH2
AVSGQTWTNYHIG

SEQ ID NO: 22 - protein, 11 aa, Artificial Sequence
  CDR2 of VHH2
SIEWGGRGTYA

SEQ ID NO: 23 - protein, 17 aa, Artificial Sequence
  CDR3 of VHH2
AAQSSSRSPLESNYDYW

SEQ ID NO: 24 - protein, 13 aa, Artificial Sequence
  CDR1 of VHH3
LVSGVTYHFYDIT

SEQ ID NO: 25 - protein, 11 aa, Artificial Sequence
  CDR2 of VHH3
CISYASGTTKF

SEQ ID NO: 26 - protein, 20 aa, Artificial Sequence
  CDR3 of VHH3
ATQGSGSCSDFMSAGSVLSW

SEQ ID NO: 27 - protein, 13 aa, Artificial Sequence
  CDR1 of VHH4
LVSGVTYHFYDIT

SEQ ID NO: 28 - protein, 11 aa, Artificial Sequence
  CDR2 of VHH4
CISYASGSTKF

SEQ ID NO: 29 - protein, 20 aa, Artificial Sequence
  CDR3 of VHH4
AMQGSGSCSDFMSADSVLSW

SEQ ID NO: 30 - protein, 13 aa, Artificial Sequence
  CDR1 of VHH5
LVSGVTYHFYDIT

SEQ ID NO: 31 - protein, 11 aa, Artificial Sequence
  CDR2 of VHH5
CISYASGSTKF

SEQ ID NO: 32 - protein, 20 aa, Artificial Sequence
  CDR3 of VHH5
ATQGSGSCSDFMSADSVLSW

SEQ ID NO: 33 - protein, 13 aa, Artificial Sequence
  CDR1 of VHH6
AASGLPFSKYAIA

SEQ ID NO: 34 - protein, 10 aa, Artificial Sequence
  CDR2 of VHH6
VVSWSGDKFY

SEQ ID NO: 35 - protein, 17 aa, Artificial Sequence
  CDR3 of VHH6
AASGSYPVTDRYFYNFW

SEQ ID NO: 36 - protein, 13 aa, Artificial Sequence
  CDR1 of VHH7
AASGFTFSSYDMG

SEQ ID NO: 37 - protein, 13 aa, Artificial Sequence
  CDR2 of VHH7
AAIFSAGGSTIYA

SEQ ID NO: 38 - protein, 23 aa, Artificial Sequence
  CDR3 of VHH7
NKAPPFTTIQPTCVMIDTQLGAW

SEQ ID NO: 39 - protein, 13 aa, Artificial Sequence
  CDR1 of VHH8
AASGFTFSSYDMG

SEQ ID NO: 40 - protein, 13 aa, Artificial Sequence
  CDR2 of VHH8
AAIFSADGSTIYA

SEQ ID NO: 41 - protein, 23 aa, Artificial Sequence
  CDR3 of VHH8
NKAPPFTTIQPMCVMIDTQLGAW

SEQ ID NO: 42 - protein, 13 aa, Artificial Sequence
  CDR1 of VHH9
AASGRGGASYAMG

SEQ ID NO: 43 - protein, 12 aa, Artificial Sequence
  CDR2 of VHH9
AAISWTGDTTYN

SEQ ID NO: 44 - protein, 23 aa, Artificial Sequence
  CDR3 of VHH9
AAAHWTRVVAKGAMADEKKYEYW

SEQ ID NO: 45 - protein, 13 aa, Artificial Sequence
  CDR1 of VHH10
AASGRTFSSNFMG

SEQ ID NO: 46 - protein, 13 aa, Artificial Sequence
  CDR2 of VHH10
AAIHWSGGSRAYA

SEQ ID NO: 47 - protein, 17 aa, Artificial Sequence
  CDR3 of VHH10
AAEISWQTTGWTNLGSW

SEQ ID NO: 48 - protein, 13 aa, Artificial Sequence
  CDR1 of VHH11
AASGRTFSSDFMG

SEQ ID NO: 49 - protein, 13 aa, Artificial Sequence
  CDR2 of VHH11
AAIHWSGGSTSYA

SEQ ID NO: 50 - protein, 17 aa, Artificial Sequence
  CDR3 of VHH11
AAEIRWLTTGWANLGSW

SEQ ID NO: 51 - protein, 13 aa, Artificial Sequence
  CDR1 of VHH12
AASGRTFSSDFMG

SEQ ID NO: 52 - protein, 13 aa, Artificial Sequence
  CDR2 of VHH12
AAIHWSGGSRSYA

SEQ ID NO: 53 - protein, 17 aa, Artificial Sequence
  CDR3 of VHH12
AAEIRWQTTGWANLGSW

SEQ ID NO: 54 - protein, 13 aa, Artificial Sequence
  CDR1 of VHH13
AASGRTFTSDFMG

SEQ ID NO: 55 - protein, 13 aa, Artificial Sequence
  CDR2 of VHH13
AAIHWSGGSRSYA

SEQ ID NO: 56 - protein, 17 aa, Artificial Sequence
  CDR3 of VHH13
AAEIRWQTAGWVNLGSW

SEQ ID NO: 57 - protein, 13 aa, Artificial Sequence
  CDR1 of VHH14
EASGVTFNFYSMT

SEQ ID NO: 58 - protein, 12 aa, Artificial Sequence
  CDR2 of VHH14
ADITSEGRTEYA

SEQ ID NO: 59 - protein, 6 aa, Artificial Sequence
  CDR3 of VHH14
YTGGSW

SEQ ID NO: 60 - protein, 13 aa, Artificial Sequence
  CDR1 of VHH15
AVSGSTFRLNVMG

SEQ ID NO: 61 - protein, 13 aa, Artificial Sequence
  CDR2 of VHH15
AGITADGGVTNYA

SEQ ID NO: 62 - protein, 10 aa, Artificial Sequence
  CDR3 of VHH15
NTAPANWGSW

SEQ ID NO: 63 - protein, 14 aa, Artificial Sequence
  CDR1 of VHH16
TASGSIFSSLTMAW

SEQ ID NO: 64 - protein, 12 aa, Artificial Sequence
  CDR2 of VHH16
AGITTDGSTDYA

SEQ ID NO: 65 - protein, 14 aa, Artificial Sequence
  CDR3 of VHH16
NKSPYYDPTDYDSW

SEQ ID NO: 66 - protein, 117 aa, Homo sapiens
  human NKp30 extracellular domain
LWVSQPPEIRTLEGSSAFLPCSFNASQGRLAIGSVTWFRDEVVPGKEVRNGTPEFRGRLAPLASSRFLHDHQAELHIRDVRGHDASIYVCRVEVLGLGVGTGNGTRLVVEKEHPQLG

SEQ ID NO: 67 - protein, 121 aa, Artificial Sequence
  VHH1.1 (humanized)
EVQLVESGGGVVQPGGSLRLSCATSGFTLDDYTIGWVRQAPGKEHEGVSCFSPSDGTTYYADSVKGRFTFSRDNAKNTVYLQMNSLRAEDTAVYYCAASFADGSSWCYDYWGQGTLVTVSS

SEQ ID NO: 68 - protein, 121 aa, Artificial Sequence
  VHH1.2 (humanized)
EVQLVESGGGVVQPGGSLRLSCATSGFTLDDYTIGWVRQAPGKEHEGVSCFSPSDGTTYYADSVKGRFTFSRDNAKNTLYLQMNSLRAEDTAVYYCAASFADGSSWCYDYWGQGTLVTVSS

SEQ ID NO: 69 - protein, 121 aa, Artificial Sequence
  VHH1.3 (humanized)
EVQLVESGGGVVQPGGSLRLSCAASGFTLDDYTIGWVRQAPGKEHEGVSCFSPSDGTTYYADSVKGRFTISRDNAKNTLYLQMNSLRAEDTAVYYCAASFADGSSWCYDYWGQGTLVTVSS

SEQ ID NO: 70 - protein, 121 aa, Artificial Sequence
  VHH1.4 (humanized)
EVQLVESGGGVVQPGGSLRLSCATSGFTLDDYTIGWVRQAPGKGLEGVSCFSPSDGTTYYADSVKGRFTFSRDNAKNTVYLQMNSLRAEDTAVYYCAASFADGSSWCYDYWGQGTLVTVSS

SEQ ID NO: 71 - protein, 121 aa, Artificial Sequence
  VHH1.5 (humanized)
EVQLVESGGGVVQPGGSLRLSCAASGFTLDDYTIGWVRQAPGKGLEGVSCFSPSDGTTYYADSVKGRFTISRDNAKNTVYLQMNSLRAEDTAVYYCAASFADGSSWCYDYWGQGTLVTVSS

SEQ ID NO: 72 - protein, 121 aa, Artificial Sequence
  VHH1.6 (humanized)
EVQLVESGGGVVQPGGSLRLSCATSGFTLDDYTIGWVRQAPGKGLEGVSCFSPSDGTTYYADSVKGRFTFSRDNAKNTLYLQMNSLRAEDTAVYYCAASFADGSSWCYDYWGQGTLVTVSS

SEQ ID NO: 73 - protein, 121 aa, Artificial Sequence
  VHH1.7 (humanized)
EVQLVESGGGVVQPGGSLRLSCAASGFTLDDYTIGWVRQAPGKGLEGVSCFSPSDGTTYYADSVKGRFTFSRDNAKNTLYLQMNSLRAEDTAVYYCAASFADGSSWCYDYWGQGTLVTVSS

SEQ ID NO: 74 - protein, 121 aa, Artificial Sequence
  VHH1.8 (humanized)
EVQLVESGGGVVQPGGSLRLSCATSGFTLDDYTIGWVRQAPGKGLEGVSCFSPSDGTTYYADSVKGRFTISRDNAKNTLYLQMNSLRAEDTAVYYCAASFADGSSWCYDYWGQGTLVTVSS

SEQ ID NO: 75 - protein, 121 aa, Artificial Sequence
  VHH1.9 (humanized)
EVQLVESGGGVVQPGGSLRLSCAASGFTLDDYTIGWVRQAPGKGLEGVSCFSPSDGTTYYADSVKGRFTISRDNAKNTLYLQMNSLRAEDTAVYYCAASFADGSSWCYDYWGQGTLVTVSS

SEQ ID NO: 76 - protein, 121 aa, Artificial Sequence
  VHH1.10 (humanized)
EVQLVESGGGVVQPGGSLRLSCAASGFTLDDYTIGWVRQAPGKGLEWVSCFSPSDGTTYYADSVKGRFTISRDNAKNTLYLQMNSLRAEDTAVYYCAASFADGSSWCYDYWGQGTLVTVSS

SEQ ID NO: 77 - protein, 121 aa, Artificial Sequence
  VHH1.11 (humanized)
EVQLVESGGGVVQPGGSLRLSCATSGFTLDDYTIGWVRQAPGKEHEGVSCFSPSDGTTYYADSVKGRFTISRDNAKNTLYLQMNSLRAEDTAVYYCAASFADGSSWCYDYWGQGTLVTVSS

SEQ ID NO: 78 - protein, 121 aa, Artificial Sequence
  VHH1.12 (humanized)
EVQLVESGGGVVQPGGSLRLSCAASGFTLDDYTIGWVRQAPGKEHEGVSCFSPSDGTTYYADSVKGRFTFSRDNAKNTLYLQMNSLRAEDTAVYYCAASFADGSSWCYDYWGQGTLVTVSS

SEQ ID NO: 79 - protein, 123 aa, Artificial Sequence
  VHH2.1 (humanized)
EVQLVESGGGVVQAGGSLRLSCAVSGQTWTNYHIGWFRQAPGKARESVASIEWGGRGTYATDSVKGRFTISRDNAKNTVYLQMNSLRAEDTAVYYCAAQSSSRSPLESNYDYWGQGTLVTVSS

SEQ ID NO: 80 - protein, 123 aa, Artificial Sequence
  VHH2.2 (humanized)
EVQLVESGGGVVQPGGSLRLSCAVSGQTWTNYHIGWFRQAPGKARESVASIEWGGRGTYATDSVKGRFTISRDNAKNTVYLQMNSLRAEDTAVYYCAAQSSSRSPLESNYDYWGQGTLVTVSS

SEQ ID NO: 81 - protein, 123 aa, Artificial Sequence
  VHH2.3 (humanized)
EVQLVESGGGVVQPGGSLRLSCAVSGQTWTNYHIGWFRQAPGKARESVASIEWGGRGTYATDSVKGRFTISRDNAKNTLYLQMNSLRAEDTAVYYCAAQSSSRSPLESNYDYWGQGTLVTVSS

SEQ ID NO: 82 - protein, 123 aa, Artificial Sequence
  VHH2.4 (humanized)
EVQLVESGGGVVQPGGSLRLSCAASGQTWTNYHIGWFRQAPGKARESVASIEWGGRGTYATDSVKGRFTISRDNAKNTLYLQMNSLRAEDTAVYYCAAQSSSRSPLESNYDYWGQGTLVTVSS

SEQ ID NO: 83 - protein, 123 aa, Artificial Sequence
  VHH2.5 (humanized)
EVQLVESGGGVVQPGGSLRLSCAVSGQTWTNYHIGWFRQAPGKGLESVASIEWGGRGTYATDSVKGRFTISRDNAKNTVYLQMNSLRAEDTAVYYCAAQSSSRSPLESNYDYWGQGTLVTVSS

SEQ ID NO: 84 - protein, 123 aa, Artificial Sequence
  VHH2.6 (humanized)
EVQLVESGGGVVQPGGSLRLSCAVSGQTWTNYHIGWVRQAPGKGLESVASIEWGGRGTYATDSVKGRFTISRDNAKNTVYLQMNSLRAEDTAVYYCAAQSSSRSPLESNYDYWGQGTLVTVSS

SEQ ID NO: 85 - protein, 123 aa, Artificial Sequence
  VHH2.7 (humanized)
EVQLVESGGGVVQPGGSLRLSCAVSGQTWTNYHIGWFRQAPGKGLESVASIEWGGRGTYATDSVKGRFTISRDNAKNTLYLQMNSLRAEDTAVYYCAAQSSSRSPLESNYDYWGQGTLVTVSS

SEQ ID NO: 86 - protein, 123 aa, Artificial Sequence
  VHH2.8 (humanized)
EVQLVESGGGVVQPGGSLRLSCAVSGQTWTNYHIGWFRQAPGKGLEWVASIEWGGRGTYATDSVKGRFTISRDNAKNTLYLQMNSLRAEDTAVYYCAAQSSSRSPLESNYDYWGQGTLVTVSS

SEQ ID NO: 87 - protein, 123 aa, Artificial Sequence
  VHH2.9 (humanized)
EVQLVESGGGVVQPGGSLRLSCAVSGQTWTNYHIGWVRQAPGKGLESVASIEWGGRGTYATDSVKGRFTISRDNAKNTLYLQMNSLRAEDTAVYYCAAQSSSRSPLESNYDYWGQGTLVTVSS

SEQ ID NO: 88 - protein, 123 aa, Artificial Sequence
  VHH2.10 (humanized)
EVQLVESGGGVVQPGGSLRLSCAVSGQTWTNYHIGWVRQAPGKGLEWVASIEWGGRGTYATDSVKGRFTISRDNAKNTLYLQMNSLRAEDTAVYYCAAQSSSRSPLESNYDYWGQGTLVTVSS

SEQ ID NO: 89 - protein, 123 aa, Artificial Sequence
  VHH2.11 (humanized)
EVQLVESGGGVVQPGGSLRLSCAASGQTWTNYHIGWFRQAPGKGLESVASIEWGGRGTYATDSVKGRFTISRDNAKNTVYLQMNSLRAEDTAVYYCAAQSSSRSPLESNYDYWGQGTLVTVSS

SEQ ID NO: 90 - protein, 123 aa, Artificial Sequence
  VHH2.12 (humanized)
EVQLVESGGGVVQPGGSLRLSCAASGQTWTNYHIGWFRQAPGKGLEWVASIEWGGRGTYATDSVKGRFTISRDNAKNTVYLQMNSLRAEDTAVYYCAAQSSSRSPLESNYDYWGQGTLVTVSS

SEQ ID NO: 91 - protein, 123 aa, Artificial Sequence
  VHH2.13 (humanized)
EVQLVESGGGVVQPGGSLRLSCAASGQTWTNYHIGWVRQAPGKGLESVASIEWGGRGTYATDSVKGRFTISRDNAKNTVYLQMNSLRAEDTAVYYCAAQSSSRSPLESNYDYWGQGTLVTVSS

SEQ ID NO: 92 - protein, 123 aa, Artificial Sequence
  VHH2.14 (humanized)
EVQLVESGGGVVQPGGSLRLSCAASGQTWTNYHIGWVRQAPGKGLEWVASIEWGGRGTYATDSVKGRFTISRDNAKNTVYLQMNSLRAEDTAVYYCAAQSSSRSPLESNYDYWGQGTLVTVSS

SEQ ID NO: 93 - protein, 123 aa, Artificial Sequence
  VHH2.15 (humanized)
EVQLVESGGGVVQPGGSLRLSCAASGQTWTNYHIGWFRQAPGKGLESVASIEWGGRGTYATDSVKGRFTISRDNAKNTLYLQMNSLRAEDTAVYYCAAQSSSRSPLESNYDYWGQGTLVTVSS

SEQ ID NO: 94 - protein, 123 aa, Artificial Sequence
  VHH2.16 (humanized)
EVQLVESGGGVVQPGGSLRLSCAASGQTWTNYHIGWFRQAPGKGLEWVASIEWGGRGTYATDSVKGRFTISRDNAKNTLYLQMNSLRAEDTAVYYCAAQSSSRSPLESNYDYWGQGTLVTVSS

SEQ ID NO: 95 - protein, 123 aa, Artificial Sequence
  VHH2.17 (humanized)
EVQLVESGGGVVQPGGSLRLSCAASGQTWTNYHIGWVRQAPGKGLESVASIEWGGRGTYATDSVKGRFTISRDNAKNTLYLQMNSLRAEDTAVYYCAAQSSSRSPLESNYDYWGQGTLVTVSS

SEQ ID NO: 96 - protein, 123 aa, Artificial Sequence
  VHH2.18 (humanized)
EVQLVESGGGVVQPGGSLRLSCAASGQTWTNYHIGWVRQAPGKGLEWVASIEWGGRGTYATDSVKGRFTISRDNAKNTLYLQMNSLRAEDTAVYYCAAQSSSRSPLESNYDYWGQGTLVTVSS

(19) United States
(12) Patent Application Publication
(10) Pub. No.: US 2024/0287177 A1
(43) Pub. Date: Aug. 29, 2024

(54) VHH-BASED NKP30 BINDERS

(71) Applicant: Merck Patent GmbH, Darmstadt (DE)

(72) Inventors: Stefan ZIELONKA, Darmstadt (DE); Lars Toleikis, Darmstadt (DE); Simon Krah, Darmstadt (DE); Lukas Pekar, Darmstadt (DE); Andreas Evers, Darmstadt (DE)

(73) Assignee: Merck Patent GmbH, Darmstadt (DE)

(21) Appl. No.: 18/572,504

(22) PCT Filed: Jun. 22, 2022
(86) PCT No.: PCT/EP2022/066963

§ 371 Date: Dec. 20, 2023

(30) Foreign Application Priority Data
Jun. 22, 2021 (EP) 21180872.0

(51) Int. Cl. C07K 16/28; A61K 39/00

(52) U.S. Cl. CPC C07K 16/2803; A61K 2039/505; C07K 2317/22; C07K 2317/24; C07K 2317/31; C07K 2317/55; C07K 2317/569; C07K 2317/73; C07K 2317/92

(57) ABSTRACT

VHH-based NKp30 binders are provided having favorable characteristics. Moreover, pharmaceutical compositions including such a compound are produced and methods of medical treatment with such compounds and pharmaceutical compositions are developed. In a compound including a VHH antibody domain or fragment thereof, the VHH antibody domain or fragment thereof can have complementary determining regions CDR1, CDR2, and CDR3 of one of VHH1 to VHH16, or a humanized modification thereof, or a modification involving replacement, addition, and/or deletion of up to three amino acids in each complementary determining region.

### FIELD OF THE INVENTION

The present disclosure relates to VHH-based NKp30 binders with favorable characteristics. Moreover, the present disclosure relates to pharmaceutical compositions comprising such a compound and the use of such compounds and such pharmaceutical compositions in medical treatment methods.

### BACKGROUND OF THE INVENTION

Despite advances in clinical treatment in the past decades, cancer is still one of the leading causes of death in the developed world. In recent years, hope for considerable progress has been raised by novel approaches that harness the power of the immune system, in particular by activating immune cells in the human body and directing them against the tumor cells. One cell type that shows great potential in this regard are natural killer cells (NK cells).

NK cells play a pivotal role in early host defense against infections and tumors. NK cells are innate immune cells that were discovered in the 1970s based on their ability to exert antitumor cell cytotoxicity without prior sensitization of the host. In contrast to T-cells that recognize distinct antigens via their variable T-cell receptors, the discrimination between healthy and stressed cells and consequently the antitumor response of NK cells is based on a sophisticated interplay between a multitude of germline-encoded activating and inhibitory receptors (Gonzales-Rodriguez et al., 2019; Chiossone et al., 2018).

Natural killer cells are innate lymphocytes that recognize discontinuity and danger in multiple tissue compartments by integrating positive and negative signals. The negative signals are generally mediated by the interaction between self MHC-I on tissues and either Killer-Immunoglobulin-like Receptor (KIR) family members or Natural Killer Group 2A (NKG2A) (Carlsten et al., 2019; Vivier et al., 2008). Positive signals are transduced via the interaction of an array of NK activation receptors including the Natural Cytotoxicity Receptors (NCRs; NKp30, NKp46, NKp44), NKG2D and DNAM-1 as well as costimulatory molecules including 4-1BB and their ligands (Koch et al., 2017; Morgado et al., 2011). For the NCRs and NKG2D, many of the ligands are ‘danger signals’ that are upregulated on stressed and diseased tissues including viral infected cells and tumor cells. Another mechanism by which NK cells are activated is the bridging of the low affinity activating FcγRIIIa (CD16a) on NK cells with cells opsonized with IgG antibodies or bispecific antibodies. Unlike the NCRs and NKG2D, signaling through FcγRIIIa is often more robust in resting NK cells but is modulated by multiple variables including functionally distinct polymorphic variants of FcγRIIIa as well as competition for binding with circulating IgG. Ultimately, the balance of activation and inhibitory signal determines whether an NK cell will become activated. As such, NK cells have an endogenous capacity to differentiate between healthy and diseased tissues.

Ultimately, NK cell activation results in target cell lysis via degranulation i.e. release of cytotoxic substances such as perforin and granzymes as well as in the production of proinflammatory cytokines and chemokines.

NK cells have shown great potential for the treatment of cancer by different approaches.

Several early clinical trials employing the adoptive transfer of wild-type or genetically modified (e.g. CAR) NK cells either alone or in combination with antibodies as a therapeutic modality for cancer have shown encouraging early results for hematological malignancies (Gonzales-Rodriguez et al., 2019; Burger et al., 2019; Rezvani et al., 2019).

Although adoptive cell therapy with ex vivo activated NK cells represents a promising approach, the logistic complexity has also driven the development of NK directed antibody-based approaches to cancer immunotherapy. In this respect antibodies have been developed that block the interaction between inhibitory receptors on NK cells, e.g. NKG2A or KIR2DL1, KIR2DL2 or KIR2DL3, and their ligands enabling immune cell activation (André et al., 2018; Kohrt et al., 2014; Benson et al., 2015). Furthermore, the vast majority of NK cells express the low affinity Fcγ receptor CD16a. CD16a-ligation of an antibody bound to its target cell induces potent NK cell degranulation (Bryceson et al., 2005). This process, referred to as antibody-dependent cellular cytotoxicity (ADCC) is considered as one important mode of action of many therapeutic antibodies (Seidel et al., 2013).

The capability of an antibody to elicit ADCC however, is affected by antigen densities on target cells. Due to the low affinity interaction of antibodies with CD16a, low antigen densities typically result in minor degrees of opsonization and consequently in limited induction of ADCC (Koch et al., 2017). Moreover, CD16a polymorphisms in humans have been described that result in different levels of ADCC, depending on the patient's genotype. Finally, conventional therapeutic antibodies have to compete with serum immunoglobulins for CD16a binding, resulting in confined CD16a occupancy and restricted ADCC capacities (Ellwanger et al., 2019).

To overcome these inherent limitations of classical antibody therapies, bi- and multispecific NK cell engagers have been developed, in which one paratope binds to activating receptor CD16a with high affinities, while the other paratope is directed against a tumor-associated antigen (Koch et al., 2017; Rothe et al., 2015). In 2019, Vivier and co-workers described the efficient generation of trifunctional NK cell engagers (Gauthier et al., 2019). In their work, the authors employed two activating receptors of NK cells, NKp46 as well as CD16 (Fc-mediated) for effector cell engagement. In direct comparison with rituximab and Fc-engineered obinutuzumab in mouse in vivo studies, the developed NK cell engagers were more potent supporting the notion that this class of molecules might be promising therapeutic entities for tumor treatment.

Alternatively, natural or synthetic ligands of NK cell receptors can be combined with a tumor-targeting moiety in a bispecific format, thus constructing an effector cell engager. Such bispecific or trifunctional entities that form a bridge between an activating receptor on NK cells and a tumor associated antigen (TAA) on the tumor cell are referred to as NK cell engagers (Koch et al., 2017). Bispecific antibodies targeting a TAA (e.g. CD20) and NKp46, NKG2D and NKp30 (Peipp et al., 2015; Kellner et al., 2016) either via an antibody moiety or a recombinant form of the ectodomain of a ligand (e.g. ULBP2) (von Strandmann et al., 2006) have demonstrated potent target dependent cytotoxicity and cytokine release in vitro.

NKp30 is an activation receptor expressed on the majority of NK cells. Its cell bound ligand, B7-H6, is upregulated on tumor cells and absent on most normal cells. The other less well characterized ligand is HLA-B-associated transcript 3 (BAT3)/Bcl2-associated athanogene 6 (BAG6), which is expressed in the nucleus and can be transported to the plasma membrane or released in exosomes. Importantly, decreased NKp30 expression has been correlated with reduced survival in AML, and a lower number of NK cells expressing NKp30 were found in patients with gastric or breast cancer compared to healthy donors. Together, these data suggest that the NKp30 receptor axis may play an important role in tumor surveillance of different tumor entities. Therefore, potent strategies modulating the NKp30 axis may represent promising approaches to promote antitumor NK cell responses.

Despite the great potential of NK cell engagers, in practice their use has been limited because the available activating ligands for NK cells do not have a suitable profile of biophysical and/or functional characteristics (e.g. have an insufficient affinity for effective NK cell activation, cannot be produced economically in large amounts, do not have good stability when exposed to heat and solvents, and/or are not amenable to genetic manipulations for all desired uses, such as scaffolding, labeling, and altering specific amino acids).

Accordingly, there is a need in the art for improved ways to treat cancer. Moreover, there is a need in the art for improved ways to activate NK cells. Moreover, there is a need in the art for NK cell activating compounds, in particular for NK cell activating compounds that bind to NKp30, with improved characteristics, such as improved affinity, improved specificity, improved stability, improved manufacturability, improved amenability to genetic manipulations, improved potency and/or efficacy for the killing of tumor cells, increased effects in the release of proinflammatory cytokines, improved pharmacokinetics, reduced side effects, increased therapeutic window and/or increased patient safety. Moreover, there is a need in the art for NK cell activating compounds, in particular for NK cell activating compounds that bind to NKp30, that are not affected by the presence of natural B7-H6 ligand. Moreover, there is a need in the art to address the above-described needs by a “standardized” approach that can be widely used for activating NK cells (such as in combination with different targeting moieties) and/or that is inexpensive and allows for fast synthetic access.

The present disclosure overcomes the above-described problems and addresses the above-described needs.

### SUMMARY OF THE INVENTION

The present disclosure addresses the needs described above in the section “Background of the Invention” by the different aspects and embodiments described below.

The present invention is, in part, based on the surprising observation that compounds comprising a VHH antibody domain or fragment thereof as described in the present disclosure exhibit a combination of various advantageous effects. For example, advantageous effects can include (but are not limited to) a high affinity for NKp30, a high Kon rate for NKp30 binding, a low Koff rate for NKp30 binding, a high efficiency in activating NK cells, high efficiency in inducing cytokine release (interferon-γ, TNF-α), (in particular in the context of a molecule also including a targeting moiety), enhanced cytotoxicity (e.g. with regard to potency and/or efficacy) and improved manufacturability. If used in combination with an Fc region capable of FcγRIIIa binding, this cytotoxicity is further enhanced.

In an aspect, the present disclosure relates to a compound comprising a VHH antibody domain or a fragment thereof, wherein
(a) said VHH antibody domain or fragment thereof comprises the complementarity determining regions CDR1, CDR2 and CDR3 of one of VHH1 to VHH16 as shown in the Table of CDRs below;
(b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized; or
(c) said VHH antibody domain or fragment thereof comprises the complementarity determining regions CDR1, CDR2 and CDR3 as defined in (a) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3;

Table of CDRs:

| | CDR1 | CDR2 | CDR3 |
|---|---|---|---|
| VHH1 | ATSGFTLDDY | CFSPSD | AASFADGSS |
| | TIG | GTTY | WCYDYW |
| | (SEQ ID | (SEQ ID | (SEQ ID |
| | NO: 18) | NO: 19) | NO: 20) |
| VHH2 | AVSGQTWTNY | SIEWGGRGTY | AAQSSSRSPL |
| | HIG | A | ESNYDYW |
| | (SEQ ID | (SEQ ID | (SEQ ID |
| | NO: 21) | NO: 22) | NO: 23) |
| VHH3 | LVSGVTYHFY | CISYASGTTK | ATQGSGSCSD |
| | DIT | F | EMSAGSVLSW |
| | (SEQ ID | (SEQ ID | (SEQ ID |
| | NO: 24) | NO: 25) | NO: 26) |
| VHH4 | LVSGVTYHFY | CISYASGSTK | AMQGSGSCSD |
| | DIT | F | FMSADSVLSW |
| | (SEQ ID | (SEQ ID | (SEQ ID |
| | NO: 27) | NO: 28) | NO: 29) |
| VHH5 | LVSGVTYHFY | CISYASGSTK | ATQGSGSCSD |
| | DIT | F | FMSADSVLSW |
| | (SEQ ID | (SEQ ID | (SEQ ID |
| | NO: 30) | NO: 31) | NO: 32) |
| VHH6 | AASGLPFSKY | VVSWSGDKFY | AASGSYPVTD |
| | AIA | | RYFYNFW |
| | (SEQ ID | (SEQ ID | (SEQ ID |
| | NO: 33) | NO: 34) | NO: 35) |
| VHH7 | AASGFTFSSY | AAIFSAGGST | NKAPPFTTIQ |
| | DMG | IYA | PTCVMIDTQL |
| | (SEQ ID | (SEQ ID | GAW |
| | NO: 36) | NO: 37) | (SEQ ID |
| | | | NO: 38) |
| VHH8 | AASGFTFSSY | AAIFSADGST | NKAPPFTTIQ |
| | DMG | IYA | PMCVMIDTQL |
| | (SEQ ID | (SEQ ID | GAW |
| | NO: 39) | NO: 40) | (SEQ ID |
| | | | NO: 41) |
| VHH9 | AASGRGGASY | AAISWTGDTT | AAAHWTRVVA |
| | AMG | YN | KGAMADEKKY |
| | (SEQ ID | (SEQ ID | EYW |
| | NO: 42) | NO: 43) | (SEQ ID |
| | | | NO: 44) |
| VHH10 | AASGRTFSSN | AAIHWSGGSR | AAEISWQTTG |
| | EMG | AYA | WTNLGSW |
| | (SEQ ID | (SEQ ID | (SEQ ID |
| | NO: 45) | NO: 46) | NO: 47) |
| VHH11 | AASGRTESSD | AAIHWSGGST | AAEIRWLTTG |
| | EMG | SYA | WANLGSW |
| | (SEQ ID | (SEQ ID | (SEQ ID |
| | NO: 48) | NO: 49) | NO: 50) |
| VHH12 | AASGRTFSSD | AAIHWSGGSR | AAEIRWQTTG |
| | EMG | SYA | WANLGSW |
| | (SEQ ID | (SEQ ID | (SEQ ID |
| | NO: 51) | NO: 52) | NO: 53) |
| VHH13 | AASGRIFTSD | AAIHWSGGSR | AAEIRWQTAG |
| | EMG | SYA | WVNLGSW |
| | (SEQ ID | (SEQ ID | (SEQ ID |
| | NO: 54) | NO: 55) | NO: 56) |
| VHH14 | EASGVTFNFY | ADITSEGRTE | YTGG |
| | SMT | YA | SW |
| | (SEQ ID | (SEQ ID | (SEQ ID |
| | NO: 57) | NO: 58) | NO: 59) |
| VHH15 | AVSGSTFRLN | AGITADGGVT | NTAPAN |
| | VMG | NYA | WGSW |
| | (SEQ ID | (SEQ ID | (SEQ ID |
| | NO: 60) | NO: 61) | NO: 62) |
| VHH16 | TASGSIFSSL | AGITTDGSTD | NKSPYYDPTD |
| | TMAW | YA | YDSW |
| | (SEQ ID | (SEQ ID | (SEQ ID |
| | NO: 63) | NO: 64) | NO: 65) |

In another aspect, the present disclosure relates to a compound comprising a VHH antibody domain or a fragment thereof, wherein
(A) said VHH antibody domain comprises any one of the VHH sequences VHH1 to VHH16 shown in the Table of VHH Sequences below;
(B) said VHH antibody domain comprises a VHH sequence as defined in (A) with modification, wherein the modification is that said sequence is humanized;
(C) said VHH antibody domain comprises a VHH sequence as defined in (A) with modification, wherein the modification is the replacement, addition or deletion of up to 25 amino acids; or
(D) said VHH antibody domain comprises a VHH sequence that is at least 75% identical to a VHH sequence referred to in (A);

TABLE of
VHH Sequences:

| VHH Sequence | SEQ ID NO | Amino acid sequence |
|---|---|---|
| VHH1 | SEQ ID NO: 1 | DVQLQESGGGLVQPGGSLRLSCATSGFTLDDYT |
| | | IGWVRQAPGKEHEGVSCFSPSDGTTYYADSVKG |
| | | RFTFSRDNAKNTVYLQMNSLKPEDTAVYYCAAS |
| | | FADGSSWCYDYWGQGTQVTVSS |
| VHH2 | SEQ ID NO: 2 | DVQLQESGGGLVQAGGSLRLSCAVSGQTWTNYH |
| | | IGWFRQAPGKARESVASIEWGGRGTYATDSVKG |
| | | RFTISRDNAKNTVYLQMNSLKPEDTAVYYCAAQ |
| | | SSSRSPLESNYDYWGQGTQVTVSS |
| VHH3 | SEQ ID NO: 3 | DVQLQESGGGLVQPGESLKLSCLVSGVTYHFYD |
| | | ITWFRQAPGLEREGVSCISYASGTTKFADSVKG |
| | | RFTISRDNAKNAVYLEMNSLKPEDTAVYFCATQ |
| | | GSGSCSDEMSAGSVLSWGQGTQVTVSS |
| VHH4 | SEQ ID NO: 4 | DVQLQESGGGLVQPGESLRLSCLVSGVTYHFYD |
| | | ITWFRQAPGLEREGVSCISYASGSTKFADSVKG |
| | | RFTISRDNAKNAVYLQMNSLKPEDTAVYFCAMQ |
| | | GSGSCSDEMSADSVLSWGQGTQVTVSS |
| VHH5 | SEQ ID NO: 5 | DVQLQESGGGLVQPGESLRLSCLVSGVTYHFYD |
| | | ITWFRQAPGLEREGVSCISYASGSTKFADSVKG |
| | | RFTISRDNAKNAVYLQMNSLKPEDTAVYFCATQ |
| | | GSGSCSDEMSADSVLSWGQGTQVTVSS |
| VHH6 | SEQ ID NO: 6 | DVQLQESGGGLVQTGGSLRLSCAASGLPFSKYA |
| | | IAWFRQAPGKESEFLVVVSWSGDKFYQDNMKGR |
| | | CTISRDNAKNTAYLQINSLQPEDTAVYYCAASG |
| | | SYPVTDRYFYNFWGQGTQVTVSS |
| VHH7 | SEQ ID NO: 7 | DVQLQESGGGLAQPGGSLRLSCAASGFTESSYD |
| | | MGWYRQVPGKECELVAAIFSAGGSTIYADSVKG |
| | | RFTISRDNAKNTVFLQMNSLKPEDTAVYYCNKA |
| | | PPFTTIQPTCVMIDTQLGAWGQGTQVTVSS |
| VHH8 | SEQ ID NO: 8 | DVQLQESGGGLAQPGGSLRLSCAASGFTFSSYD |
| | | MGWYRQAPGKECELVAAIFSADGSTIYADSVKG |
| | | RFTISSDNAKNTVFLQMNSLKPEDTAVYYCNKA |
| | | PPFTTIQPMCVMIDTQLGAWGQGTQVTVSS |
| VHH9 | SEQ ID NO: 9 | DVQLQESGGGLVQAGGSLRLSCAASGRGGASYA |
| | | MGWFRQAPGKEREFVAAISWTGDTTYNVDSVKG |
| | | RFTISSDNAKNPVYLDMSSLKPEDTAVYYCAAA |
| | | HWTRVVAKGAMADEKKYEYWGQGTQVTVSS |
| VHH10 | SEQ ID NO: 10 | DVQLQESGGGSVQAGGSLRLSCAASGRTFSSNF |
| | | MGW FRQAPNKDREFVAAIHWSGGSRAYADSVKG |
| | | RFTISRDNTKDTVYLLVNSLKLEDTAVYYCAAE |
| | | ISWQTTGWTNLGSWGQGTQVTVSS |
| VHH11 | SEQ ID NO: 11 | DVQLQESGGGLVQAGGSLRLSCAASGRTFSSDF |
| | | MGWFRQAPGKEREFVAAIHWSGGSTSYADSVKG |
| | | RFTISRDNAKDTVYLLMNSLKLEDTAVYYCAAE |
| | | IRWLTTGWANLGSWGQGTQVTVSS |
| VHH12 | SEQ ID NO: 12 | DVQLQESGGGLVQAGGSLRLSCAASGRTFSSDF |
| | | MGWFRQAPGKEREFVAAIHWSGGSRSYADSVKG |
| | | RFTISRDNAKDTVYLLMNSLKLEDTAVYYCAAE |
| | | IRWQTTGWANLGSWGQGTQVTVSS |
| VHH13 | SEQ ID NO: 13 | DVQLQESGGGLAQPGGPLRLSCAASGRTFTSDF |
| | | MGWFRQAPGKEREFVAAIHWSGGSRSYADSVKG |
| | | RFTISRDNAKDTVYLLMNSLKLEDTAVYYCAAE |
| | | IRWQTAGWVNLGSWGQGTQVTVSS |
| VHH14 | SEQ ID NO: 14 | DVQLQESGGGLVQPGGSLRLSCEASGVTENFYS |
| | | MTWYRQAPGKERELVADITSEGRTEYAHSVKGR |
| | | FIISRDDFGTMVYLQMNSLKPEDTAVYYCYTGG |
| | | SWGQGTQVTVSS |
| VHH15 | SEQ ID NO: 15 | DVQLQESGGGLVQAGGSLRLSCAVSGSTFRLNV |
| | | MGWYRQAPGKLRDLVAGITADGGVTNYADSVKG |
| | | RFIISRDNAKNTVYLQMNSLKPEDTAIYYCNTA |
| | | PANWGSWGQGTQVTVSS |
| VHH16 | SEQ ID NO: 16 | DVQLQESGGGLVQPGGSLRLSCTASGSIFSSLT |
| | | MAWWRQAPGKQRELVAGITTDGSTDYADSVKGR |
| | | FTISRDNAKNTVYLQMDSLKPEDTAVYYCNKSP |
| | | YYDPTDYDSWGQGTQVTVSS |

In another aspect, the present disclosure relates to a compound comprising a VHH antibody domain or a fragment thereof, wherein
(A) said VHH antibody domain consists of any one of the VHH sequences VHH1 to VHH16 shown in the Table of VHH Sequences above;
(B) said VHH antibody domain consists of a VHH sequence as defined in (A) with modification, wherein the modification is that said sequence is humanized;
(C) said VHH antibody domain consists of a VHH sequence as defined in (A) with modification, wherein the modification is the replacement, addition or deletion of up to 25 amino acids; or
(D) said VHH antibody domain consists of a VHH sequence that is at least 75% identical to a VHH sequence referred to in (A).

In another aspect, the present disclosure relates to a pharmaceutical composition comprising the compound according to the present disclosure.

In another aspect, the present disclosure relates to a compound according to the present disclosure or a pharmaceutical composition according to the present disclosure for use as a medicament or for use in the treatment of a disease as defined below.

In another aspect, the present disclosure relates to a method for treating a disease in a patient in need thereof, comprising the step of administering to said patient a therapeutically effective amount of the compound according to the present disclosure or the pharmaceutical composition according to the present disclosure.

In another aspect, the present disclosure relates to the use of the compound according to the present disclosure or of the pharmaceutical composition according to the present disclosure for the manufacture of a medicament, preferably for the manufacture of a medicament for the treatment of a disease or disorder as defined below.

### BRIEF DESCRIPTION OF THE FIGURES

In the following, reference is made to the figures. All methods referred to in the figure descriptions below were carried out as described in detail in the examples.

FIG. 1 the generation of NKCEs (NK cell engagers) based on NKp30-specific VHH single domain antibodies by camelid immunization combined with yeast surface display (YSD). (A) Scheme depicting the generation of NKp30 targeting VHHs that are utilized for constructing bispecific NKCEs. After immunization of three camelids with recombinant human NKp30, YSD was employed for the isolation of antigen specific VHHs. Subsequently, VHHs were incorporated into bispecific SEEDbodies harboring the humanized Fab arm of Cetuximab for tumor targeting. For in depth characterization, Fc-mediated effector functions were silenced by the introduction of specific point mutations in the Fc region. (B) FACS selection for the isolation of NKp30-specific VHHs by implementing a two-dimensional staining strategy for full-length VHH display and NKp30 binding. Of note, in sorting round one all three sub-libraries based on each immunized specimen were sorted separately (only Llama-derived sub-library sort shown), followed by combining the sort one output for the subsequent sorting round two.

FIG. 2 shows results obtained in biolayer interferometry experiments to study competition of VHH SEEDbodies with B7-H6 or competition between different VHH SEEDbodies in binding to NKp30.

FIG. 3 shows the data of chromium release experiments, directing the conclusion that EGFR-positive tumor cells are potently killed with NKp30 VHH SEEDbodies. Standard 4 h chromium release assays were performed with high EGFR-expressing A431 cells (left graph) and lower EGFR-expression A549 cells (right graph) using MNC of healthy donors at an effector-to-target cell (E:T) ratio of 80:1 and increasing concentrations of B7-H6 competing (red colors) and non-competing VHH SEEDbodies (green/brown colors). A one-armed SEEDbody lacking the NKp30 VHH single domain antibody (oa_hu225-SEEDbody) and the monoclonal antibody cetuximab (black square) were used as controls and for comparison. Mean values±SEM of 3 independent experiments with triplicates are shown. Leading candidates for further analyses are depicted in bold.

FIG. 4 shows experiments to study tumor cell lysis and cytokine release by VHH SEEDbodies. B7-H6 competing VHH SEEDbodies (eff-) show improved lysis of tumor cells compared to non-competing VHH SEEDbodies (eff-), but all NKCES induce potent cytokine release from activated NK cells. (A) Standard 4 h chromium release assays were performed with A431 cells (left graph) and A549 cells (right graph) using isolated NK cells of healthy donors at an E:T ratio of 10:1 and increasing concentrations of B7-H6 competing (red colors) and non-competing VHH SEEDbodies (green/brown colors). A one-armed SEEDbody lacking the NKp30 VHH single domain antibody (oa_hu225SEEDbody) and the monoclonal antibody cetuximab (black square) were used as controls and for comparison. Mean values±SEM of 3 independent experiments with triplicates are shown. (B) Cytokine release triggered by selected VHH SEEDbodies (eff-) at saturating concentrations. NKCEs and cetuximab were compared to oa_hu225 SEEDbody with respect to promoting NK cell mediated IFN-γ and TNF-α release using cytokine HTRF kits for quantification. Purified NK cells were cocultured with A431 cells for 24 h at an ET ratio of 5:1 prior to analysis. Graphs show box and whisker plots as superimposition with dot plots of seven individual experiments, respectively. *** p, 0.001, ** p, 0.01, *p, 0.05. n.s., not significant compared to oa_hu225 SEEDbody.

FIG. 5 shows the data of experiments confirming that killing of EGFR-positive tumor cells with VHH SEEDbodies is not impaired by high concentrations of soluble B7-H6. Standard 4 h chromium release assays were performed with A431 (left graph) and A549 cells (right graph) using NK cells of healthy donors at an E:T ratio of 10:1 and saturating concentrations of the SEEDbodies in the presence (grey bars) or absence (black bars) of B7-H6 ECD. The one-armed SEEDbody lacking the NKp30 VHH single domain antibody (oa_hu225-SEEDbody), the monoclonal antibody cetuximab and B7-H6 ECD alone served as controls. Mean values±SEM of 4 independent experiments are shown. VHH1=B7-H6 competitor, VHH2=B7-H6 non-competitor, VHH4=partial competitor.

FIG. 6 shows the data of an experiment confirming that killing of EGFR-positive tumor cells by non-competing VHH SEEDbodies is improved by an effector functional Fc. Standard 4 h chromium release assays were performed with A431 cells (left graph) and A549 cells (right graph) using isolated NK cells of healthy donors at an ET ratio of 10:1 and increasing concentrations of B7-H6 competing VHH1 SEEDbody (orange colors) and non-competing VHH2 SEEDbody (green colors) harboring an effector functional (eff+; continuous line, filled symbols) or non-functional Fc (eff-; dotted line, open symbols). One-armed SEEDbodies lacking the NKp30 VHH single domain antibody, but carrying the effector functional (oa_hu225-SEEDbody eff+) or non-functional (oa_hu225-SEEDbody eff-) Fc as well as the monoclonal IgG1 antibody cetuximab (black square) were used as controls and for comparison. Mean values±SEM of 4 independent experiments with triplicates are shown.

SUMMARY OF SEQUENCES

| | Identifier/ | |
|---|---|---|
| SEQ ID NO | Description | Amino acid sequence |
| SEQ ID NO: 1 | Sequence of | DVQLQESGGGLVQPGGSLRLSCATSGFTLDDYTIGW |
| | VHH1 | VRQAPGKEHEGVSCFSPSDGTTYYADSVKGRFTFSR |
| | | DNAKNTVYLQMNSLKPEDTAVYYCAASFADGSSWCY |
| | | DYWGQGTQVTVSS |
| SEQ ID NO: 2 | Sequence of | DVQLQESGGGLVQAGGSLRLSCAVSGQTWTNYHIGW |
| | VHH2 | FRQAPGKARESVASIEWGGRGTYATDSVKGRFTISR |
| | | DNAKNTVYLQMNSLKPEDTAVYYCAAQSSSRSPLES |
| | | NYDYWGQGTQVTVSS |
| SEQ ID NO: 3 | Sequence of | DVQLQESGGGLVQPGESLKLSCLVSGVTYHFYDITW |
| | VHH3 | FRQAPGLEREGVSCISYASGTTKFADSVKGRFTISR |
| | | DNAKNAVYLEMNSLKPEDTAVYFCATQGSGSCSDEM |
| | | SAGSVLSWGQGTQVTVSS |
| SEQ ID NO: 4 | Sequence of | DVQLQESGGGLVQPGESLRLSCLVSGVTYHFYDITW |
| | VHH4 | FRQAPGLEREGVSCISYASGSTKFADSVKGRFTISR |
| | | DNAKNAVYLQMNSLKPEDTAVYFCAMQGSGSCSDEM |
| | | SADSVLSWGQGTQVTVSS |
| SEQ ID NO: 5 | Sequence of | DVQLQESGGGLVQPGESLRLSCLVSGVTYHFYDITW |
| | VHH5 | FRQAPGLEREGVSCISYASGSTKFADSVKGRFTISR |
| | | DNAKNAVYLQMNSLKPEDTAVYFCATQGSGSCSDEM |
| | | SADSVLSWGQGTQVTVSS |
| SEQ ID NO: 6 | Sequence of | DVQLQESGGGLVQTGGSLRLSCAASGLPFSKYAIAW |
| | VHH6 | FRQAPGKESEFLVVVSWSGDKFYQDNMKGRCTISRD |
| | | NAKNTAYLQINSLQPEDTAVYYCAASGSYPVTDRYF |
| | | YNFWGQGTQVTVSS |
| SEQ ID NO: 7 | Sequence of | DVQLQESGGGLAQPGGSLRLSCAASGFTFSSYDMGW |
| | VHH7 | YRQVPGKECELVAAIFSAGGSTIYADSVKGRFTISR |
| | | DNAKNTVFLQMNSLKPEDTAVYYCNKAPPFTTIQPT |
| | | CVMIDTQLGAWGQGTQVTVSS |
| SEQ ID NO: 8 | Sequence of | DVQLQESGGGLAQPGGSLRLSCAASGFTFSSYDMGW |
| | VHH8 | YRQAPGKECELVAAIFSADGSTIYADSVKGRFTISS |
| | | DNAKNTVFLQMNSLKPEDTAVYYCNKAPPFTTIQPM |
| | | CVMIDTQLGAWGQGTQVTVSS |
| SEQ ID NO: 9 | Sequence of | DVQLQESGGGLVQAGGSLRLSCAASGRGGASYAMGW |
| | VHH9 | FRQAPGKEREFVAAISWTGDTTYNVDSVKGRFTISS |
| | | DNAKNPVYLDMSSLKPEDTAVYYCAAAHWTRVVAKG |
| | | AMADEKKYEYWGQGTQVTVSS |
| SEQ ID NO: 10 | Sequence of | DVQLQESGGGSVQAGGSLRLSCAASGRTESSNEMGW |
| | VHH10 | FRQAPNKDREFVAAIHWSGGSRAYADSVKGRFTISR |
| | | DNTKDTVYLLVNSLKLEDTAVYYCAAEISWQTTGWT |
| | | NLGSWGQGTQVTVSS |
| SEQ ID NO: 11 | Sequence of | DVQLQESGGGLVQAGGSLRLSCAASGRTFSSDFMGW |
| | VHH11 | FRQAPGKEREFVAAIHWSGGSTSYADSVKGRFTISR |
| | | DNAKDTVYLLMNSLKLEDTAVYYCAAEIRWLTTGWA |
| | | NLGSWGQGTQVTVSS |
| SEQ ID NO: 12 | Sequence of | DVQLQESGGGLVQAGGSLRLSCAASGRTESSDEMGW |
| | VHH12 | FRQAPGKEREFVAAIHWSGGSRSYADSVKGRFTISR |
| | | DNAKDTVYLLMNSLKLEDTAVYYCAAEIRWQTTGWA |
| | | NLGSWGQGTQVTVSS |
| SEQ ID NO: 13 | Sequence of | DVQLQESGGGLAQPGGPLRLSCAASGRTFTSDEMGW |
| | VHH13 | FRQAPGKEREFVAAIHWSGGSRSYADSVKGRFTISR |
| | | DNAKDTVYLLMNSLKLEDTAVYYCAAEIRWQTAGWV |
| | | NLGSWGQGTQVTVSS |
| SEQ ID NO: 14 | Sequence of | DVQLQESGGGLVQPGGSLRLSCEASGVTENFYSMTW |
| | VHH14 | YRQAPGKERELVADITSEGRTEYAHSVKGRFIISRD |
| | | DFGTMVYLQMNSLKPEDTAVYYCYTGGSWGQGTQVT |
| | | VSS |
| SEQ ID NO: 15 | Sequence of | DVQLQESGGGLVQAGGSLRLSCAVSGSTFRLNVMGW |
| | VHH15 | YRQAPGKLRDLVAGITADGGVTNYADSVKGRFIISR |
| | | DNAKNTVYLQMNSLKPEDTAIYYCNTAPANWGSWGQ |
| | | GTQVTVSS |
| SEQ ID NO: 16 | Sequence of | DVQLQESGGGLVQPGGSLRLSCTASGSIFSSLTMAW |
| | VHH16 | WRQAPGKQRELVAGITTDGSTDYADSVKGRFTISRD |
| | | NAKNTVYLQMDSLKPEDTAVYYCNKSPYYDPTDYDS |
| | | WGQGTQVTVSS |
| SEQ ID NO: 17 | Human B7-H6* | MTWRAAASTCAALLILLWALTTEGDLKVEMMAGGTQ |
| | | ITPLNDNVTIFCNIFYSQPLNITSMGITWFWKSLTF |
| | | DKEVKVFEFFGDHQEAFRPGAIVSPWRLKSGDASLR |
| | | LPGIQLEEAGEYRCEVVVTPLKAQGTVQLEVVASPA |
| | | SRLLLDQVGMKENEDKYMCESSGFYPEAINITWEKQ |
| | | TQKFPHPIEISEDVITGPTIKNMDGTFNVTSCLKLN |
| | | SSQEDPGTVYQCVVRHASLHTPLRSNFTLTAARHSL |
| | | SETEKTDNESIHWWPISFIGVGLVLLIVLIPWKKIC |
| | | NKSSSAYTPLKCILKHWNSFDTQTLKKEHLIFFCTR |
| | | AWPSYQLQDGEAWPPEGSVNINTIQQLDVFCRQEGK |
| | | WSEVPYVQAFFALRDNPDLCQCCRIDPALLTVTSGK |
| | | SIDDNSTKSEKQTPREHSDAVPDAPILPVSPIWEPP |
| | | PATTSTTPVLSSQPPTLLLPLQ |
| SEQ ID NO: 18 | CDR1 of VHH1 | ATSGFTLDDYTIG |
| SEQ ID NO: 19 | CDR2 of VHH1 | CFSPSDGTTY |
| SEQ ID NO: 20 | CDR3 of VHH1 | AASFADGSSWCYDYW |
| SEQ ID NO: 21 | CDR1 of VHH2 | AVSGQTWINYHIG |
| SEQ ID NO: 22 | CDR2 of VHH2 | SIEWGGRGTYA |
| SEQ ID NO: 23 | CDR3 of VHH2 | AAQSSSRSPLESNYDYW |
| SEQ ID NO: 24 | CDR1 of VHH3 | LVSGVTYHFYDIT |
| SEQ ID NO: 25 | CDR2 of VHH3 | CISYASGTTKF |
| SEQ ID NO: 26 | CDR3 of VHH3 | ATQGSGSCSDEMSAGSVLSW |
| SEQ ID NO: 27 | CDR1 of VHH4 | LVSGVTYHFYDIT |
| SEQ ID NO: 28 | CDR2 of VHH4 | CISYASGSTKF |
| SEQ ID NO: 29 | CDR3 of VHH4 | AMQGSGSCSDEMSADSVLSW |
| SEQ ID NO: 30 | CDR1 of VHH5 | LVSGVTYHFYDIT |
| SEQ ID NO: 31 | CDR2 of VHH5 | CISYASGSTKF |
| SEQ ID NO: 32 | CDR3 of VHH5 | ATQGSGSCSDEMSADSVLSW |
| SEQ ID NO: 33 | CDR1 of VHH6 | AASGLPFSKYAIA |
| SEQ ID NO: 34 | CDR2 of VHH6 | VVSWSGDKFY |
| SEQ ID NO: 35 | CDR3 of VHH6 | AASGSYPVTDRYFYNFW |
| SEQ ID NO: 36 | CDR1 of VHH7 | AASGFTFSSYDMG |
| SEQ ID NO: 37 | CDR2 of VHH7 | AAIFSAGGSTIYA |
| SEQ ID NO: 38 | CDR3 of VHH7 | NKAPPFTTIQPTCVMIDTQLGAW |
| SEQ ID NO: 39 | CDR1 of VHH8 | AASGFTFSSYDMG |
| SEQ ID NO: 40 | CDR2 of VHH8 | AAIFSADGSTIYA |
| SEQ ID NO: 41 | CDR3 of VHH8 | NKAPPFTTIQPMCVMIDTQLGAW |
| SEQ ID NO: 42 | CDR1 of VHH9 | AASGRGGASYAMG |
| SEQ ID NO: 43 | CDR2 of VHH9 | AAISWTGDTTYN |
| SEQ ID NO: 44 | CDR3 of VHH9 | AAAHWTRVVAKGAMADEKKYEYW |
| SEQ ID NO: 45 | CDR1 of | AASGRTFSSNEMG |
| | VHH10 | |
| SEQ ID NO: 46 | CDR2 of | AAIHWSGGSRAYA |
| | VHH10 | |
| SEQ ID NO: 47 | CDR3 of | AAEISWQTTGWINLGSW |
| | VHH10 | |
| SEQ ID NO: 48 | CDR1 of | AASGRTFSSDFMG |
| | VHH11 | |
| SEQ ID NO: 49 | CDR2 of | AAIHWSGGSTSYA |
| | VHH11 | |
| SEQ ID NO: 50 | CDR3 of | AAEIRWLTTGWANLGSW |
| | VHH11 | |
| SEQ ID NO: 51 | CDR1 of | AASGRTFSSDFMG |
| | VHH12 | |
| SEQ ID NO: 52 | CDR2 of | AAIHWSGGSRSYA |
| | VHH12 | |
| SEQ ID NO: 53 | CDR3 of | AAEIRWQTTGWANLGSW |
| | VHH12 | |
| SEQ ID NO: 54 | CDR1 of | AASGRTFTSDEMG |
| | VHH13 | |
| SEQ ID NO: 55 | CDR2 of | AAIHWSGGSRSYA |
| | VHH13 | |
| SEQ ID NO: 56 | CDR3 of | AAEIRWQTAGWVNLGSW |
| | VHH13 | |
| SEQ ID NO: 57 | CDR1 of | EASGVTFNFYSMT |
| | VHH14 | |
| SEQ ID NO: 58 | CDR2 of | ADITSEGRTEYA |
| | VHH14 | |
| SEQ ID NO: 59 | CDR3 of | YTGGSW |
| | VHH14 | |
| SEQ ID NO: 60 | CDR1 of | AVSGSTFRLNVMG |
| | VHH15 | |
| SEQ ID NO: 61 | CDR2 of | AGITADGGVTNYA |
| | VHH15 | |
| SEQ ID NO: 62 | CDR3 of | NTAPANWGSW |
| | VHH15 | |
| SEQ ID NO: 63 | CDR1 of | TASGSIFSSLTMAW |
| | VHH16 | |
| SEQ ID NO: 64 | CDR2 of | AGITTDGSTDYA |
| | VHH16 | |
| SEQ ID NO: 65 | CDR3 of | NKSPYYDPTDYDSW |
| | VHH16 | |
| SEQ ID NO: 66 | human NKp30 | LWVSQPPEIRTLEGSSAFLPCSFNASQGRLAIGSVT |
| | extracellular | WERDEVVPGKEVRNGTPEFRGRLAPLASSRFLHDHQ |
| | domain | AELHIRDVRGHDASIYVCRVEVLGLGVGTGNGTRLV |
| | | VEKEHPQLG |
| SEQ ID NO: 67 | VHH1.1 | EVQLVESGGGVVQPGGSLRLSCATSGFTLDDYTIGW |
| | (humanized) | VRQAPGKEHEGVSCFSPSDGTTYYADSVKGRFTFSR |
| | | DNAKNTVYLQMNSLRAEDTAVYYCAASFADGSSWCY |
| | | DYWGQGTLVTVSS |
| SEQ ID NO: 68 | VHH1.2 | EVQLVESGGGVVQPGGSLRLSCATSGFTLDDYTIGW |
| | (humanized) | VRQAPGKEHEGVSCFSPSDGTTYYADSVKGRFTFSR |
| | | DNAKNTLYLQMNSLRAEDTAVYYCAASFADGSSWCY |
| | | DYWGQGTLVTVSS |
| SEQ ID NO: 69 | VHH1.3 | EVQLVESGGGVVQPGGSLRLSCAASGFTLDDYTIGW |
| | (humanized) | VRQAPGKEHEGVSCFSPSDGTTYYADSVKGRFTISR |
| | | DNAKNTLYLQMNSLRAEDTAVYYCAASFADGSSWCY |
| | | DYWGQGTLVTVSS |
| SEQ ID NO: 70 | VHH1.4 | EVQLVESGGGVVQPGGSLRLSCATSGFTLDDYTIGW |
| | (humanized) | VRQAPGKGLEGVSCFSPSDGTTYYADSVKGRFTESR |
| | | DNAKNTVYLQMNSLRAEDTAVYYCAASFADGSSWCY |
| | | DYWGQGTLVTVSS |
| SEQ ID NO: 71 | VHH1.5 | EVQLVESGGGVVQPGGSLRLSCAASGFTLDDYTIGW |
| | (humanized) | VRQAPGKGLEGVSCFSPSDGTTYYADSVKGRFTISR |
| | | DNAKNTVYLQMNSLRAEDTAVYYCAASFADGSSWCY |
| | | DYWGQGTLVTVSS |
| SEQ ID NO: 72 | VHH1.6 | EVQLVESGGGVVQPGGSLRLSCATSGFTLDDYTIGW |
| | (humanized) | VRQAPGKGLEGVSCFSPSDGTTYYADSVKGRFTFSR |
| | | DNAKNTLYLQMNSLRAEDTAVYYCAASFADGSSWCY |
| | | DYWGQGTLVTVSS |
| SEQ ID NO: 73 | VHH1.7 | EVQLVESGGGVVQPGGSLRLSCAASGFTLDDYTIGW |
| | (humanized) | VRQAPGKGLEGVSCFSPSDGTTYYADSVKGRFTFSR |
| | | DNAKNTLYLQMNSLRAEDTAVYYCAASFADGSSWCY |
| | | DYWGQGTLVTVSS |
| SEQ ID NO: 74 | VHH1.8 | EVQLVESGGGVVQPGGSLRLSCATSGFTLDDYTIGW |
| | (humanized) | VRQAPGKGLEGVSCFSPSDGTTYYADSVKGRFTISR |
| | | DNAKNTLYLQMNSLRAEDTAVYYCAASFADGSSWCY |
| | | DYWGQGTLVTVSS |
| SEQ ID NO: 75 | VHH1.9 | EVQLVESGGGVVQPGGSLRLSCAASGFTLDDYTIGW |
| | (humanized) | VRQAPGKGLEGVSCESPSDGTTYYADSVKGRFTISR |
| | | DNAKNTLYLQMNSLRAEDTAVYYCAASFADGSSWCY |
| | | DYWGQGTLVTVSS |
| SEQ ID NO: 76 | VHH1.10 | EVQLVESGGGVVQPGGSLRLSCAASGFTLDDYTIGW |
| | (humanized) | VRQAPGKGLEWVSCFSPSDGTTYYADSVKGRFTISR |
| | | DNAKNTLYLQMNSLRAEDTAVYYCAASFADGSSWCY |
| | | DYWGQGTLVTVSS |
| SEQ ID NO: 77 | VHH1.11 | EVQLVESGGGVVQPGGSLRLSCATSGFTLDDYTIGW |
| | (humanized) | VRQAPGKEHEGVSCFSPSDGTTYYADSVKGRFTISR |
| | | DNAKNTLYLQMNSLRAEDTAVYYCAASFADGSSWCY |
| | | DYWGQGTLVTVSS |
| SEQ ID NO: 78 | VHH1.12 | EVQLVESGGGVVQPGGSLRLSCAASGFTLDDYTIGW |
| | (humanized) | VRQAPGKEHEGVSCFSPSDGTTYYADSVKGRFTFSR |
| | | DNAKNTLYLQMNSLRAEDTAVYYCAASFADGSSWCY |
| | | DYWGQGTLVTVSS |
| SEQ ID NO: 79 | VHH2.1 | EVQLVESGGGVVQAGGSLRLSCAVSGQTWTNYHIGW |
| | (humanized) | FRQAPGKARESVASIEWGGRGTYATDSVKGRFTISR |
| | | DNAKNTVYLQMNSLRAEDTAVYYCAAQSSSRSPLES |
| | | NYDYWGQGTLVTVSS |
| SEQ ID NO: 80 | VHH2.2 | EVQLVESGGGVVQPGGSLRLSCAVSGQTWTNYHIGW |
| | (humanized) | FRQAPGKARESVASIEWGGRGTYATDSVKGRFTISR |
| | | DNAKNTVYLQMNSLRAEDTAVYYCAAQSSSRSPLES |
| | | NYDYWGQGTLVTVSS |
| SEQ ID NO: 81 | VHH2.3 | EVQLVESGGGVVQPGGSLRLSCAVSGQTWTNYHIGW |
| | (humanized) | FRQAPGKARESVASIEWGGRGTYATDSVKGRFTISR |
| | | DNAKNTLYLQMNSLRAEDTAVYYCAAQSSSRSPLES |
| | | NYDYWGQGTLVTVSS |
| SEQ ID NO: 82 | VHH2.4 | EVQLVESGGGVVQPGGSLRLSCAASGQTWTNYHIGW |
| | (humanized) | FRQAPGKARESVASIEWGGRGTYATDSVKGRFTISR |
| | | DNAKNTLYLQMNSLRAEDTAVYYCAAQSSSRSPLES |
| | | NYDYWGQGTLVTVSS |
| SEQ ID NO: 83 | VHH2.5 | EVQLVESGGGVVQPGGSLRLSCAVSGQTWTNYHIGW |
| | (humanized) | FRQAPGKGLESVASIEWGGRGTYATDSVKGRFTISR |
| | | DNAKNTVYLQMNSLRAEDTAVYYCAAQSSSRSPLES |
| | | NYDYWGQGTLVTVSS |
| SEQ ID NO: 84 | VHH2.6 | EVQLVESGGGVVQPGGSLRLSCAVSGQTWTNYHIGW |
| | (humanized) | VRQAPGKGLESVASIEWGGRGTYATDSVKGRFTISR |
| | | DNAKNTVYLQMNSLRAEDTAVYYCAAQSSSRSPLES |
| | | NYDYWGQGTLVTVSS |
| SEQ ID NO: 85 | VHH2.7 | EVQLVESGGGVVQPGGSLRLSCAVSGQTWINYHIGW |
| | (humanized) | FRQAPGKGLESVASIEWGGRGTYATDSVKGRFTISR |
| | | DNAKNTLYLQMNSLRAEDTAVYYCAAQSSSRSPLES |
| | | NYDYWGQGTLVTVSS |
| SEQ ID NO: 86 | VHH2.8 | EVQLVESGGGVVQPGGSLRLSCAVSGQTWTNYHIGW |
| | (humanized) | FRQAPGKGLEWVASIEWGGRGTYATDSVKGRFTISR |
| | | DNAKNTLYLQMNSLRAEDTAVYYCAAQSSSRSPLES |
| | | NYDYWGQGTLVTVSS |
| SEQ ID NO: 87 | VHH2.9 | EVQLVESGGGVVQPGGSLRLSCAVSGQTWTNYHIGW |
| | (humanized) | VRQAPGKGLESVASIEWGGRGTYATDSVKGRFTISR |
| | | DNAKNTLYLQMNSLRAEDTAVYYCAAQSSSRSPLES |
| | | NYDYWGQGTLVTVSS |
| SEQ ID NO: 88 | VHH2.10 | EVQLVESGGGVVQPGGSLRLSCAVSGQTWTNYHIGW |
| | (humanized) | VRQAPGKGLEWVASIEWGGRGTYATDSVKGRFTISR |
| | | DNAKNTLYLQMNSLRAEDTAVYYCAAQSSSRSPLES |
| | | NYDYWGQGTLVTVSS |
| SEQ ID NO: 89 | VHH2.11 | EVQLVESGGGVVQPGGSLRLSCAASGQTWINYHIGW |
| | (humanized) | FRQAPGKGLESVASIEWGGRGTYATDSVKGRFTISR |
| | | DNAKNTVYLQMNSLRAEDTAVYYCAAQSSSRSPLES |
| | | NYDYWGQGTLVTVSS |
| SEQ ID NO: 90 | VHH2.12 | EVQLVESGGGVVQPGGSLRLSCAASGQTWTNYHIGW |
| | (humanized) | FRQAPGKGLEWVASIEWGGRGTYATDSVKGRFTISR |
| | | DNAKNTVYLQMNSLRAEDTAVYYCAAQSSSRSPLES |
| | | NYDYWGQGTLVTVSS |
| SEQ ID NO: 91 | VHH2.13 | EVQLVESGGGVVQPGGSLRLSCAASGQTWTNYHIGW |
| | (humanized) | VRQAPGKGLESVASIEWGGRGTYATDSVKGRFTISR |
| | | DNAKNTVYLQMNSLRAEDTAVYYCAAQSSSRSPLES |
| | | NYDYWGQGTLVTVSS |
| SEQ ID NO: 92 | VHH2.14 | EVQLVESGGGVVQPGGSLRLSCAASGQTWINYHIGW |
| | (humanized) | VRQAPGKGLEWVASIEWGGRGTYATDSVKGRFTISR |
| | | DNAKNTVYLQMNSLRAEDTAVYYCAAQSSSRSPLES |
| | | NYDYWGQGTLVTVSS |
| SEQ ID NO: 93 | VHH2.15 | EVQLVESGGGVVQPGGSLRLSCAASGQTWTNYHIGW |
| | (humanized) | FRQAPGKGLESVASIEWGGRGTYATDSVKGRFTISR |
| | | DNAKNTLYLQMNSLRAEDTAVYYCAAQSSSRSPLES |
| | | NYDYWGQGTLVTVSS |
| SEQ ID NO: 94 | VHH2.16 | EVQLVESGGGVVQPGGSLRLSCAASGQTWTNYHIGW |
| | (humanized) | FRQAPGKGLEWVASIEWGGRGTYATDSVKGRFTISR |
| | | DNAKNTLYLQMNSLRAEDTAVYYCAAQSSSRSPLES |
| | | NYDYWGQGTLVTVSS |
| SEQ ID NO: 95 | VHH2.17 | EVQLVESGGGVVQPGGSLRLSCAASGQTWTNYHIGW |
| | (humanized) | VRQAPGKGLESVASIEWGGRGTYATDSVKGRFTISR |
| | | DNAKNTLYLQMNSLRAEDTAVYYCAAQSSSRSPLES |
| | | NYDYWGQGTLVTVSS |
| SEQ ID NO: 96 | VHH2.18 | EVQLVESGGGVVQPGGSLRLSCAASGQTWTNYHIGW |
| | (humanized) | VRQAPGKGLEWVASIEWGGRGTYATDSVKGRFTISR |
| | | DNAKNTLYLQMNSLRAEDTAVYYCAAQSSSRSPLES |
| | | NYDYWGQGTLVTVSS |

### DETAILED DESCRIPTION OF CERTAIN EMBODIMENTS

Although the present disclosure is described in detail above and below, it is to be understood that this disclosure is not limited to the particular methodologies, protocols and reagents described by the present disclosure, as these may vary. It is also to be understood that the terminology used herein is for the purpose of describing particular embodiments only, and is not intended to limit the scope of the present disclosure which will be limited only by the appended claims. Unless defined otherwise, all technical and scientific terms used herein have the same meanings as commonly understood by one of ordinary skill in the art.

In the following, certain elements of the present disclosure will be described in more detail, including the description of specific embodiments. However, the variously described examples and preferred embodiments should not be construed to limit the present disclosure to only the explicitly described embodiments. This description should be understood to support and encompass embodiments which combine the explicitly described embodiments with any number of the disclosed and/or preferred elements and in any manner. Furthermore, any permutations and combinations of all described elements in this application should be considered disclosed by the description of the present application except for where this leads to logical contradictions or the context indicates otherwise.

Unless defined otherwise herein, scientific and technical terms used in connection with the present disclosure shall have the meanings that are commonly understood by those of ordinary skill in the art. Generally, nomenclatures and techniques referred to in the present disclosure, e.g. nomenclatures and techniques of organic chemistry, chemical synthesis, biology, medicinal and pharmaceutical chemistry, medicine, pharmacology or toxicology, are those well-known and commonly used in the art. The methods and techniques of the present disclosure are generally performed according to conventional methods well-known in the art and as described in the references cited and discussed throughout the present disclosure unless otherwise indicated.

According to a first aspect, the present disclosure relates to a compound comprising a VHH antibody domain or a fragment thereof, wherein
(a) said VHH antibody domain or fragment thereof comprises the complementarity determining regions CDR1, CDR2 and CDR3 of one of VHH1 to VHH16 as shown in the Table of CDRs below;
(b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized; or
(c) said VHH antibody domain or fragment thereof comprises the complementarity determining regions CDR1, CDR2 and CDR3 as defined in (a) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3;

Table of CDRs:

| | CDR1 | CDR2 | CDR3 |
|---|---|---|---|
| VHH1 | ATSGFTLDDY | CFSPSDGTTY | AASFADGSSW |
| | TIG | | CYDYW |
| | (SEQ ID | (SEQ ID | (SEQ ID |
| | NO: 18) | NO: 19) | NO: 20) |
| VHH2 | AVSGQTWTNY | SIEWGGRGTY | AAQSSSRSPL |
| | HIG | A | ESNYDYW |
| | (SEQ ID | (SEQ ID | (SEQ ID |
| | NO: 21) | NO: 22) | NO: 23) |
| VHH3 | LVSGVTYHFY | CISYASGTTK | ATQGSGSCSD |
| | DIT | F | EMSAGSVLSW |
| | (SEQ ID | (SEQ ID | (SEQ ID |
| | NO: 24) | NO: 25) | NO: 26) |
| VHH4 | LVSGVTYHFY | CISYASGSTK | AMQGSGSCSD |
| | DIT | F | EMSADSVLSW |
| | (SEQ ID | (SEQ ID | (SEQ ID |
| | NO: 27) | NO: 28) | NO: 29) |
| VHH5 | LVSGVTYHFY | CISYASGSTK | ATQGSGSCSD |
| | DIT | F | EMSADSVLSW |
| | (SEQ ID | (SEQ ID | (SEQ ID |
| | NO: 30) | NO: 31) | NO: 32) |
| VHH6 | AASGLPFSKY | VVSWSGDKFY | AASGSYPVTD |
| | AIA | (SEQ ID | RY FYNFW |
| | (SEQ ID | NO: 34) | (SEQ ID |
| | NO: 33) | | NO: 35) |
| VHH7 | AASGFTFSSY | AAIFSAGGST | NKAPPFTTIQ |
| | DMG | IYA | PTCVMIDTQL |
| | (SEQ ID | (SEQ ID | GAW |
| | NO: 36) | NO: 37) | (SEQ ID |
| | | | NO: 38) |
| VHH8 | AASGFTFSSY | AAIFSADGST | NKAPPFTTIQ |
| | DMG | IYA | PMCVMIDTQL |
| | (SEQ ID | (SEQ ID | GAW |
| | NO: 39) | NO: 40) | (SEQ ID |
| | | | NO: 41) |
| VHH9 | AASGRGGASY | AAISWTGDTT | AAAHWTRVVA |
| | AMG | YN | KGAMADEKKY |
| | (SEQ ID | (SEQ ID | EYW |
| | NO: 42) | NO: 43) | (SEQ ID |
| | | | NO: 44) |
| VHH10 | AASGRTFSSN | AAIHWSGGSR | AAEISWQTTG |
| | EMG | AYA | WTNLGSW |
| | (SEQ ID | (SEQ ID | (SEQ ID |
| | NO: 45) | NO: 46) | NO: 47) |
| VHH11 | AASGRTESSD | AAIHWSGGST | AAEIRWLTTG |
| | EMG | SYA | WANLGSW |
| | (SEQ ID | (SEQ ID | (SEQ ID |
| | NO: 48) | NO: 49) | NO: 50) |
| VHH12 | AASGRTESSD | AAIHWSGGSR | AAEIRWQTTG |
| | EMG | SYA | WANLGSW |
| | (SEQ ID | (SEQ ID | (SEQ ID |
| | NO: 51) | NO: 52) | NO: 53) |
| VHH13 | AASGRTFTSD | AAIHWSGGSR | AAEIRWQTAG |
| | EMG | SYA | WVNLGSW |
| | (SEQ ID | (SEQ ID | (SEQ ID |
| | NO: 54) | NO: 55) | NO: 56) |
| VHH14 | EASGVTENFY | ADITSEGRTE | YTGG |
| | SMT | YA | SW |
| | (SEQ ID | (SEQ ID | (SEQ ID |
| | NO: 57) | NO: 58) | NO: 59) |
| VHH15 | AVSGSTFRLN | AGITADGGVT | NTAPAN |
| | VMG | NYA | WGSW |
| | (SEQ ID | (SEQ ID | (SEQ ID |
| | NO: 60) | NO: 61) | NO: 62) |
| VHH16 | TASGSIFSSL | AGITTDGSTD | NKSPYYDPTD |
| | TMAW | YA | YDSW |
| | (SEQ ID | (SEQ ID | (SEQ ID |
| | NO: 63) | NO: 64) | NO: 65) |

“Compound”, as used in the present disclosure, is not particularly limited and refers to a chemical entity of any chemical class, provided that it includes the protein domain as defined above. Thus, the compound can e.g. be an organic compound or a compound composed of an organic and an inorganic part, it can be a protein composed of a single amino acid chain, a protein composed of multiple amino acid chains that are either non-covalently or covalently associated, or a non-covalent complex including an inorganic component. The compound can consist of the amino acid sequence of the VHH antibody domain or fragment thereof alone or it can in addition include further amino acid(s) that may be covalently or non-covalently attached, or it can be associated with inorganic components. Preferably, the compound is a molecule.

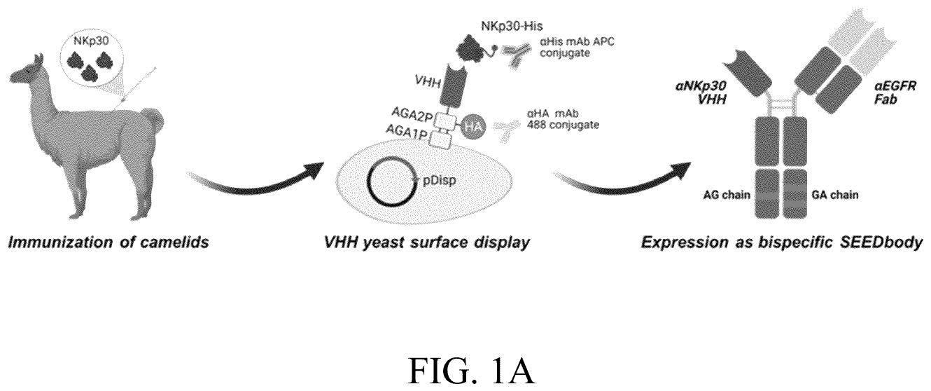

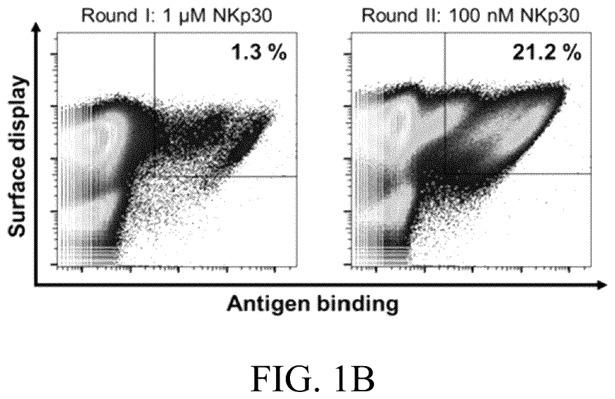

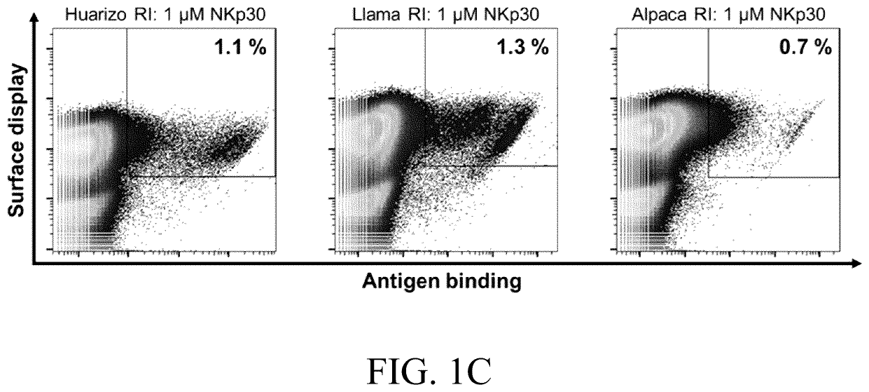

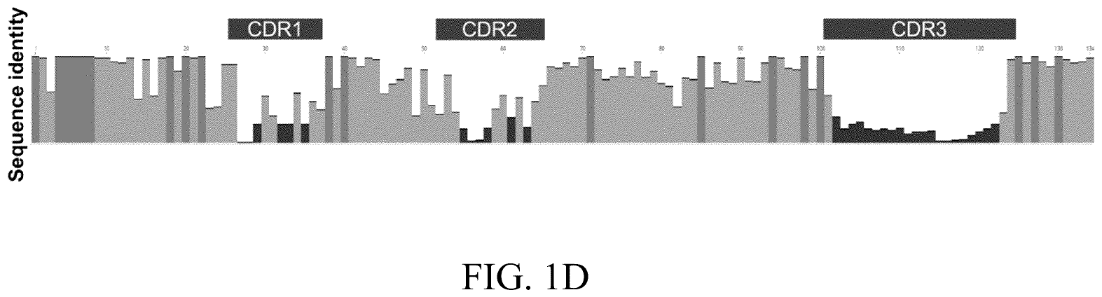

For example, the compound can be a bispecific molecule comprising a VHH antibody domain according to the present disclosure linked covalently to an IgG1 antibody lacking one of its “arms”. Or the compound may be an antibody with a VHH antibody domain according to the present disclosure and a targeting moiety prepared in the SEED format, resulting in a bispecific antibody with the structure shown in FIG. 1.

As the skilled person will understand, many other formats of the compound are possible, provided that the resulting compound does not interfere with the function of the VHH antibody domain or fragment thereof, i.e. binding to NKp30 and activation of NK cells.

The compound of the present disclosure can be prepared by standard methods of genetic engineering and recombinant protein technology known to the skilled person (see e.g. Green and Sambrook, “Molecular Cloning: A Laboratory Manual”, 2014; Coligan et al., “Current Protocols in Protein Science”, 1997). Exemplary methods are also described in the Examples section of the present disclosure.

In cases where the compound cannot be expressed in a single piece, individual parts can be prepared individually and later either covalently coupled, for example by a chemical reaction with appropriate reactive groups (e.g. linkage by maleimide chemistry) or by enzymatic linkage (e.g. transglutaminase-catalyzed linkage). For example, the VHH antibody domain or fragment thereof can be prepared by recombinant protein expression and subsequently linked to an antibody or antibody fragment, resulting in a bispecific antibody compound as described in the Examples section.

If the compound comprises components that are not biomolecules (such as a peptide mimetics or a small molecule), these components may be obtained e.g. by standard methods of synthetic organic chemistry.

An “antibody” is a polypeptide substantially encoded by an immunoglobulin gene or immunoglobulin genes, or antigen binding fragment thereof, which specifically binds and recognizes an analyte (antigen). Immunoglobulin genes include the kappa, lambda, alpha, gamma, delta, epsilon and mu constant region genes, as well as the myriad immunoglobulin variable region genes.

In primates such as humans, a heavy and the light chain variable domain of an antibody combine to specifically bind the antigen. Generally, a naturally occurring primate (e.g., human) or murine immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE. Primate antibodies can be class switched.

Certain IgG antibodies from members of the camel and dromedary (Camelus bactrianus and Calelus dromaderius) family including new world members such as llama species (Lama paccos, Lama glama and Lama vicugna) of mammals as found in nature lack light chains, and are thus structurally distinct from the typical four chain quaternary structure having two heavy and two light chains, for antibodies from other animals. See PCT/EP93/02214 (WO 94/04678 published 3 Mar. 1994). Such IgG subtypes of the llamas lack the light chains and the CH1 domain and are called heavy chain antibodies. These naturally occurring camelid antibodies consisting of only a heavy chain are functional and stable in the absence of light chain. The antigen-binding site of these heavy chain antibodies is formed only by a single domain, referred to as “VHH” (Könning et al., 2017) or, used synonymously herein, “VHH antibody domain”.

Each light and heavy chain of an antibody, contains constant domains and variable domains. References to “VH” or “VH” refer to the variable region of an immunoglobulin heavy chain, including that of an antibody fragment. References to “VL” or “VL” refer to the variable region of an immunoglobulin light chain, such as in a primate antibody. The variable domain of a heavy chain antibody is called VHH. The VHH is composed of only one polypeptide chain of 15 kDa and is considered the smallest known natural domain with full antigen-binding capacity.

Light and heavy chain variable domains contain a “framework” region interrupted by three hypervariable regions, also called “complementarity determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space. The CDRs are primarily responsible for antigen binding.

The CDRs are typically referred to as CDR1, CDR2, and CDR3 (from the N-terminus to C-terminus), and are also typically identified by the chain in which the particular CDR is located. Thus, a VH CDR3 is located in the variable domain of the heavy chain of the antibody in which it is found, whereas a VL CDR1 is the CDR1 from the variable domain of the light chain of the antibody in which it is found. Light chain CDRs are sometimes referred to as CDR L1, CDR L2, and CDR L3. Heavy chain CDRs are sometimes referred to as CDR H1, CDR H2, and CDR H3. VHH monoclonal antibodies have only a heavy chain, and thus include only one CDR1, CDR2 and CDR3. Generally, the CDR3 is primarily responsible for antigen specificity.

A VHH includes in an N- to C-direction, the following structural regions: N-FR1-CDR1-FR2-CDR2-FR3-CDR3-FR4-C, wherein FR denotes a framework region amino acid sequence and CDR denotes a complementary determining region amino acid sequence (see, e.g., Kabat et al., Sequences of Proteins of Immunological Interest, U.S. Department of Health and Human Services, 1991).

The extent of the framework region and CDRs have been defined (see, Kabat et al., Sequences of Proteins of Immunological Interest, U.S. Department of Health and Human Services, 1991). The CDRs of the heavy chain variable domain are located at residues 31-35 (CDR-H1), residues 50-65 (CDR-H2) and residues 95-102 (CDR-H3) according to the Kabat numbering system. In antibodies (such as primate antibodies) that include a light chain, such as a primate antibody, the CDRs of the light chain variable domain are located at residues 24-34 (CDR-L1), residues 50-56 (CDR-L2) and residues 89-97 (CDR-L3) according to the Kabat numbering system. The Kabat database is now maintained online. The location of camelid CDRs can also be determined (see, for example, Sircar et al., J. Immunol. 186: 6357-6367, 2011); a program to determine camelid antibody structure, the Rosetta Antibody program, is available on the internet.

A “monoclonal antibody” is an antibody produced by a single clone of B-lymphocytes or by a cell into which the heavy chain gene (and optionally a light chain gene, such as for a primate antibody) of a single antibody have been transfected. Monoclonal antibodies may be obtained using a variety of techniques known to those skilled in the art, including standard hybridoma technology (see e.g. Köhler and Milstein, Eur. J. Immunol. (1976), vol. 5, p. 511-519; Antibodies: A Laboratory Manual, 2nd edition (2014), editor Greenfield, Cold Spring Harbor Laboratory Press (USA); Immunobiology, 5th ed. (2001), editors Janeway et al., Garland Publishing (USA)) and e.g. expression from a eukaryotic host cell transfected with a DNA molecule coding for the homogeneous antibody or from a prokaryotic host cell transfected with a DNA molecule coding for the homogeneous antibody.

VHH antibody domains can be obtained by genetic engineering to yield a small protein having high affinity for a target, resulting in a low molecular weight antibody derived protein. See e.g. Sellmann et al., 2020; U.S. Pat. No. 5,759,808, issued Jun. 2, 1998; see also Dumoulin et al., (2003); Pleschberger et al., (2003); Cortez-Retamozo et al., (2002); and Lauwereys et al., (1998).

In some embodiments, the VHH molecules can be produced as recombinant monoclonal antibodies or antigen binding fragments in different expression platforms, avoiding the use of hybridomas and mice. VHH monoclonal antibodies can be humanized monoclonal antibodies. In some embodiments, monoclonal antibodies can be chimeric antibodies.

Without being bound by theory, a VHH monoclonal antibody has a molecular weight approximately one-tenth that of a human IgG molecule, and the protein has a physical diameter of only a few nanometers.

One consequence of the small size is the ability of the VHH monoclonal antibody to bind to antigenic sites that are functionally invisible to larger antibody proteins, such that VHH monoclonal antibodies are useful as reagents to detect antigens that are otherwise cryptic using classical immunological techniques, and thus are of use as therapeutic agents. Thus yet another consequence of small size is that a camelid VHH monoclonal antibody can inhibit as a result of binding to a specific site in a groove or narrow cleft of a target protein, and hence can serve in a capacity that more closely resembles the function of a classical low molecular weight drug than that of a classical antibody.

Without being bound by theory, low molecular weight and compact size further result in camelid VHH monoclonal antibodies being extremely thermostable, stable to extreme pH and to proteolytic digestion, and poorly antigenic. Further, these molecules can be fully expressed in prokaryotic cells such as E. coli and are expressed as fusion proteins with bacteriophage and are functional.

“Humanizing” an antibody/antibody sequence, as used herein, refers to the process where a non-human (such as camelid, llama or synthetic) antibody sequence is adapted to be more similar to a human antibody sequence by replacing one or more individual amino acids with the corresponding amino acids of a human antibody sequence. Typically, as human antibody sequence will be selected that is particularly close (i.e. has a high degree of sequence homology) to the non-human sequence. Such a human antibody sequence can be identified e.g. by a BLAST search. The corresponding amino acids can then be identified by a pairwise sequence alignment between the selected human antibody sequence and the non-human antibody sequence to be humanized. After humanization, the humanized antibody still binds to the same antigen as the original non-human antibody before humanization. Humanized immunoglobulins can be constructed by means of genetic engineering. A VHH antibody domain is easily humanized based on the human VH domain, which has a sequence that is highly homologous to the sequence of the VHH antibody domain.

VHHs can be used as modular building blocks for generating multivalent and/or multispecific antibody constructs, whereby “multivalent” means that the construct encompasses more than one single domain antibody and “multispecific” means that it encompasses single domain antibodies of more than one binding specificity.

At some occasions, the present disclosure states that a certain protein/amino acid sequence A is a “fragment” of another protein/amino acid sequence B. This means that, compared to protein/amino acid B, the protein/amino acid sequence A lacks one or more amino acids at the N-terminus and/or one or more amino acids at the C-terminus. Whether a protein/amino acid sequence lacks, compared to another protein/amino acid sequence, one or more amino acids at the N-terminus and/or one or more amino acids at the C-terminus can for example readily be determined upon forming a sequence alignment e.g. with the BLAST family of programs.

As the skilled person understands, when the present disclosure refers to an “VHH antibody domain or a fragment thereof”, said fragment is an antigen-binding fragment. Thus, said fragment binds to the same antigen as the “full-length” VHH antibody domain according to the present disclosure from which said fragment is derived (namely NKp30). Preferably, said fragment of said VHH antibody domain is a C-terminal fragment. This means that compared to the “complete” VHH antibody domain sequence said fragment lacks amino acids at the N-terminus.

When the present disclosure indicates that a VHH antibody domain or fragment thereof comprises the complementarity determining regions CDR1, CDR2 and CDR3 “of one of VHH1 to VHH16 as shown in the Table of CDRs”, this means that said VHH antibody domain comprises the combination of CDRs or either VHH1 or VHH2 or VHH3 etc., but not a mixture of CDRs selected from different of the listed VHHs. Thus, said VHH antibody domain or fragment thereof includes e.g. the combination of CDR1, CDR2 and CDR3 of VHH1 (SEQ ID NO: 18, 19 and 20), the combination of CDR1, CDR2 and CDR3 of VHH2 (SEQ ID NO: 21, 22, and 23), or the combination of CDR1, CDR2 and CDR3 of VHH3 (SEQ ID NO: 24, 25, and 26) etc., but not the combination of CDR1 and CDR2 of VHH1 and CDR3 of VHH2 (SEQ ID NO: 18, 19 and 23).

When the present disclosure defines that a VHH antibody domain or fragment thereof comprises the complementarity determining regions CDR1, CDR2 and CDR3 as defined in (a) with modification, wherein the modification is e.g. that the sequence of at least one of CDR1, CDR2 and CDR3 is humanized, the skilled person is aware that this humanization exists compared to the corresponding sequence in the Table of CDRs that provides the combinations of CDR sequences without modification.

If in the definition above the present disclosure indicates the existence of a modification which is “replacement, addition or deletion” of a certain number of amino acids (e.g. up to three), the skilled person understands that this is an individual replacement, addition or deletion. Thus, the replaced, added or deleted amino acids may be at neighboring positions or at independent, isolated positions within the amino acid sequence. Moreover, as above, the skilled person is aware that this definition indicates the replacement, addition or deletion compared to the unmodified sequence in the Table of CDRs.

In some embodiments, the modification in (b) is that the sequence of CDR1 and/or CDR2, but not the sequence of CDR3 is humanized.

In some embodiments, the modification in (b) is that the sequence of CDR1 is humanized, but not the sequence of CDR2 and CDR3.

In some embodiments, the modification in (b) is that the sequence of CDR2 is humanized, but not the sequence of CDR1 and CDR3.

In some embodiments, the modification in (b) is that the sequence of one, but not more than one of CDR1, CDR2 and CDR3 is humanized.

In some embodiments, said humanization of said CDR(s) is by replacing at least one amino acid in the sequence of said CDR by the corresponding amino acid of a human VH domain.

In some embodiments, said humanization of said CDR(s) is by replacing up to three amino acids in the sequence of said CDR by the corresponding amino acid of a human VH domain.

In some embodiments, said humanization of said CDR(s) is by replacing up to three amino acids in the sequence of CDR1 and/or CDR2 and up to one amino acid in the sequence of CDR3 by the corresponding amino acid of a human VH domain.

In some embodiments, said humanization of said CDR(s) is by replacing up to two amino acids in the sequence of said CDR by the corresponding amino acid of a human VH domain.

In some embodiments, said humanization of said CDR(s) is by replacing up to two amino acids in the sequence of CDR1 and/or CDR2 and up to one amino acid in the sequence of CDR3 by the corresponding amino acid of a human VH domain.

In some embodiments, said humanization of said CDR(s) is by replacing one amino acid in the sequence of said CDR by the corresponding amino acid of a human VH domain.

In some embodiments, the modification in (c) is
the replacement, addition or deletion of up to three amino acids in CDR1,
the replacement, addition or deletion of up to three amino acids in CDR2 and/or
the replacement, addition or deletion of up to one amino acid in CDR3.

In some embodiments, the modification in (c) is
the replacement, addition or deletion of up to two amino acids in CDR1,
the replacement, addition or deletion of up to two amino acids in CDR2 and/or
the replacement, addition or deletion of up to two amino acids in CDR3;

In some embodiments, the modification in (c) is
the replacement, addition or deletion of up to two amino acids in CDR1;
the replacement, addition or deletion of up to two amino acids in CDR2; and/or
the replacement, addition or deletion of up to one amino acid in CDR3.

In some embodiments, the modification in (c) is
the replacement, addition or deletion of up to two amino acids in CDR1 and/or
the replacement, addition or deletion of up to two amino acids in CDR2;
   wherein the sequence of CDR3 is unmodified

As a skilled person understands, the indication that the “sequence of CDR3 is unmodified” means that the sequence is unmodified compared to the sequence provided for CDR3 for the VHH at issue in the Table of CDRs.

In some embodiments, the modification in (c) is
the replacement, addition or deletion of up to two amino acids in CDR1,
   wherein the sequence of CDR2 and CDR3 is unmodified.

In some embodiments, the modification in (c) is
the replacement, addition or deletion of up to two amino acids in CDR2,
   wherein the sequence of CDR1 and CDR3 is unmodified.

In some embodiments, the modification in (c) is
the replacement, addition or deletion of up to one amino acid in CDR1;
the replacement, addition or deletion of up to one amino acid in CDR2; and/or
the replacement, addition or deletion of up to one amino acid in CDR3.

In some embodiments, the modification in (c) is
the replacement, addition or deletion of one amino acid in CDR1 and/or
the replacement, addition or deletion of one amino acid in CDR2;
   wherein the sequence of CDR3 is unmodified.

In some embodiments, the modification in (c) is
the replacement, addition or deletion of one amino acid in CDR1,
   wherein the sequence of CDR2 and CDR3 is unmodified.

In some embodiments, the modification in (c) is
the replacement, addition or deletion of one amino acid in CDR2,
   wherein the sequence of CDR1 and CDR3 is unmodified.

In some embodiments, the modification in (c) comprises only the replacement, but not the addition or deletion of amino acids. In some embodiments, said replacement is a conservative amino acid replacement.

As used herein, a “conservative amino acid replacement” refers to the replacement of an amino acid by another, biologically similar amino acid. Conservative replacements are not likely to change the shape or characteristics of a protein/amino acid sequence. Examples of conservative replacements include the replacement of one hydrophobic residue such as isoleucine, valine, leucine or methionine for another, or the substitution of one polar residue for another, such as the substitution of arginine for lysine, glutamic for aspartic acid, or glutamine for asparagine.

According to a second aspect, the present disclosure relates to a compound comprising a VHH antibody domain or a fragment thereof, wherein
(A) said VHH antibody domain comprises any one of the VHH sequences VHH1 to VHH16 shown in the Table of VHH Sequences below;
(B) said VHH antibody domain comprises a VHH sequence as defined in (A) with modification, wherein the modification is that said sequence is humanized;
(C) said VHH antibody domain comprises a VHH sequence as defined in (A) with modification, wherein the modification is the replacement, addition or deletion of up to 25 amino acids; or
(D) said VHH antibody domain comprises a VHH sequence that is at least 75% identical to a VHH sequence referred to in (A);

Table of VHH Sequences:

| VHH Sequence | SEQ ID NO | Amino acid sequence |
|---|---|---|
| VHH1 | SEQ ID NO: 1 | DVQLQESGGGLVQPGGSLRLSCATSGFTLDDYT |
| | | IGWVRQAPGKEHEGVSCFSPSDGTTYYADSVKG |
| | | RFTFSRDNAKNTVYLQMNSLKPEDTAVYYCAAS |
| | | FADGSSWCYDYWGQGTQVTVSS |
| VHH2 | SEQ ID NO: 2 | DVQLQESGGGLVQAGGSLRLSCAVSGQTWINYH |
| | | IGWFRQAPGKARESVASIEWGGRGTYATDSVKG |
| | | RFTISRDNAKNTVYLQMNSLKPEDTAVYYCAAQ |
| | | SSSRSPLESNYDYWGQGTQVTVSS |
| VHH3 | SEQ ID NO: 3 | DVQLQESGGGLVQPGESLKLSCLVSGVTYHFYD |
| | | ITWFRQAPGLEREGVSCISYASGTTKFADSVKG |
| | | RFTISRDNAKNAVYLEMNSLKPEDTAVYFCATQ |
| | | GSGSCSDEMSAGSVLSWGQGTQVTVSS |
| VHH4 | SEQ ID NO: 4 | DVQLQESGGGLVQPGESLRLSCLVSGVTYHFYD |
| | | ITWFRQAPGLEREGVSCISYASGSTKFADSVKG |
| | | RFTISRDNAKNAVYLQMNSLKPEDTAVYFCAMQ |
| | | GSGSCSDEMSADSVLSWGQGTQVTVSS |
| VHH5 | SEQ ID NO: 5 | DVQLQESGGGLVQPGESLRLSCLVSGVTYHFYD |
| | | ITWFRQAPGLEREGVSCISYASGSTKFADSVKG |
| | | RFTISRDNAKNAVYLQMNSLKPEDTAVYFCATQ |
| | | GSGSCSDEMSADSVLSWGQGTQVTVSS |
| VHH6 | SEQ ID NO: 6 | DVQLQESGGGLVQTGGSLRLSCAASGLPFSKYA |
| | | IAWFRQAPGKESEFLVVVSWSGDKFYQDNMKGR |
| | | CTISRDNAKNTAYLQINSLQPEDTAVYYCAASG |
| | | SYPVTDRYFYNFWGQGTQVTVSS |
| VHH7 | SEQ ID NO: 7 | DVQLQESGGGLAQPGGSLRLSCAASGFTFSSYD |
| | | MGWYRQVPGKECELVAAIFSAGGSTIYADSVKG |
| | | RFTISRDNAKNTVFLQMNSLKPEDTAVYYCNKA |
| | | PPFTTIQPTCVMIDTQLGAWGQGTQVTVSS |
| VHH8 | SEQ ID NO: 8 | DVQLQESGGGLAQPGGSLRLSCAASGFTFSSYD |
| | | MGWYRQAPGKECELVAAIFSADGSTIYADSVKG |
| | | RFTISSDNAKNTVFLQMNSLKPEDTAVYYCNKA |
| | | PPFTTIQPMCVMIDTQLGAWGQGTQVTVSS |
| VHH9 | SEQ ID NO: 9 | DVQLQESGGGLVQAGGSLRLSCAASGRGGASYA |
| | | MGWFRQAPGKEREFVAAISWTGDTTYNVDSVKG |
| | | RFTISSDNAKNPVYLDMSSLKPEDTAVYYCAAA |
| | | HWTRVVAKGAMADEKKYEYWGQGTQVTVSS |
| VHH10 | SEQ ID NO: 10 | DVQLQESGGGSVQAGGSLRLSCAASGRTFSSNF |
| | | MGWFRQAPNKDREFVAAIHWSGGSRAYADSVKG |
| | | RFTISRDNTKDTVYLLVNSLKLEDTAVYYCAAE |
| | | ISWQTTGWINLGSWGQGTQVTVSS |
| VHH11 | SEQ ID NO: 11 | DVQLQESGGGLVQAGGSLRLSCAASGRTFSSDF |
| | | MGWFRQAPGKEREFVAAIHWSGGSTSYADSVKG |
| | | RFTISRDNAKDTVYLLMNSLKLEDTAVYYCAAE |
| | | IRWLTTGWANLGSWGQGTQVTVSS |
| VHH12 | SEQ ID NO: 12 | DVQLQESGGGLVQAGGSLRLSCAASGRTESSDF |
| | | MGWFRQAPGKEREFVAAIHWSGGSRSYADSVKG |
| | | RFTISRDNAKDTVYLLMNSLKLEDTAVYYCAAE |
| | | IRWQTTGWANLGSWGQGTQVTVSS |
| VHH13 | SEQ ID NO: 13 | DVQLQESGGGLAQPGGPLRLSCAASGRTFTSDF |
| | | MGWFRQAPGKEREFVAAIHWSGGSRSYADSVKG |
| | | RFTISRDNAKDTVYLLMNSLKLEDTAVYYCAAE |
| | | IRWQTAGWVNLGSWGQGTQVTVSS |
| VHH14 | SEQ ID NO: 14 | DVQLQESGGGLVQPGGSLRLSCEASGVTENFYS |
| | | MTWYRQAPGKERELVADITSEGRTEYAHSVKGR |
| | | FIISRDDFGTMVYLQMNSLKPEDTAVYYCYTGG |
| | | SWGQGTQVTVSS |
| VHH15 | SEQ ID NO: 15 | DVQLQESGGGLVQAGGSLRLSCAVSGSTFRLNV |
| | | MGWYRQAPGKLRDLVAGITADGGVTNYADSVKG |
| | | RFIISRDNAKNTVYLQMNSLKPEDTAIYYCNTA |
| | | PANWGSWGQGTQVTVSS |
| VHH16 | SEQ ID NO: 16 | DVQLQESGGGLVQPGGSLRLSCTASGSIFSSLT |
| | | MAWWRQAPGKQRELVAGITTDGSTDYADSVKGR |
| | | FTISRDNAKNTVYLQMDSLKPEDTAVYYCNKSP |
| | | YYDPTDYDSWGQGTQVTVSS |

According to a third aspect, the present disclosure relates to a compound comprising a VHH antibody domain or a fragment thereof, wherein
(A) said VHH antibody domain consists of any one of the VHH sequences VHH1 to VHH16 shown in the Table of VHH Sequences;
(B) said VHH antibody domain consists of a VHH sequence as defined in (A) with modification, wherein the modification is that said sequence is humanized;
(C) said VHH antibody domain consists of a VHH sequence as defined in (A) with modification, wherein the modification is the replacement, addition or deletion of up to 25 amino acids; or
(D) said VHH antibody domain consists of a VHH sequence that is at least 75% identical to a VHH sequence referred to in (A).

To the second and third aspects the same explanations and definitions provided with respect to the first aspect above apply accordingly.

As the skilled person is aware, if the present disclosure indicates that a VHH antibody domain “comprises any one of the VHH sequences VHH1 to VHH16 shown in the Table of VHH Sequences”, this means that said VHH antibody domain comprises one and (not multiple or all) of the sequences listed in the Table of VHH Sequences.

Thus, if the present disclosure indicates that a VHH antibody domain or fragment thereof comprises/consists of a VHH sequence as defined in (A) with modification, wherein the modification is that said sequence is humanized, the skilled person is aware that this means that this humanization exists compared to the corresponding sequence in the Table of VHH Sequences that provides the sequences without modification.

If in the definition above the present disclosure indicates the existence of a modification which is “replacement, addition or deletion” of a certain number of amino acids (e.g. up to 25 amino acids), the skilled person understands that this is an individual replacement, addition or deletion. Thus, the replaced, added or deleted amino acids may be at neighboring positions or at independent, isolated positions within the amino acid sequence. Moreover, as above, the skilled person is aware that this definition indicates the replacement, addition or deletion compared to the unmodified sequence in the Table of VHH Sequences.

If the present disclosure states that a certain sequence A “is at least x % identical” to another sequence B, this is synonymous to the statement that sequence A “has x % identity” to sequence B. The statement reflects a relationship between the two polypeptide sequences A and B determined by comparing the sequences. In general, identity refers to an exact amino acid to amino acid correspondence of the two polypeptide sequences, respectively, over the length of the sequences being compared. For sequences where there is not an exact correspondence, a percentage to which the two sequences are identical may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity of two or more sequences are well known in the art. Thus, for instance, programs available in the Wisconsin Sequence Analysis Package, version 9.1 (Devereux J et al., 1984), for example the programs BESTFIT and GAP, may be used to determine the % identity between two polynucleotides and the % identity between two polypeptide sequences. BESTFIT uses the “local homology” algorithm of Smith and Waterman (1981) and finds the best single region of similarity between two sequences. Other programs for determining identity sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990). Preferably, % identity according to the present disclosure is determined according to the BLAST family of programs (Altschul S F et al, 1990, Altschul S F et al, 1997, accessible through the home page of the NCBI at www.ncbi.nlm.nih.gov).

In some embodiments, said fragment of said VHH antibody domain comprises at least 75% of the amino acids of the sequence of said VHH antibody domain. As the skilled person understands, this means that that fragment lacks up to a quarter of the total number of amino acids of said VHH antibody domain, wherein, compared to the “complete” VHH antibody domain sequence said amino acids are lacking either at the N-terminus or at the C-terminus.

In some embodiments, said fragment of said VHH antibody domain comprises at least 80% of the amino acids of the sequence of said VHH antibody domain.

In some embodiments, said fragment of said VHH antibody domain comprises at least 85% of the amino acids of the sequence of said VHH antibody domain.

In some embodiments, wherein said fragment of said VHH antibody domain comprises at least 90% of the amino acids of the sequence of said VHH antibody domain.

In some embodiments, said fragment of said VHH antibody domain comprises at least 95% of the amino acids of the sequence of said VHH antibody domain.

In some embodiments, said fragment of said VHH antibody domain comprises at least 98% of the amino acids of the sequence of said VHH antibody domain.

In some embodiments, said fragment of said VHH antibody domain comprises at least 99% of the amino acids of the sequence of said VHH antibody domain.

In some embodiments, said fragment of said VHH antibody domain comprises complementarity determining regions CDR1, CDR2 and CDR3.

In some embodiments, said fragment of said VHH antibody domain comprises at least the sequence from the N-terminus of CDR1 to the C-terminus of CDR3 of said VHH antibody domain.

In some embodiments, in (A) said fragment of said VHH antibody domain comprises all the complementarity determining regions (CDRs) of said VHH antibody domain.

In some embodiments, in (B) said humanization of said sequence is by replacing at least one amino acid of said sequence by the corresponding amino acid of a human VH (variable heavy) domain.

In some embodiments, in (B) said humanization of said sequence is by (individually) replacing up to 25 amino acids of said sequence by the corresponding amino acids of a human VH domain.

As the skilled person understands, if the present disclosure refers to the replacement of an amino acid of a certain sequence/domain A by the “corresponding” amino acid of a certain sequence/domain B, this designates that said amino acid of sequence/domain A is replaced by the amino acid in sequence/domain B that in an alignment of the two sequences aligns with said amino acid of sequence/domain A.

In some embodiments, in (B) said humanization of said sequence is by replacing up to 20 amino acids of said sequence by the corresponding amino acids of a human VH domain.

In some embodiments, in (B) said humanization of said sequence is by replacing up to 15 amino acids of said sequence by the corresponding amino acids of a human VH domain.

In some embodiments, in (B) said humanization of said sequence is by replacing up to 10 amino acids of said sequence by the corresponding amino acids of a human VH domain.

In some embodiments, in (B) said humanization of said sequence is by replacing up to 5 amino acids of said sequence by the corresponding amino acids of a human VH domain.

In some embodiments, in (B) said humanization of said sequence is by replacing up to 3 amino acids of said sequence by the corresponding amino acids of a human VH domain.

In some embodiments, in (B) said humanization of said sequence is by replacing up to 2 amino acids of said sequence by the corresponding amino acids of a human VH domain.

In some embodiments, in (B) said humanization of said sequence is by replacing one amino acid of said sequence by the corresponding amino acid of a human VH domain.

In some embodiments, in (B) said humanization is within the framework regions of said VHH antibody domain and/or within the CDRs of said VHH antibody domain.

In some embodiments, in (B) said humanization is within the framework regions of said VHH antibody domain, but not within the CDRs of said VHH antibody domain.

In some embodiments, in (B) said humanization is within the CDRs of said VHH antibody domain, but not within the framework regions of said VHH antibody domain.

In some embodiments, in (B) said humanization within the CDRs of said VHH antibody domain is within CDR1, CDR2 and/or CDR3.

In some embodiments, in (B) said humanization within the CDRs of said VHH antibody domain is within CDR1 and/or CDR2.

In some embodiments, in (B) said humanization within the CDRs of said VHH antibody domain is within CDR1.

In some embodiments, in (B) said humanization within the CDRs of said VHH antibody domain is within CDR2.

In some embodiments, in (B) said humanization within the CDRs of said VHH antibody domain is not within CDR3.

In some embodiments, in (C) the modification is the replacement, addition or deletion of up to 20 amino acids.

In some embodiments, in (C) the modification is the replacement, addition or deletion of up to 15 amino acids.

In some embodiments, in (C) the modification is the replacement, addition or deletion of up to 10 amino acids.

In some embodiments, in (C) the modification is the replacement, addition or deletion of up to 5 amino acids.

In some embodiments, in (C) the modification is the replacement, addition or deletion of up to 3 amino acids.

In some embodiments, in (C) the modification is the replacement, addition or deletion of up to 2 amino acids.

In some embodiments, in (C) the modification is the replacement, addition or deletion of one amino acid.

In some embodiments, the modification in (C) comprises only the replacement, but not the addition or deletion of amino acids.

In some embodiments, in (A) said VHH antibody domain or fragment thereof comprises the complementarity determining regions CDR1, CDR2 and CDR3 of one of VHH1 to VHH16 as shown in the Table of CDRs.

In some embodiments, in (B) to (D)
(a) said VHH antibody domain or fragment thereof comprises the complementarity determining regions CDR1, CDR2 and CDR3 of one of VHH1 to VHH16 as shown in the Table of CDRs;
(b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized;
(c) said VHH antibody domain or fragment thereof comprises the complementarity determining regions CDR1, CDR2 and CDR3 as defined in (a) with modification, wherein the modification is
   the replacement, addition or deletion of up to three amino acids in CDR1,
   the replacement, addition or deletion of up to three amino acids in CDR2 and/or
   the replacement, addition or deletion of up to three amino acids in CDR3.

In some embodiments, the modification in (b) is that the sequence of CDR1 and/or CDR2, but not the sequence of CDR3 is humanized.

In some embodiments, the modification in (b) is that the sequence of CDR1 is humanized, but not the sequence of CDR2 and CDR3.

In some embodiments, the modification in (b) is that the sequence of CDR2 is humanized, but not the sequence of CDR1 and CDR3.

In some embodiments, the modification in (b) is that the sequence of one, but not more than one of CDR1, CDR2 and CDR3 is humanized.

In some embodiments, said humanization of said CDR(s) is by replacing at least one amino acid in the sequence of said CDR by the corresponding amino acid of a human VH domain.

In some embodiments, said humanization of said CDR(s) is by replacing up to three amino acids in the sequence of said CDR by the corresponding amino acid of a human VH domain.

In some embodiments, said humanization of said CDR(s) is by replacing up to three amino acids in the sequence of CDR1 and/or CDR2 and up to one amino acid in the sequence of CDR3 by the corresponding amino acid of a human VH domain.

In some embodiments, said humanization of said CDR(s) is by replacing up to two amino acids in the sequence of said CDR by the corresponding amino acid of a human VH domain.

In some embodiments, said humanization of said CDR(s) is by replacing up to two amino acids in the sequence of CDR1 and/or CDR2 and up to one amino acid in the sequence of CDR3 by the corresponding amino acid of a human VH domain.

In some embodiments, said humanization of said CDR(s) is by replacing one amino acid in the sequence of said CDR by the corresponding amino acid of a human VH domain.

In some embodiments, the modification in (c) is
the replacement, addition or deletion of up to three amino acids in CDR1,
the replacement, addition or deletion of up to three amino acids in CDR2 and/or
the replacement, addition or deletion of up to two amino acids in CDR3.

In some embodiments, the modification in (c) is
the replacement, addition or deletion of up to three amino acids in CDR1,
the replacement, addition or deletion of up to three amino acids in CDR2 and/or
the replacement, addition or deletion of up to one amino acid in CDR3.

In some embodiments, the modification in (c) is
the replacement, addition or deletion of up to three amino acids in CDR1, and/or
the replacement, addition or deletion of up to three amino acids in CDR2,
   wherein the sequence of CDR3 is unmodified. As the skilled person is aware, “unmodified” mean unmodified compared to the sequence in the Table of CDRs.

In some embodiments, the modification in (c) is
the replacement, addition or deletion of up to two amino acids in CDR1,
the replacement, addition or deletion of up to two amino acids in CDR2 and/or
the replacement, addition or deletion of up to two amino acids in CDR3;

In some embodiments, the modification in (c) is
the replacement, addition or deletion of up to two amino acids in CDR1;
the replacement, addition or deletion of up to two amino acids in CDR2; and/or
the replacement, addition or deletion of up to one amino acid in CDR3.

In some embodiments, the modification in (c) is
the replacement, addition or deletion of up to two amino acids in CDR1 and/or
the replacement, addition or deletion of up to two amino acids in CDR2;
   wherein the sequence of CDR3 is unmodified.

In some embodiments, the modification in (c) is
the replacement, addition or deletion of up to two amino acids in CDR1,
   wherein the sequence of CDR2 and CDR3 is unmodified.

In some embodiments, the modification in (c) is
the replacement, addition or deletion of up to two amino acids in CDR2,
   wherein the sequence of CDR1 and CDR3 is unmodified.

In some embodiments, the modification in (c) is
the replacement, addition or deletion of up to one amino acid in CDR1;
the replacement, addition or deletion of up to one amino acid in CDR2; and/or
the replacement, addition or deletion of up to one amino acid in CDR3.

In some embodiments, the modification in (c) is
the replacement, addition or deletion of one amino acid in CDR1 and/or
the replacement, addition or deletion of one amino acid in CDR2;
   wherein the sequence of CDR3 is unmodified.

In some embodiments, the modification in (c) is
the replacement, addition or deletion of one amino acid in CDR1,
   wherein the sequence of CDR2 and CDR3 is unmodified.

In some embodiments, the modification in (c) is
the replacement, addition or deletion of one amino acid in CDR2,
   wherein the sequence of CDR1 and CDR3 is unmodified.

In some embodiments, the modification in (c) comprises only the replacement, but not the addition or deletion of amino acids.

In some embodiments, said compound comprises a VHH antibody domain (not only a fragment thereof).

In some embodiments, said compound consists of a fragment of a VHH antibody domain (not a full-length VHH antibody domain).

In some embodiments, said fragment consists of at least 100 amino acids.

In some embodiments, said fragment consists of at least 105 amino acids.

In some embodiments, said fragment consists of at least 110 amino acids.

In some embodiments, said fragment consists of at least 115 amino acids.

In some embodiments, said compound is capable of specifically binding to NKp30.

In some embodiments, the specific term that a certain compound, domain or fragment “is capable of specifically binding to NKp30” means that said compound, domain or fragment is capable of binding to NKp30 with a affinity that is at least equal to the affinity with which human B7-H6 (SEQ ID NO: 17) binds to NKp30. Such binding can be determined by in vitro binding experiments as described in Example 1 below (by biolayer interferometry).

In some embodiments, said VHH antibody domain or fragment thereof is capable of specifically binding to NKp30.

In some embodiments, said VHH antibody domain or fragment thereof binds to recombinant human NKp30 with a KD of 1×10⁻⁶ M or stronger.

In some embodiments, said VHH antibody domain or fragment thereof binds to recombinant human NKp30 with a KD of 1×10⁻⁷ M or stronger.

In some embodiments, said VHH antibody domain or fragment thereof binds to recombinant human NKp30 with a KD of 1×10⁻⁸ M or stronger.

In some embodiments, said VHH antibody domain or fragment thereof binds to recombinant human NKp30 with a KD of 1×10⁻⁹ M or stronger.

In some embodiments, said VHH antibody domain or fragment thereof binds to recombinant human NKp30 with a kₒₙ of 1×10⁴ (1/Ms) or higher.

In some embodiments, said VHH antibody domain or fragment thereof binds to recombinant human NKp30 with a kₒₙ of 1×10⁵ (1/Ms) or higher.

In some embodiments, said VHH antibody domain or fragment thereof binds to recombinant human NKp30 with a k_{off} of 5×10⁻³ (1/s) or lower.

In some embodiments, said VHH antibody domain or fragment thereof binds to recombinant human NKp30 with a k_{off} of 1×10⁻³ (1/s) or lower.

In some embodiments, said VHH antibody domain or fragment thereof binds to recombinant human NKp30 with a k_{off} of 1×10-+(1/s) or lower.

In some embodiments, said VHH antibody domain or fragment thereof binds to recombinant human NKp30 with a k_{off} of 1×10⁻⁵ (1/s) or lower.

In some embodiments, said VHH antibody domain or fragment thereof binds to recombinant human NKp30 with a k_{off} of 1×10⁻⁶ (1/s) or lower.

In some embodiments, said VHH antibody domain or fragment thereof binds to recombinant human NKp30 with a k_{off} of 1×10⁻⁷ (1/s) or lower.

In some embodiments, in (b) and (c) the VHH antibody domain or fragment thereof binds to recombinant human NKp30 with an affinity (KD value) that is by not more than a factor of 5 weaker than the binding of the corresponding VHH antibody domain without modification.

In some embodiments, in (b) and (c) the VHH antibody domain or fragment thereof binds to recombinant human NKp30 with an affinity (KD value) that is by not more than a factor of 2 weaker than the binding of the corresponding VHH antibody domain without modification.

In some embodiments, in (b) and (c) the VHH antibody domain or fragment thereof binds to recombinant human NKp30 with an affinity (KD value) that is by not more than a factor of 1.5 weaker than the binding of the corresponding VHH antibody domain without modification.

In some embodiments, in (b) and (c) the VHH antibody domain or fragment thereof binds to recombinant human NKp30 with an affinity (KD value) that is by not more than a factor of 5 stronger than the binding of the corresponding VHH antibody domain without modification.

In some embodiments, in (b) and (c) the VHH antibody domain or fragment thereof binds to recombinant human NKp30 with an affinity (KD value) that is by not more than a factor of 2 stronger than the binding of the corresponding VHH antibody domain without modification.

In some embodiments, in (b) and (c) the VHH antibody domain or fragment thereof binds to recombinant human NKp30 with an affinity (KD value) that is by not more than a factor of 1.5 stronger than the binding of the corresponding VHH antibody domain without modification.

In some embodiments, in (B) and (C) the VHH antibody domain binds to recombinant human NKp30 with an affinity (KD value) that is by not more than a factor of 5 weaker than the binding of the corresponding VHH antibody domain without modification.

In some embodiments, in (B) and (C) the VHH antibody domain binds to recombinant human NKp30 with an affinity (KD value) that is by not more than a factor of 2 weaker than the binding of the corresponding VHH antibody domain without modification.

In some embodiments, in (B) and (C) the VHH antibody domain binds to recombinant human NKp30 with an affinity (KD value) that is by not more than a factor of 1.5 weaker than the binding of the corresponding VHH antibody domain without modification.

In some embodiments, in (B) and (C) the VHH antibody domain binds to recombinant human NKp30 with an affinity (KD value) that is by not more than a factor of 5 stronger than the binding of the corresponding VHH antibody domain without modification.

In some embodiments, in (B) and (C) the VHH antibody domain binds to recombinant human NKp30 with an affinity (KD value) that is by not more than a factor of 2 stronger than the binding of the corresponding VHH antibody domain without modification.

In some embodiments, in (B) and (C) the VHH antibody domain binds to recombinant human NKp30 with an affinity (KD value) that is by not more than a factor of 1.5 stronger than the binding of the corresponding VHH antibody domain without modification.

In some embodiments, in (D) the affinity (KD value) of the binding of the VHH antibody domain to human NKp30 is by not more than a factor of 5 weaker than the affinity (KD value) of the binding to human NKp30 of a VHH antibody domain consisting of the sequence from the Table of VHH Sequences that has the highest degree of sequence identity with the sequence of said VHH antibody domain of (D). The degree of sequence identity can be determined by sequence alignment.

In some embodiments, in (D) the affinity (KD value) of the binding of the VHH antibody domain to human NKp30 is by not more than a factor of 2 weaker than the affinity (KD value) of the binding to human NKp30 of a VHH antibody domain consisting of the sequence from the Table of VHH Sequences that has the highest degree of sequence identity with the sequence of said VHH antibody domain of (D).

In some embodiments, in (D) the affinity (KD value) of the binding of the VHH antibody domain to human NKp30 is by not more than a factor of 1.5 weaker than the affinity (KD value) of the binding to human NKp30 of a VHH antibody domain consisting of the sequence from the Table of VHH Sequences that has the highest degree of sequence identity with the sequence of said VHH antibody domain of (D).

In some embodiments, in (D) the affinity (KD value) of the binding of the VHH antibody domain to human NKp30 is by not more than a factor of 5 stronger than the affinity (KD value) of the binding to human NKp30 of a VHH antibody domain consisting of the sequence from the Table of VHH Sequences that has the highest degree of sequence identity with the sequence of said VHH antibody domain of (D).

In some embodiments, in (D) the affinity (KD value) of the binding of the VHH antibody domain to human NKp30 is by not more than a factor of 2 stronger than the affinity (KD value) of the binding to human NKp30 of a VHH antibody domain consisting of the sequence from the Table of VHH Sequences that has the highest degree of sequence identity with the sequence of said VHH antibody domain of (D).

In some embodiments, in (D) the affinity (KD value) of the binding of the VHH antibody domain to human NKp30 is by not more than a factor of 1.5 stronger than the affinity (KD value) of the binding to human NKp30 of a VHH antibody domain consisting of the sequence from the Table of VHH Sequences that has the highest degree of sequence identity with the sequence of said VHH antibody domain of (D).

In some embodiments, said KD value/said kₒₙ rate/said k_{off} rate is measured by kinetic measurements by biolayer interferometry at 25° C. and 1000 rpm in KB Buffer (PBS+0.1% Tween-20+1% BSA). In some embodiments, binding (i.e. binding/specific binding as such) is determined by this approach.

In some embodiments, said VHH antibody domain or fragment thereof competes with human B7-H6 for binding to human NKp30. If a VHH antibody domain or fragment thereof competes with human B7-H6 can be determined as described in the Examples section.

In some embodiments, said VHH antibody domain or fragment thereof does not compete with human B7-H6 for binding to human NKp30.

In some embodiments, said VHH antibody domain or fragment thereof partially competes with human B7-H6 for binding to human NKp30.

In some embodiments, said VHH antibody domain of (A) comprises any one of the VHH sequences VHH1, VHH2, VHH3, VHH4, VHH5, VHH8, VHH9, VHH10, VHH11, VHH12, VHH13, VHH14, VHH15 or VHH16 shown in the Table of VHH Sequences. (As can be seen e.g. from Example 3, Table 1, for compounds with such a VHH antibody domain a KD in the range of E-09 or stronger was observed for binding to NKp30.)

In some embodiments, said VHH antibody domain of (A) comprises any one of the VHH sequences VHH1, VHH3, VHH5, VHH8, VHH10, VHH12, VHH13, VHH14, VHH15 or VHH16 shown in the Table of VHH Sequences. (As can be seen e.g. from Example 3, Table 1, for compounds with such a VHH antibody domain a KD in the range of E-10 or stronger was observed for binding to NKp30.)

In some embodiments, said VHH antibody domain of (A) comprises the VHH sequence VHH15 shown in the Table of VHH Sequences. (As can be seen e.g. from Example 3, Table 1, for compounds with such a VHH antibody domain a KD in the range of E-12 or stronger and a k_{off} in the range of E-07 or lower was observed for binding to NKp30.)

In some embodiments, said VHH antibody domain of (A) comprises any one of the VHH sequences VHH1, VHH2, VHH3, VHH5, VHH6, VHH8, VHH9, VHH10, VHH11, VHH12, VHH13, VHH14, VHH15 or VHH16 shown in the Table of VHH Sequences. (As can be seen e.g. from Example 3, Table 1, for compounds with such a VHH antibody domain a kₒₙ in the range of E-05 was observed for binding to NKp30.)

In some embodiments, said VHH antibody domain of (A) comprises any one of the VHH sequences VHH1, VHH2, VHH3, VHH4, VHH5, VHH8, VHH10, VHH11, VHH12, VHH13, VHH14, VHH15 or VHH16 shown in the Table of VHH Sequences. (As can be seen e.g. from Example 3, Table 1, for compounds with such a VHH antibody domain a k_{off} in the range of E-04 or lower was observed for binding to NKp30.)

In some embodiments, said VHH antibody domain of (A) comprises any one of the VHH sequences VHH10, VHH15 or VHH16 shown in the Table of VHH Sequences. (As can be seen e.g. from Example 3, Table 1, for compounds with such a VHH antibody domain a k_{off} in the range of E-05 or lower was observed for binding to NKp30.)

In some embodiments, said VHH antibody domain of (A) comprises any one of the VHH sequences VHH1, VHH3, VHH5, VHH12, VHH13, VHH14, VHH15 or VHH16 shown in the Table of VHH Sequences. (As can be seen e.g. from Example 3, Table 1, for compounds with such a VHH antibody domain competition with B7-H6 is observed. As shown in Example 4, such VHH antibody domains result in compounds that mediate lysis of targeted cells with high efficiency.)

In some embodiments, said VHH antibody domain of (A) comprises any one of the VHH sequences VHH1, VHH5 or VHH16 shown in the Table of VHH Sequences.

In some embodiments, said VHH antibody domain of (A) comprises any one of the VHH sequences VHH2, VHH6, VHH7, VHH8, VHH9, VHH10 or VHH11 shown in the Table of VHH Sequences. (As can be seen e.g. from Example 3, Table 1, for compounds with such a VHH antibody domain no competition with B7-H6 is observed. As shown in Example 4, such VHH antibody domains result in compounds that mediate lysis of targeted cells, but with a lower efficiency.)

In some embodiments, said VHH antibody domain of (A) comprises any one of the VHH sequences VHH2, VHH4 or VHH8 shown in the Table of VHH Sequences.

In some embodiments, said VHH antibody domain of (A) comprises any one of the VHH sequences VHH2 or VHH8 shown in the Table of VHH Sequences.

In some embodiments, said VHH antibody domain of (A) comprises the VHH sequence VHH4 shown in the Table of VHH Sequences. (As can be seen e.g. from Example 3, Table 1, for compounds with such a VHH antibody domain partial competition with B7-H6 is observed.)

In some embodiments, said VHH antibody domain of (A) comprises any one of the VHH sequences VHH12 or VHH15 shown in the Table of VHH Sequences. (As can be seen e.g. from Example 3, for compounds with such a VHH antibody domain no competition with the partial competitor VHH4 is observed.)

In some embodiments, said VHH antibody domain of (A) comprises any one of the VHH sequences VHH8, VHH9, VHH10 or VHH11 shown in the Table of VHH Sequences. (As can be seen e.g. from Example 3, compounds with such a VHH antibody domain belong to a specific epitope bin with common characteristics.)

In some embodiments, said VHH antibody domain of (A) comprises any one of the VHH sequences VHH2 or VHH6 shown in the Table of VHH Sequences. (As can be seen e.g. from Example 3, compounds with such a VHH antibody domain belong to a specific epitope bin with common characteristics.)

In some embodiments, said VHH antibody domain of (A) comprises any one of the VHH sequences VHH2, VHH6 or VHH7 shown in the Table of VHH Sequences. (As can be seen e.g. from Example 3, compounds with such a VHH antibody domain are unique with respect to their epitope targeting.)

In some embodiments, in (a) said VHH antibody domain or fragment thereof comprises the complementarity determining regions CDR1, CDR2 and CDR3 of one of any one of the VHH sequences VHH1, VHH2, VHH3, VHH4, VHH5, VHH8, VHH9, VHH10, VHH11, VHH12, VHH13, VHH14, VHH15 or VHH16 shown in the Table of CDRs. (As can be seen e.g. from Example 3, Table 1, for compounds with such a VHH antibody domain a KD in the range of E-09 or stronger was observed for binding to NKp30.)

In some embodiments, in (a) said VHH antibody domain or fragment thereof comprises the complementarity determining regions CDR1, CDR2 and CDR3 of one of any one of VHH1, VHH3, VHH5, VHH8, VHH10, VHH12, VHH13, VHH14, VHH15 or VHH16 shown in the Table of CDRs. (As can be seen e.g. from Example 3, Table 1, for compounds with such a VHH antibody domain a KD in the range of E-10 or stronger was observed for binding to NKp30.)

In some embodiments, in (a) said VHH antibody domain or fragment thereof comprises the complementarity determining regions CDR1, CDR2 and CDR3 of one of VHH15 shown in the Table of CDRs. (As can be seen e.g. from Example 3, Table 1, for compounds with such a VHH antibody domain a KD in the range of E-12 or stronger and a k_{off} in the range of E-07 or lower was observed for binding to NKp30.)

In some embodiments, in (a) said VHH antibody domain or fragment thereof comprises the complementarity determining regions CDR1, CDR2 and CDR3 of one of VHH1, VHH2, VHH3, VHH5, VHH6, VHH8, VHH9, VHH10, VHH11, VHH12, VHH13, VHH14, VHH15 or VHH16 shown in the Table of CDRs. (As can be seen e.g. from Example 3, Table 1, for compounds with such a VHH antibody domain a kₒₙ in the range of E-05 was observed for binding to NKp30.)

In some embodiments, in (a) said VHH antibody domain or fragment thereof comprises the complementarity determining regions CDR1, CDR2 and CDR3 of one of VHH1, VHH2, VHH3, VHH4, VHH5, VHH8, VHH10, VHH11, VHH12, VHH13, VHH14, VHH15 or VHH16 shown in the Table of CDRs. (As can be seen e.g. from Example 3, Table 1, for compounds with such a VHH antibody domain a k_{off} in the range of E-04 or lower was observed for binding to NKp30.)

In some embodiments, in (a) said VHH antibody domain or fragment thereof comprises the complementarity determining regions CDR1, CDR2 and CDR3 of one of VHH10, VHH15 or VHH16 shown in the Table of CDRs. (As can be seen e.g. from Example 3, Table 1, for compounds with such a VHH antibody domain a k_{off} in the range of E-05 or lower was observed for binding to NKp30.)

In some embodiments, in (a) said VHH antibody domain or fragment thereof comprises the complementarity determining regions CDR1, CDR2 and CDR3 of one of VHH1, VHH3, VHH5, VHH12, VHH13, VHH14, VHH15 or VHH16 shown in the Table of CDRs. (As can be seen e.g. from Example 3, Table 1, for compounds with such a VHH antibody domain competition with B7-H6 is observed. As shown in Example 4, such VHH antibody domains result in compounds that mediate lysis of targeted cells with high efficiency.)

In some embodiments, in (a) said VHH antibody domain or fragment thereof comprises the complementarity determining regions CDR1, CDR2 and CDR3 of one of VHH1, VHH5 or VHH16 shown in the Table of CDRs.

In some embodiments, in (a) said VHH antibody domain or fragment thereof comprises the complementarity determining regions CDR1, CDR2 and CDR3 of one of VHH2, VHH6, VHH7, VHH8, VHH9, VHH10 or VHH11 shown in the Table of CDRs. (As can be seen e.g. from Example 3, Table 1, for compounds with such a VHH antibody domain no competition with B7-H6 is observed. As shown in Example 4, such VHH antibody domains result in compounds that mediate lysis of targeted cells, but with a lower efficiency.)

In some embodiments, in (a) said VHH antibody domain or fragment thereof comprises the complementarity determining regions CDR1, CDR2 and CDR3 of one of VHH2, VHH4 or VHH8 shown in the Table of CDRs.

In some embodiments, in (a) said VHH antibody domain or fragment thereof comprises the complementarity determining regions CDR1, CDR2 and CDR3 of one of VHH2 or VHH8 shown in the Table of CDRs.

In some embodiments, in (a) said VHH antibody domain or fragment thereof comprises the complementarity determining regions CDR1, CDR2 and CDR3 of VHH4 shown in the Table of CDRs. (As can be seen e.g. from Example 3, Table 1, for compounds with such a VHH antibody domain partial competition with B7-H6 is observed.)

In some embodiments, in (a) said VHH antibody domain or fragment thereof comprises the complementarity determining regions CDR1, CDR2 and CDR3 of one of VHH12 or VHH15 shown in the Table of CDRs. (As can be seen e.g. from Example 3, for compounds with such a VHH antibody domain no competition with the partial competitor VHH4 is observed.)

In some embodiments, in (a) said VHH antibody domain or fragment thereof comprises the complementarity determining regions CDR1, CDR2 and CDR3 of one of VHH8, VHH9, VHH10 or VHH11 shown in the Table of CDRs. (As can be seen e.g. from Example 3, compounds with such a VHH antibody domain belong to a specific epitope bin with common characteristics.)

In some embodiments, in (a) said VHH antibody domain or fragment thereof comprises the complementarity determining regions CDR1, CDR2 and CDR3 of one of VHH2 or VHH6 shown in the Table of CDRs. (As can be seen e.g. from Example 3, compounds with such a VHH antibody domain belong to a specific epitope bin with common characteristics.)

In some embodiments, in (a) said VHH antibody domain or fragment thereof comprises the complementarity determining regions CDR1, CDR2 and CDR3 of one of VHH2, VHH6 or VHH7 shown in the Table of CDRs. (As can be seen e.g. from Example 3, compounds with such a VHH antibody domain are unique with respect to their epitope targeting.)

In some embodiments, said compound is a molecule.

In some embodiments, said compound comprises or is a protein.

By stating that the compound “comprises” a protein, the present disclosure designates that the compound includes a part within its chemical structure that is a protein. A compound that comprises a protein may or may not comprise a part that is not a protein.

In some embodiments, said compound is a protein.

By stating that the compound “is” a protein, the present disclosure designates that the compound consists only of protein and does not comprise a part that is not a protein.

In some embodiments, said VHH antibody domain is derived from a Camelid antibody. In some embodiments, said VHH antibody domain is derived from a Llama antibody.

In some embodiments, said compound comprises a VHH antibody domain or fragment thereof according to (A), (B) or (C).

In some embodiments, said compound comprises a VHH antibody domain or fragment thereof according to (A), (B) or (D).

In some embodiments, said compound comprises a VHH antibody domain or fragment thereof according to (A), (C) or (D).

In some embodiments, said compound comprises a VHH antibody domain or fragment thereof according to (A) or (B).

In some embodiments, said compound comprises a VHH antibody domain or fragment thereof according to (A) or (C).

In some embodiments, said compound comprises a VHH antibody domain or fragment thereof according to (A) or (D).

In some embodiments, said compound comprises a VHH antibody domain or fragment thereof according to (A).

In some embodiments, said compound comprises a VHH antibody domain or fragment thereof according to (B).

In some embodiments, said VHH antibody domain or fragment thereof comprises complementarity determining regions according to (a) or (b).

In some embodiments, said VHH antibody domain or fragment thereof comprises complementarity determining regions according to (a) or (c).

In some embodiments, said VHH antibody domain or fragment thereof comprises complementarity determining regions according to (a).

In some embodiments, wherein said VHH antibody domain or fragment thereof comprises complementarity determining regions according to (b).

In some embodiments, said compound further comprises a targeting moiety.

As used herein, the term “targeting moiety” refers to a moiety (i.e. a molecular group or chemical structure) that is (typically covalently) associated with said compound and that binds a target site, wherein said binding allows to recruit the compound to said target site. The target site will typically be a biological molecule or a certain part of a biological molecule. An example of a targeting moiety is an antigen-binding antibody fragment that is covalently linked to an NKp30-binding VHH antibody domain to form a compound according to the present disclosure, wherein the antigen-binding fragment binds to a certain receptor present at the surface of a certain cell type (its antigen), and wherein binding of the antigen-binding fragment to this receptor results in recruitment of the compound to this cell type.

Non-targeted drugs typically reach their site of action by whole-body distribution and passive diffusion. In contrast, targeted compounds are not distributed evenly across the whole body. Due to the interaction of targeting moiety with its target molecule, a compound including a targeting moiety is concentrated preferentially at its site target site. Therefore, e.g. therapeutic compounds with a targeting moiety require lower dosages to be therapeutically effective, thus improving the therapeutic window.

In some embodiments, all components of said compound are covalently linked.

In some embodiments, said targeting moiety is a molecular group that specifically binds to a target molecule or fragment thereof.

In some embodiments, said target molecule is a receptor at the surface of a cell.

In some embodiments, said target molecule is an antigen that is present on the surface of a target cell.

As used herein, a “target molecule that is present on the surface of a target cell” is a molecule that is present on the surface of the target cell in such a manner that it is accessible from the extracellular environment (i.e. e.g. an antibody can bind to it from the extracellular environment). For example, CD8 is a transmembrane protein of cytotoxic T cells, and its extracellular domain is accessible for antibodies directed against the extracellular domain of CD8 from the extracellular environment. Thus, in the sense of the present disclosure, CD8 is a target molecule that is present on the surface of cytotoxic T cells.

A targeting moiety that “binds” a target molecule of interest is a targeting moiety that is capable of binding that target molecule with sufficient affinity such that the targeting moiety is useful in targeting the compound to a cell expressing the target molecule.

If the present disclosure refers to a first molecule/molecular group (e.g. an antibody/antibody component) “specifically binding”/that “specifically binds” to a second molecule/molecular group (e.g. an antigen of interest), this means that the first molecule/molecular group (in this example the antibody) binds to said second molecule/molecular group (in this example the antigen of interest) with an affinity that is at least ten-fold greater than its affinity for other molecules/molecular groups, in particular other molecule/molecular group in the human body (in this example at least ten-fold greater than its affinity for binding to non-specific antigens (e.g., BSA, casein) other than said antigen of interest (or closely related antigens)). In a preferred embodiment, a first molecule/molecular group (e.g. an antibody/antibody component) that “specifically binds” to a second molecule/molecular group (e.g. an antigen of interest) binds to said antigen with an affinity that is at least 100-fold greater than its affinity for other molecules/molecular groups, in particular other molecule/molecular group in the human body (in this example at least 100-fold greater than its affinity for binding to non-specific antigens other than said antigen of interest (or closely related antigens)). Typically said binding will be determined under physiological conditions. A first molecule/molecular group that “specifically binds” to a second molecule/molecular group may bind to that second molecule/molecular group with an affinity of at least about 1×10⁷ M⁻¹.

In some embodiments, said targeting moiety is a protein, a peptide, a peptide mimetic, a nucleic acid, an oligonucleotide or a small molecule.

As used herein, the term “peptide mimetic” refers to a peptide-like chain which is designed to mimic a peptide. An example of a peptide mimetic is a D-peptide mimetic containing a D-amino acid, but is not limited thereto.

As used herein, a “small molecule”, is a molecule with a molecular weight <1000 Da.

In some embodiments, said targeting moiety comprises or is a protein.

In some embodiments, said targeting moiety comprises a protein.

In some embodiments, said targeting moiety is a protein.

In some embodiments, said targeting moiety is a protein ligand that specifically binds to a receptor at the surface of a cell.

In some embodiments, said targeting moiety is an antibody or an antigen-binding fragment thereof.

In some embodiments, said targeting moiety is capable of specifically binding to a tumor-associated antigen.

In some embodiments, said compound is a bispecific antibody. If the present disclosure states that the compound is a bispecific antibody, that does not exclude the possibility that said bispecific antibody is linked to further domains or moieties.

In some embodiments, said compound is a bispecific antibody prepared by the SEED (strand-exchange engineered domain) technology.

The term “bispecific antibody”, as used in the present disclosure, refers to an antibody that is capable of specifically binding to two different epitopes at the same time.

The terms “epitope” or “antigenic determinant” are used interchangeably herein and refer to the portion of an antigen that is recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids are typically retained upon protein denaturing, whereas epitopes formed by tertiary folding are typically lost upon protein denaturing. An epitope typically includes at least 3, and more usually, at least 5 or 8-10 amino acids in a unique spatial conformation.

The two different epitopes to which a bispecific antibody binds can be from the same antigen or from two different antigens. Preferably, the two epitopes are from two different antigens. Methods for making bispecific antibodies are known in the art. For example, bispecific antibodies can be produced recombinantly using the co-expression of two immunoglobulin heavy chain/light chain pairs (see e.g. Milstein et al., Nature (1983), vol. 305, p. 537-539). Alternatively, bispecific antibodies can be prepared using chemical linkage (see e.g. Brennan et al., Science (1985), vol. 229, p. 81). A bispecific antibody can also for example be prepared by the SEED technology (an approach for generation of bispecific antibodies in which structurally related sequences within the conserved CH3 domains of human IgA and IgG are exchanged to form two asymmetric but complementary domains, see WO 2016/087650). See the Examples section for further details.

In some embodiments, one binding site of said bispecific antibody is formed by said VHH antibody domain or fragment thereof and one binding site of said bispecific antibody is formed by said targeting moiety.

In some embodiments, said antigen-binding fragment is selected from the group consisting of a Fab, a Fab′, a (Fab′)2, a Fv, a scFv, a diabody and a VHH.

“Fab” fragments are obtained by papain digestion of an antibody, which produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site.

“F(ab′)2” fragments are obtained by pepsin treatment of an antibody, which yields a single large F(ab′)2 fragment which roughly corresponds to two disulfide linked Fab fragments having different antigen-binding activity and is still capable of cross-linking antigen.

“Fab′” fragments differ from Fab fragments by having a few additional residues at the carboxy terminus of the CH1 domain including one or more cysteines from the antibody hinge region. Fab′-SH is the designation for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and—binding site. This fragment consists of a dimer of one heavy—and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv”, also abbreviated as “scFv”, are antibody fragments that comprise the VH and VL antibody domains connected into a single polypeptide chain. Preferably, the scFv polypeptide further comprises a polypeptide linker between the VH and VL domains which enables the scFv to form the desired structure for antigen binding. For a review of the scFv, see Pluckthun, in: The Pharmacology of Monoclonal Antibodies, vol. 113 (1994), editors Rosenburg and Moore, Springer-Verlag (New York), p. 269-315.

The term “diabody” refers to a small antibody fragment prepared by constructing scFv fragments (see preceding paragraph) with short linkers (about 5-25 residues) between the VH and VL domains such that inter-chain but not intra-chain pairing of the V domains is achieved, thereby resulting in a bivalent fragment, i.e., a fragment having two antigen-binding sites. Bispecific diabodies are heterodimers of two “crossover” scFv fragments in which the VH and VL domains of the two antibodies are present on different polypeptide chains. Diabodies are described in greater detail in, for example, EP 0404097; WO 93/11161; Hollinger et al., Proc. Natl. Acad. Sci. USA (1993), vol. 90, p. 6444-6448.

In some embodiments, said antigen-binding fragment is selected from the group consisting of a Fab, a Fab′, a (Fab′)2 and a Fv.

In some embodiments, said antigen-binding fragment is a Fab.

In some embodiments, said antigen-binding fragment is selected from the group consisting of a scFv, a diabody and a VHH.

In some embodiments, said antigen-binding fragment is an antigen-binding fragment of an antibody with the SEED (strand-exchange engineered domain) format.

In some embodiments, said targeting moiety is capable of specifically binding to an antigen that is present on the surface of a target cell.

In some embodiments, said antibody is an antibody against an antigen that is present on the surface of a target cell.

In some embodiments, said antigen-binding fragment is an antigen-binding fragment of an antibody against an antigen that is present on the surface of a target cell.

An antibody/antigen-binding fragment “against” a certain antigen is an antibody/antigen-binding fragment with an antigen-binding site that binds to said antigen. If an antibody/antigen-binding fragment binds to an antigen can e.g. be determined by testing in an immunofluorescence experiment with cultured cells whether the antibody binds to cells that express the antigen at their cell surface.

In some embodiments, said antigen that is present on the surface of said target cell is more abundant on the surface of said target cell than on the surface of other cell types.

The abundance of a surface antigen on a cell type can be determined by standard methods known to a skilled person, e.g. flow cytometry (e.g. by exposing cell of said cell type to the antibody of interest, subsequently staining with a fluorescently labelled secondary antibody directed against the antibody of interest, and detection of fluorescent label by flow cytometry).

In some embodiments, said antigen that is present on the surface of said target cell is present on the surface of said target cell, but substantially not on the surface of other cell types.

As used herein, an antigen that is “present on the surface of said target cell, but substantially not on the surface of other cell types” is sufficiently abundant at the surface of the target cell to allow for recruitment of a compound with a targeting moiety (an antibody or antigen-binding fragment thereof) against said antigen under physiological conditions. In contrast, abundance of said antigen at the surface of other cell types is so low that recruitment of said compound under physiological conditions is barely above background binding.

In some embodiments, said antigen that is present on the surface of said target cell is present on the surface of said target cell, but not on the surface of other cell types.

As used herein, an antigen that is “present on the surface of said target cell, but not on the surface of other cell types” is sufficiently abundant at the surface of the target cell to allow for recruitment of a compound with a targeting moiety (an antibody or antigen-binding fragment thereof) against said antigen under physiological conditions. In contrast, abundance of said antigen at the surface of other cell types is so low that recruitment of said compound under physiological conditions is not above background binding.

In some embodiments, said binding of said targeting moiety to said antigen that is present on the surface of said target cell allows to recruit the compound specifically to said target cell.

The term “allows to recruit the antibody-drug conjugate specifically to said target cell” means that the compound is recruited to said target cell under physiological conditions with an efficiency that is at least 10 times higher, preferably at least 100 times higher, than the recruitment to other cell types (i.e. to other cell types to which said compound may be exposed in the body during administration of said compound).

In some embodiments, said antigen that is present on the surface of said target cell is a tumor-associated antigen.

As used herein, a “tumor-associated antigen” is, in its broadest sense, an antigen that allows recruitment of an ADC to the site of a tumor, such that a therapeutic action or diagnostic (e.g. labelling of the tumor site) can be achieved. The tumor-associated antigen may either be an antigen that is present on the surface of the tumor cells or an antigen associated with the tumor microenvironment.

Sources for information on cell surface expression and methods to identify and verify tumor-associated antigens are known to a skilled person and described in the literature (see e.g. Bornstein, AAPS J. (2015), vol. 17(3), p. 525-534; Hong et al., BMC Syst Biol. (2018), vol. 12 (Suppl 2), p. 17; Immune Epitope Database and Analysis Resource (https://www.iedb.org); Cancer Cell Line Encyclopedia (https://portals.broadinstitute.org/ccle); OASIS Database (http://oasis-genomics.org/)).

In preferred embodiments, said tumor-associated antigen is an antigen that is present on the surface of a tumor cell. In these embodiments, the term “tumor-associated antigen” indicates an antigen that is present at the cell surface of a tumor cell and allows for distinction of the tumor cell over other cell types. A tumor-associated antigen may be part of a molecule (e.g. a protein) that is expressed by a tumor cell and accessible from the extracellular environment. A tumor-associated antigen may differ (i.e. qualitatively differ) from its counterpart in corresponding non-tumor cells (e.g., where the molecule is a protein by one or more amino acid residues). Alternatively, the tumor-associated antigen may be identical to its counterpart in corresponding non-tumor cells, but present on the surface of the tumor cells at a higher level than on the surface of corresponding non-tumor cells. For example, the tumor-associated antigen may be present only on the surface of the tumor cells, but not on the surface of non-tumor cells, or the tumor-associated antigen may be present on the surface of tumor cells at a higher level (e.g. at least 5-fold higher, preferably at least 100-fold higher) than on the surface of non-tumor cells. In an embodiment, the tumor-associated antigen is present on the surface of tumor cells at a level that is at least 1000-fold higher than on the surface of non-tumor cells.

In some embodiments, said targeting moiety is capable of specifically binding to a tumor-associated antigen.

In some embodiments, said tumor-associated antigen is an antigen that is present on the surface of a tumor cell.

In some embodiments, said tumor-associated antigen is EGFR (epidermal growth factor receptor).

In some embodiments, said compound is a bispecific or multispecific molecule. In some embodiments, said compound is a bispecific molecule. In some embodiments, said compound is a multispecific molecule. In some embodiments, said compound is a bispecific or multispecific molecule that binds via its VHH antibody domain or fragment thereof to NKp30 and via its targeting domain to a tumor-associated antigen.

In some embodiments, said compound comprises an antibody Fc region.

As used herein, the term “antibody Fc region” refers to the portion of a native immunoglobulin formed by the Fc domains of its two heavy chains (which includes a heavy chain constant region 1 (CH1), a heavy chain constant region 2 (CH2) and a heavy chain constant region 3 (CH3) of an immunoglobulin, but does not include variable regions of the heavy and light chains and a light chain constant region 1 (CL1) of an immunoglobulin). A native Fc region is homodimeric. In some embodiments, the term includes variant Fc regions with one or more alterations relative to a native Fc region. An Fc region may be altered by amino acid substitutions, additions and/or deletions, linkage of additional moieties, and/or alteration of the native glycans. The term encompasses Fc regions wherein each of the constituent Fc domains is different. Examples of heterodimeric Fc regions include, without limitation, Fc regions made using the “knobs into holes” technology as described in, for example U.S. Pat. No. 8,216,805 or by the SEED technology as described in WO 2016/087650.

In some embodiments, said compound comprises an antibody Fc region competent in Fc receptor binding.

An antibody Fc region is “competent in FC receptor binding” if said antibody Fc region is capable of binding to at least one of the Fc receptors (FcγRI, FcγRII, and FcγRIII subclasses, including allelic variants and alternatively spliced forms of these receptors).

In some embodiments, said compound comprises an antibody Fc region that is not competent in Fc receptor binding.

In some embodiments, said compound does not comprise an effector-competent antibody Fc region.

An “effector-competent” Fc region is an Fc region having the functional ability to bind proteins and/or cells of the immune system and mediate biological effects normally induced following the binding of an antibody to a corresponding antigen. Such biological effects include e.g. the ability to bind a complement protein (e.g. C1q), resulting in activation of the classical complement system leading to the opsonisation and lysis of cell pathogens (complement-dependent cytotoxicity, CDCC). Other biological effects are endocytosis of immune complexes, engulfment and destruction of antibody-coated particles or microorganisms (also called antibody-dependent phagocytosis, or ADCP), clearance of immune complexes, lysis of antibody-coated target cells by killer cells (called antibody-dependent cell-mediated cytotoxicity, or ADCC), release of inflammatory mediators, regulation of immune system cell activation or control of immunoglobulin production.

In some embodiments, said compound comprises an effector-competent antibody Fc region.

In some embodiments, said compound does not comprise an antibody Fc region capable of inducing ADCC (antibody-dependent cellular cytotoxicity).

In some embodiments, said compound comprises an antibody Fc region capable of inducing ADCC.

In some embodiments, said compound comprises an antibody Fc region that is not capable of inducing ADCC.

In some embodiments, said compound is capable of inducing both FcγRIIIa signalling and positive (i.e. NK cell activating) NKp30 signalling.

In some embodiments, said compound is capable of specifically binding to NKp30 on NK cells.

In some embodiments, said compound is capable of activating NK cells by binding to NKp30 on said NK cells.

Whether a compound or domain is, upon binding to NKp30 on NK cells, capable of activating NK cells can be determined as described in Example 1, section “NK cell activation assay”. If this activation occurs upon binding to NKp30 on NK cells can for example be determined by carrying out a control experiment with NK cells in which NKp30 has been blocked by a competitor molecule that binds to NKp30 such that the protein domain cannot get access to NKp30 on the NK cells.

Preferably, binding to NKp30 and activation of NK cells is assessed with said VHH antibody domain in the context of the complete compound.

In some embodiments, binding of said compound to NKp30 on NK cells activates said NK cells.

In some embodiments, said compound is an agonist of NKp30.

In some embodiments, said compound is, upon binding of said VHH antibody domain or fragment thereof to NKp30 on NK cells, capable of activating NK cells.

In some embodiments, binding of said compound to NKp30 on NK cells activates said NK cells.

In some embodiments, activation of NK cells is determined by measuring expression of the activation marker CD69 by flow cytometry.

In some embodiments, said compound shows cytotoxic activity in a ⁵¹Cr release assay.

In some embodiments, said ⁵¹Cr release assay is carried out as a 4 h ⁵¹Cr release assay, wherein human PBMCs are used as effector cells at effector-to-target cell (E:T) ratios of 80:1, and wherein a higher percent lysis indicates improved cytotoxic activity.

In some embodiments, said ⁵¹Cr release assay is carried out as described in Repp et al., 2011.

As used herein, the reference “Repp et al., 2011” refers to the publication R. Repp et al., “Combined Fc-protein- and Fc-glyco-engineering of scFv-Fc fusion proteins synergistically enhances CD16a binding but does not further enhance NK-cell mediated ADCC,” Journal of Immunological Methods (2011), vol. 373, p. 67-78.

In some embodiments, binding of said compound to NKp30 on NK cells results in the release of interferon-γ (IFN-γ).

In some embodiments, said release of IFN-γ is measured as follows: isolated human NK cells are incubated overnight in medium containing 100 U/ml recombinant human interleukin-2, in different wells A431 cells are seeded and incubated for 3 h, the compound is added to a final concentration of 85 nM followed by addition of NK cells at an E:T ratio of 5:1, human IFN-γ is analyzed in the culture supernatant after 24 h by ELISA.

In some embodiments, binding of said compound to NKp30 on NK cells results in the release of tumor necrosis factor-α (TNF-α).

In some embodiments, said release of TNF-α is measured as follows: isolated human NK cells are incubated overnight in medium containing 100 U/ml recombinant human interleukin-2, in different wells A431 cells are seeded and incubated for 3 h, the compound is added to a final concentration of 85 nM followed by addition of NK cells at an E:T ratio of 5:1, human TNF-α is analyzed in the culture supernatant after 24 h by ELISA.

According to another aspect, the present disclosure relates to a pharmaceutical composition comprising the compound according to any one of the aspects or embodiments described above.

Methods for preparing pharmaceutical compositions are known to a skilled person in the art (Remington: The Science and Practice of Pharmacy, 22nd ed. (2012), Pharmaceutical Press).

In some embodiments, said pharmaceutical composition comprises a pharmaceutically acceptable carrier, diluent and/or excipient.

The term “pharmaceutically acceptable” designates that said carrier, diluent or excipient is a non-toxic, inert material that is compatible with the other ingredients of the pharmaceutical composition and not harmful to the patient that the pharmaceutical composition is administered to, such that it can be used in a pharmaceutical product. Substances suitable as carriers, diluents or excipients in pharmaceutical compositions are known to a skilled person in the art (Remington: The Science and Practice of Pharmacy, 22nd ed. (2012), Pharmaceutical Press). The pharmaceutical composition may further include e.g. additional adjuvants, antioxidants, buffering agents, bulking agents, colorants, emulsifiers, fillers, flavoring agents, preservatives, stabilizers, suspending agents and/or other customary pharmaceutical auxiliaries.

In some embodiments, said pharmaceutical composition further includes at least one additional adjuvant, antioxidant, buffering agent, bulking agent, colorant, emulsifier, filler, flavoring agent, preservative, stabilizer, suspending agent and/or other customary pharmaceutical auxiliary.

According to another aspect, the present disclosure relates to a compound according to any of the aspects or embodiments described above or a pharmaceutical composition according to any of the aspects or embodiments described above for use as a medicament.

According to another aspect, the present disclosure relates to a compound according to any of the aspects or embodiments described above or a pharmaceutical composition according to any of the aspects or embodiments described above for use in the treatment of cancer.

According to another aspect, the present disclosure relates to a compound according to any of the aspects or embodiments described above or a pharmaceutical composition according to any of the aspects or embodiments described above for use in the treatment of a malignant tumor.

In some embodiments, said compound/said pharmaceutical composition is for use in the treatment of a human.

The production of medicaments containing the compound of the present disclosure according or a pharmaceutical composition according to the present disclosure can be performed according to well-known pharmaceutical methods. Further details on techniques for formulation and administration may be found e.g. in Remington: The Science and Practice of Pharmacy, 22nd ed. (2012), Pharmaceutical Press.

As used herein, “treatment” of a disease and “treating” a disease refers to the process of providing a subject with a pharmaceutical treatment, e.g., the administration of a drug, such that said disease is alleviated, reduced, minimized, halted or even healed, and/or such that the chances of a relapse into the disease are reduced or a relapse into the disease is even prevented.

The use of compounds in the treatment of diseases is known to a skilled person in the art (see e.g. Coats et al., Clinical Cancer Research (2019), vol. 25(18), p. 5441-5448; Rudra, Bioconjugate Chemistry (2020), vol. 31(3), p. 462-473). Thus, the skilled person is aware that the components of the compound, in particular the targeting moiety, must be selected appropriately in order to allow for successful treatment. For example, for treatment of a specific cancer, the targeting moiety of the compound must be selected such that binding of the targeting moiety to its target site directs the compound to said cancer (e.g. by using an antibody component against a tumor-associated antigen that is specifically found on the surface of the cancer cells). Cytotoxic effects will then be achieved by the affinity-matured variant B7-H6 sequence included in the compound. In addition, a payload may be included in the compound such that an additional desired treatment effect is achieved. For example, for the treatment of a cancer, a cytotoxic drug may be included in addition.

In another aspect, the present disclosure relates to a method for treating a disease in a patient in need thereof, comprising the step of administering to said patient a therapeutically effective amount of the compound according to any of the aspects or embodiments described above or the pharmaceutical composition according to any of the aspects or embodiments described above.

By “therapeutically effective amount” is meant the amount of an agent required to ameliorate the symptoms of a disease. The effective amount of active agent(s) (e.g., a compound according to the present disclosure) used for therapeutic treatment of a disease according to the present disclosure varies depending upon the manner of administration, the age, body weight, and general health of the subject. Ultimately, the attending physician or veterinarian will decide the appropriate amount and dosage regimen. Such amount is referred to as a “therapeutically effective” amount.

The term “patient”, as used herein, refers to a mammal (such as a human, rat, mouse, monkey, pig, goat, cow, horse, dog or cat). Preferably, the patient is a human.

In some embodiments, said disease is cancer.

In some embodiments, said disease is a malignant tumor.

As used herein, the term “cancer” refers to a malignant neoplasm. Cancer can include a hematological cancer or a solid tumor. For example, the cancer can be a leukemia (e.g., acute myeloid leukemia (AML), acute monocytic leukemia, promyelocytic leukemia, eosinophilic leukaemia, acute lymphoblastic leukemia (ALL) such as acute B lymphoblastic leukemia (B-ALL), chronic myelogenous leukemia (CML), chronic lymphocytic leukemia (CLL)) or lymphoma (e.g., non-Hodgkin lymphoma), myelodysplastic syndrome (MDS)” melanoma, lung cancer (e.g., non-small cell lung cancer; NSCLC), ovarian cancer, endometrial cancer, peritoneal cancer, pancreatic cancer, breast cancer, prostate cancer, squamous cell carcinoma of the head and neck, or cervical cancer. Preferably, in the present disclosure the term “cancer” refers to a solid malignant tumor.

In some embodiments, said patient is a human.

In another aspect, the present disclosure relates to the use of the compound according to any of the aspects or embodiments described above or of the pharmaceutical composition according to any of the aspects or embodiments described above for the manufacture of a medicament.

In another aspect, the present disclosure relates to the use of the compound according to any of the aspects or embodiments described above or of the pharmaceutical composition according to any of the aspects or embodiments described above for the manufacture of a medicament for the treatment of cancer.

In another aspect, the present disclosure relates to the use of the compound according to any of the aspects or embodiments described above or of the pharmaceutical composition according to any of the aspects or embodiments described above for the manufacture of a medicament for the treatment of a malignant tumor.

In some embodiments, said medicament is prepared for administration to a human.

The following embodiments relate to any of the compounds or pharmaceutical compositions for use in medical treatment, methods for treating a disease in a patient in need thereof, uses for the manufacture of a medicament, or any of their embodiments described above.

In some embodiments, said cancer or malignant tumor is a human disease.

The following embodiments relate to any of the compounds, pharmaceutical compositions, compounds or pharmaceutical compositions for use in medical treatment, methods for treating a disease in a patient in need thereof, uses for the manufacture of a medicament, or any of their embodiments described above.

In some embodiments, said compound comprises the sequence represented by SEQ ID NO: 1 (or a fragment thereof), with one or more amino acid substitutions compared to the sequence of SEQ ID NO: 1, wherein, if such amino acid substitutions are located outside of the CDRs, said amino acid substitution(s) are selected from the following: D1E, Q5V, L11V, T24A, E44G, H45L, G47W, F70I, V79L, K87R, P88A, Q116L.

In some embodiments, said compound comprises the sequence represented by SEQ ID NO: 1 (or a fragment thereof), with at least the following amino acid substitutions: D1E, Q5V, L11V, V79L, K87R, P88A, Q116L; and with optionally one or more further amino acid substitutions compared to the sequence of SEQ ID NO: 1, wherein, if such further amino acid substitutions are located outside of the CDRs, such further amino acid substitution(s) are selected from the following: T24A, E44G, H45L, F70I.

In some embodiments, said compound comprises the sequence represented by SEQ ID NO: 1 (or a fragment thereof), with the additional modification of (b) according to any one of items [1] to or with the additional modification of (c) according to any one of items [1] or to [22].

In some embodiments, said compound comprises the sequence represented by SEQ ID NO: 1 (or a fragment thereof), with the additional modification of (b) according to any one of items [1] to or with the additional modification of (c) according to any one of items [1] or to [22]; and with one or more further amino acid substitutions compared to the sequence of SEQ ID NO: 1, wherein said amino acid substitution(s) are selected from the following: D1E, Q5V, L11V, T24A, E44G, H45L, G47W, F70I, V79L, K87R, P88A, Q116L.

In some embodiments, said compound comprises the sequence represented by SEQ ID NO: 1 (or a fragment thereof), with the additional modification of (b) according to any one of items [1] to or with the additional modification of (c) according to any one of items [1] or to [22]; and the amino acid substitutions D1E, Q5V, L11V, V79L, K87R, P88A, Q116L; and with optionally one or more further amino acid substitutions compared to the sequence of SEQ ID NO: 1, wherein said further amino acid substitution(s) are selected from the following: T24A, E44G, H45L, F70I.

In some embodiments, said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or a fragment thereof.

In some embodiments, said compound has a sequence represented by any one of the following SEQ ID NOs: SEQ ID NO: 67, SEQ ID NO: 68, SEQ ID NO: 69, or a fragment thereof.

In some embodiments, said compound has a sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 77, or a fragment thereof.

In some embodiments, said compound has a sequence represented by any one of the following SEQ ID NOs: SEQ ID NO: 67, SEQ ID NO: 68, SEQ ID NO: 69, SEQ ID NO: 77, or a fragment thereof.

In some embodiments, said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 78, or a fragment thereof.

In some embodiments, said compound comprises the sequence represented by any one of the following SEQ ID NOs: SEQ ID NO: 67, SEQ ID NO: 68, SEQ ID NO: 69, SEQ ID NO: 78, or a fragment thereof.

In some embodiments, said compound comprises the sequence defined by SEQ ID NO: 77, or a fragment thereof.

In some embodiments, said compound comprises the sequence defined by SEQ ID NO: 78, or a fragment thereof.

In some embodiments, said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or a fragment thereof; with the additional modification of (b) according to any one of items [1] to or with the additional modification of (c) according to any one of items [1] or to [22].

In some embodiments, said compound comprises the sequence represented by any one of the following SEQ ID NOs: SEQ ID NO: 67, SEQ ID NO: 68, SEQ ID NO: 69, or a fragment thereof; with the additional modification of (b) according to any one of items [1] to or with the additional modification of (c) according to any one of items [1] or to [22].

In some embodiments, said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 77, or a fragment thereof; with the additional modification of (b) according to any one of items [1] to or with the additional modification of (c) according to any one of items [1] or to [22].

In some embodiments, said compound comprises the sequence represented by any one of the following SEQ ID NOs: SEQ ID NO: 67, SEQ ID NO: 68, SEQ ID NO: 69, SEQ ID NO: 77, or a fragment thereof; with the additional modification of (b) according to any one of items [1] to or with the additional modification of (c) according to any one of items [1] or to [22].

In some embodiments, said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 78, or a fragment thereof; with the additional modification of (b) according to any one of items [1] to or with the additional modification of (c) according to any one of items [1] or to [22].

In some embodiments, said compound comprises the sequence represented by any one of the following SEQ ID NOs: SEQ ID NO: 67, SEQ ID NO: 68, SEQ ID NO: 69, SEQ ID NO: 78, or a fragment thereof; with the additional modification of (b) according to any one of items [1] to or with the additional modification of (c) according to any one of items [1] or to [22].

In some embodiments, said compound comprises the sequence defined by SEQ ID NO: 77 or a fragment thereof, with the additional modification of (b) according to any one of items [1] to or with the additional modification of (c) according to any one of items [1] or to [22].

In some embodiments, said compound comprises the sequence defined by SEQ ID NO: 78 or a fragment thereof, with the additional modification of (b) according to any one of items [1] to or with the additional modification of (c) according to any one of items [1] or to [22].

In some embodiments, said compound comprises the sequence represented by SEQ ID NO: 2 (or a fragment thereof), with one or more amino acid substitutions compared to the sequence of SEQ ID NO: 2, wherein, if such amino acid substitutions are located outside of the CDRs, said amino acid substitution(s) are selected from the following: D1E, Q5V, L11V, A14P, V24A, F37V, A44G, R45L, S47W, V79L, K87R, P88A, Q118L.

In some embodiments, said compound comprises the sequence represented by SEQ ID NO: 2 (or a fragment thereof), with the amino acid substitutions D1E, Q5V, L11V, K87R, P88A, Q118L; and with optionally one or more further amino acid substitutions compared to the sequence of SEQ ID NO: 2, wherein, if such further amino acid substitutions are located outside of the CDRs, such further amino acid substitution(s) are selected from the following: A14P, V24A, A44G, R45L, S47W, V79L.

In some embodiments, said compound comprises the sequence represented by SEQ ID NO: 2 (or a fragment thereof), with the amino acid substitutions D1E, Q5V, L11V, A14P, V24A, K87R, P88A, Q118L; and with optionally one or more further amino acid substitutions compared to the sequence of SEQ ID NO: 2, wherein, if such further amino acid substitutions are located outside of the CDRs, such further amino acid substitution(s) are selected from the following: A44G, R45L, S47W, V79L.

In some embodiments, said compound comprises the sequence represented by SEQ ID NO: 2 (or a fragment thereof), with the additional modification of (b) according to any one of items [1] to or with the additional modification of (c) according to any one of items [1] or to [22].

In some embodiments, said compound comprises the sequence represented by SEQ ID NO: 2 (or a fragment thereof), with the additional modification of (b) according to any one of items [1] to or with the additional modification of (c) according to any one of items [1] or to [22]; and with one or more further amino acid substitutions compared to the sequence of SEQ ID NO: 2, wherein said amino acid substitution(s) are selected from the following: D1E, Q5V, L11V, A14P, V24A, F37V, A44G, R45L, S47W, V79L, K87R, P88A, Q118L.

In some embodiments, said compound comprises the sequence represented by SEQ ID NO: 2 (or a fragment thereof), with the additional modification of (b) according to any one of items [1] to or with the additional modification of (c) according to any one of items [1] or to [22]; and with the amino acid substitutions D1E, Q5V, L11V, K87R, P88A, Q118L; and with optionally one or more further amino acid substitutions compared to the sequence of SEQ ID NO: 2, wherein said further amino acid substitution(s) are selected from the following: A14P, V24A, A44G, R45L, S47W, V79L.

In some embodiments, said compound comprises the sequence represented by SEQ ID NO: 2 (or a fragment thereof), with the additional modification of (b) according to any one of items [1] to or with the additional modification of (c) according to any one of items [1] or to [22]; and with the amino acid substitutions D1E, Q5V, L11V, A14P, V24A, K87R, P88A, Q118L; and with optionally one or more further amino acid substitutions compared to the sequence of SEQ ID NO: 2, wherein said further amino acid substitution(s) are selected from the following: A44G, R45L, S47W, V79L.

In some embodiments, said compound comprises the sequence represented by any one of the following SEQ ID NOs: SEQ ID NO: 79, SEQ ID NO: 80, SEQ ID NO: 81, SEQ ID NO: 83, SEQ ID NO: 84, SEQ ID NO: 85, SEQ ID NO: 86, SEQ ID NO: 89, SEQ ID NO: 90, SEQ ID NO: 93, SEQ ID NO: 94, or a fragment thereof.

In some embodiments, said compound comprises the sequence represented by any one of the following SEQ ID NOs: SEQ ID NO: 79, SEQ ID NO: 80, SEQ ID NO: 83, SEQ ID NO: 86, SEQ ID NO: 89, SEQ ID NO: 90, SEQ ID NO: 94, or a fragment thereof.

In some embodiments, said compound comprises the sequence represented by any one of the following SEQ ID NOs: SEQ ID NO: 79, SEQ ID NO: 80, SEQ ID NO: 81, SEQ ID NO: 83, SEQ ID NO: 84, SEQ ID NO: 85, SEQ ID NO: 86, SEQ ID NO: 89, SEQ ID NO: 90, SEQ ID NO: 93, SEQ ID NO: 94, or a fragment thereof; with the additional modification of (b) according to any one of items [1] to or with the additional modification of (c) according to any one of items [1] or to [22].

In some embodiments, said compound comprises the sequence represented by any one of the following SEQ ID NOs: SEQ-ID SEQ ID NO: 79, SEQ ID NO: 80, SEQ ID NO: 83, SEQ ID NO: 86, SEQ ID NO: 89, SEQ ID NO: 90, SEQ ID NO: 94, or a fragment thereof; with the additional modification of (b) according to any one of items [1] to or with the additional modification of (c) according to any one of items [1] or to [22].

In some embodiments, said VHH antibody domain comprises the VHH sequence VHH1 shown in the Table of VHH Sequences or a fragment thereof, with additional modification of (B) or (C), wherein said modification of (B) or (C) consists of the substitution of one or more amino acids compared to the sequence of SEQ ID NO: 1, wherein said amino acid substitution(s) are selected from the following: D1E, Q5V, L11V, T24A, E44G, H45L, G47W, F70I, V79L, K87R, P88A, Q116L.

In some embodiments, said VHH antibody domain comprises the VHH sequence VHH1 shown in the Table of VHH Sequences or a fragment thereof, with additional modification of (B) or (C), wherein said modification of (B) or (C) consists of the amino acid substitutions D1E, Q5V, L11V, V79L, K87R, P88A, Q116L compared to the sequence of SEQ ID NO: 1; and optionally one or more further amino acid substitutions compared to the sequence of SEQ ID NO: 1, wherein said further amino acid substitution(s) are selected from the following: T24A, E44G, H45L, F70I.

In some embodiments, said VHH antibody domain comprises a VHH sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or a fragment thereof.

In some embodiments, said VHH antibody domain comprises a VHH sequence represented by any one of the following SEQ ID NOs: SEQ ID NO: 67, SEQ ID NO: 68, SEQ ID NO: 69, or a fragment thereof.

In some embodiments, said VHH antibody domain comprises a VHH sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 77, or a fragment thereof.

In some embodiments, said VHH antibody domain comprises a VHH sequence represented by any one of the following SEQ ID NOs: SEQ ID NO: 67, SEQ ID NO: 68, SEQ ID NO: 69, SEQ ID NO: 77, or a fragment thereof.

In some embodiments, said VHH antibody domain comprises a VHH sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 78, or a fragment thereof.

In some embodiments, wherein said VHH antibody domain comprises a VHH sequence represented by any one of the following SEQ ID NOs: SEQ ID NO: 67, SEQ ID NO: 68, SEQ ID NO: 69, SEQ ID NO: 78, or a fragment thereof.

In some embodiments, said VHH antibody domain comprises the VHH sequence VHH1 shown in the Table of VHH Sequences or a fragment thereof, with additional modification of (B) or (C), wherein said modification of (B) or (C) consists of the substitution of one or more amino acids compared to the sequence of SEQ ID NO: 2, wherein said amino acid substitution(s) are selected from the following: D1E, Q5V, L11V, A14P, V24A, F37V, A44G, R45L, S47W, V79L, K87R, P88A, Q118L.

In some embodiments, said VHH antibody domain comprises the VHH sequence VHH1 shown in the Table of VHH Sequences or a fragment thereof, with additional modification of (B) or (C), wherein said modification of (B) or (C) consists of the amino acid substitutions D1E, Q5V, L11V, K87R, P88A, Q118L compared to the sequence of SEQ ID NO: 2; and optionally one or more further amino acid substitutions compared to the sequence of SEQ ID NO: 2, wherein said further amino acid substitution(s) are selected from the following: A14P, V24A, A44G, R45L, S47W, V79L.

In some embodiments, said VHH antibody domain comprises the VHH sequence VHH1 shown in the Table of VHH Sequences or a fragment thereof, with additional modification of (B) or (C), wherein said modification of (B) or (C) consists of the amino acid substitutions D1E, Q5V, L11V, A14P, V24A, K87R, P88A, Q118L compared to the sequence of SEQ ID NO: 2; and optionally one or more further amino acid substitutions compared to the sequence of SEQ ID NO: 2, wherein said further amino acid substitution(s) are selected from the following: A44G, R45L, S47W, V79L.

In some embodiments, said VHH antibody domain comprises a VHH sequence represented by any one of the following SEQ ID NOs: SEQ ID NO: 79, SEQ ID NO: 80, SEQ ID NO: 81, SEQ ID NO: 83, SEQ ID NO: 84, SEQ ID NO: 85, SEQ ID NO: 86, SEQ ID NO: 89, SEQ ID NO: 90, SEQ ID NO: 93, SEQ ID NO: 94, or a fragment thereof.

In some embodiments, said VHH antibody domain comprises a VHH sequence represented by any one of the following SEQ ID NOs: SEQ ID NO: 79, SEQ ID NO: 80, SEQ ID NO: 83, SEQ ID NO: 86, SEQ ID NO: 89, SEQ ID NO: 90, SEQ ID NO: 94, or a fragment thereof.

In some embodiments, said compound binds to NKp30 with an affinity of at least about 1×10⁶ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).

In some embodiments, said compound binds to NKp30 with an affinity of at least about 5×10⁶ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).

In some embodiments, said compound binds to NKp30 with an affinity of at least about 1×10⁷ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).

In some embodiments, said compound binds to NKp30 with an affinity of at least about 5×10⁸⁷ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).

In some embodiments, said compound binds to NKp30 with an affinity of at least about 1×10⁸ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).

Also disclosed with regard to the above-described subject matter is the following:
[1] A compound comprising a VHH antibody domain or a fragment thereof, wherein
   (a) said VHH antibody domain or fragment thereof comprises the complementarity determining regions CDR1, CDR2 and CDR3 of one of VHH1 to VHH16 as shown in the Table of CDRs below;
   (b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized; or
   (c) said VHH antibody domain or fragment thereof comprises the complementarity determining regions CDR1, CDR2 and CDR3 as defined in (a) with modification, wherein the modification is
      the replacement, addition or deletion of up to three amino acids in CDR1,
      the replacement, addition or deletion of up to three amino acids in CDR2 and/or
      the replacement, addition or deletion of up to three amino acids in CDR3;

Table of CDRs:

| | CDR1 | CDR2 | CDR3 |
|---|---|---|---|
| VHH1 | ATSGFTLDDY | CFSPS | AASFADGSSW |
| | TIG | DGTTY | CYDYW |
| | (SEQ ID | (SEQ ID | (SEQ ID |
| | NO: 18) | NO: 19) | NO: 20) |
| VHH2 | AVSGQTWTNY | SIEWGGRGTY | AAQSSSRSPL |
| | HIG | A | ESNYDYW |
| | (SEQ ID | (SEQ ID | (SEQ ID |
| | NO: 21) | NO: 22) | NO: 23) |
| VHH3 | LVSGVTYHFY | CISYASGTTK | ATQGSGSCSD |
| | DIT | E | FMSAGSVLSW |
| | (SEQ ID | (SEQ ID | (SEQ ID |
| | NO: 24) | NO: 25) | NO: 26) |
| VHH4 | LVSGVTYHFY | CISYASGSTK | AMQGSGSCSD |
| | DIT | F | EMSADSVLSW |
| | (SEQ ID | (SEQ ID | (SEQ ID |
| | NO: 27) | NO: 28) | NO: 29) |
| VHH5 | LVSGVTYHFY | CISYASGSTK | ATQGSGSCSD |
| | DIT | E | EMSADSVLSW |
| | (SEQ ID | (SEQ ID | (SEQ ID |
| | NO: 30) | NO: 31) | NO: 32) |
| VHH6 | AASGLPFSKY | VVSWSGD | AASGSYPVTD |
| | AIA | KFY | RYFYNFW |
| | (SEQ ID | (SEQ ID | (SEQ ID |
| | NO: 33) | NO: 34) | NO: 35) |
| VHH7 | AASGFTFSSY | AAIFSAGGST | NKAPPFTTIQ |
| | DMG | IYA | PTCVMIDTQL |
| | (SEQ ID | (SEQ ID | GAW |
| | NO: 36) | NO: 37) | (SEQ ID |
| | | | NO: 38) |
| VHH8 | AASGFTFSSY | AAIFSADGST | NKAPPFTTIQ |
| | DMG | IYA | PMCVMIDTQL |
| | (SEQ ID | (SEQ ID | GAW |
| | NO: 39) | NO: 40) | (SEQ ID |
| | | | NO: 41) |
| VHH9 | AASGRGGASY | AAISWTGDTT | AAAHWTRVVA |
| | AMG | YN | KGAMADEKKY |
| | (SEQ ID | (SEQ ID | EYW |
| | NO: 42) | NO: 43) | (SEQ ID |
| | | | NO: 44) |
| VHH10 | AASGRTESSN | AAIHWSGGSR | AAEISWQTTG |
| | EMG | AYA | WTNLGSW |
| | (SEQ ID | (SEQ ID | (SEQ ID |
| | NO: 45) | NO: 46) | NO: 47) |
| VHH11 | AASGRTFSSD | AAIHWSGGST | AAEIRWLTTG |
| | EMG | SYA | WANLGSW |
| | (SEQ ID | (SEQ ID | (SEQ ID |
| | NO: 48) | NO: 49) | NO: 50) |
| VHH12 | AASGRTFSSD | AAIHWSGGSR | AAEIRWQTTG |
| | EMG | SYA | WANLGSW |
| | (SEQ ID | (SEQ ID | (SEQ ID |
| | NO: 51) | NO: 52) | NO: 53) |
| VHH13 | AASGRTFTSD | AAIHWSGGSR | AAEIRWQTAG |
| | EMG | SYA | WVNLGSW |
| | (SEQ ID | (SEQ ID | (SEQ ID |
| | NO: 54) | NO: 55) | NO: 56) |
| VHH14 | EASGVTENFY | ADITSEGRTE | YTGG |
| | SMT | YA | SW |
| | (SEQ ID | (SEQ ID | (SEQ ID |
| | NO: 57) | NO: 58) | NO: 59) |
| VHH15 | AVSGSTFRLN | AGITADGGVT | NTAPA |
| | VMG | NYA | NWGSW |
| | (SEQ ID | (SEQ ID | (SEQ ID |
| | NO: 60) | NO: 61) | NO: 62) |
| VHH16 | TASGSIFSSL | AGITTDGSTD | NKSPYYDPTD |
| | TMAW | YA | YDSW |
| | (SEQ ID | (SEQ ID | (SEQ ID |
| | NO: 63) | NO: 64) | NO: 65) |

[2] The compound of item [1], wherein the modification in (b) is that the sequence of CDR1 and/or CDR2, but not the sequence of CDR3 is humanized.
[3] The compound of any one of items [1] or [2], wherein the modification in (b) is that the sequence of CDR1 is humanized, but not the sequence of CDR2 and CDR3.
[4] The compound of any one of items [1] or [2], wherein the modification in (b) is that the sequence of CDR2 is humanized, but not the sequence of CDR1 and CDR3.
[5] The compound of any one of items [1] to [4], wherein the modification in (b) is that the sequence of one, but not more than one of CDR1, CDR2 and CDR3 is humanized.
[6] The compound of any one of items [1] to [5], wherein said humanization of said CDR(s) is by replacing at least one amino acid in the sequence of said CDR by the corresponding amino acid of a human VH domain.
[7] The compound of any one of items [1] to [6], wherein said humanization of said CDR(s) is by replacing up to three amino acids in the sequence of said CDR by the corresponding amino acid of a human VH domain.
[8] The compound of any one of items [1] to [6], wherein said humanization of said CDR(s) is by replacing up to three amino acids in the sequence of CDR1 and/or CDR2 and up to one amino acid in the sequence of CDR3 by the corresponding amino acid of a human VH domain.
[9] The compound of any one of items [1] to [6], wherein said humanization of said CDR(s) is by replacing up to two amino acids in the sequence of said CDR by the corresponding amino acid of a human VH domain.
[10] The compound of any one of items [1] to [6], wherein said humanization of said CDR(s) is by replacing up to two amino acids in the sequence of CDR1 and/or CDR2 and up to one amino acid in the sequence of CDR3 by the corresponding amino acid of a human VH domain.
[11] The compound of any one of items [1] to [10], wherein said humanization of said CDR(s) is by replacing one amino acid in the sequence of said CDR by the corresponding amino acid of a human VH domain.
[12] The compound of any one of items [1] to [11], wherein the modification in (c) is
   the replacement, addition or deletion of up to three amino acids in CDR1,
   the replacement, addition or deletion of up to three amino acids in CDR2 and/or
   the replacement, addition or deletion of up to one amino acid in CDR3.
[13] The compound of any one of items [1] to [11], wherein the modification in (c) is
   the replacement, addition or deletion of up to two amino acids in CDR1,
   the replacement, addition or deletion of up to two amino acids in CDR2 and/or
   the replacement, addition or deletion of up to two amino acids in CDR3;
[14] The compound of any one of items [1] to [11], wherein the modification in (c) is
   the replacement, addition or deletion of up to two amino acids in CDR1;
   the replacement, addition or deletion of up to two amino acids in CDR2; and/or
   the replacement, addition or deletion of up to one amino acid in CDR3.
[15] The compound of any one of items [1] to [11], wherein the modification in (c) is
   the replacement, addition or deletion of up to two amino acids in CDR1 and/or
   the replacement, addition or deletion of up to two amino acids in CDR2;
wherein the sequence of CDR3 is unmodified.
[16] The compound of any one of items [1] to [11], wherein the modification in (c) is the replacement, addition or deletion of up to two amino acids in CDR1, wherein the sequence of CDR2 and CDR3 is unmodified.
[17] The compound of any one of items [1] to [11], wherein the modification in (c) is
   the replacement, addition or deletion of up to two amino acids in CDR2,
wherein the sequence of CDR1 and CDR3 is unmodified.
[18] The compound of any one of items [1] to [11], wherein the modification in (c) is
   the replacement, addition or deletion of up to one amino acid in CDR1;
   the replacement, addition or deletion of up to one amino acid in CDR2; and/or
   the replacement, addition or deletion of up to one amino acid in CDR3.
[19] The compound of any one of items [1] to [11], wherein the modification in (c) is
   the replacement, addition or deletion of one amino acid in CDR1 and/or
   the replacement, addition or deletion of one amino acid in CDR2;
wherein the sequence of CDR3 is unmodified.
[20] The compound of any one of items [1] to [11], wherein the modification in (c) is
   the replacement, addition or deletion of one amino acid in CDR1,
wherein the sequence of CDR2 and CDR3 is unmodified.
[21] The compound of any one of items [1] to [11], wherein the modification in (c) is
   the replacement, addition or deletion of one amino acid in CDR2,
wherein the sequence of CDR1 and CDR3 is unmodified.
[22] The compound of any one of items [1] to [21], wherein the modification in (c) comprises only the replacement, but not the addition or deletion of amino acids.
[23] A compound comprising a VHH antibody domain or a fragment thereof, wherein
   (A) said VHH antibody domain comprises any one of the VHH sequences VHH1 to VHH16 shown in the Table of VHH Sequences below;
   (B) said VHH antibody domain comprises a VHH sequence as defined in (A) with modification, wherein the modification is that said sequence is humanized;
   (C) said VHH antibody domain comprises a VHH sequence as defined in (A) with modification, wherein the modification is the replacement, addition or deletion of up to 25 amino acids; or
   (D) said VHH antibody domain comprises a VHH sequence that is at least 75% identical to a VHH sequence referred to in (A);

Table of VHH Sequences:

| VHH Sequence | SEQ ID NO | Amino acid sequence |
|---|---|---|
| VHH1 | SEQ ID NO: 1 | DVQLQESGGGLVQPGGSLRLSCATSGFTLDDYT |
| | | IGWVRQAPGKEHEGVSCFSPSDGTTYYADSVKG |
| | | RFTFSRDNAKNTVYLQMNSLKPEDTAVYYCAAS |
| | | FADGSSWCYDYWGQGTQVTVSS |
| VHH2 | SEQ ID NO: 2 | DVQLQESGGGLVQAGGSLRLSCAVSGQTWINYH |
| | | IGWFRQAPGKARESVASIEWGGRGTYATDSVKG |
| | | RFTISRDNAKNTVYLQMNSLKPEDTAVYYCAAQ |
| | | SSSRSPLESNYDYWGQGTQVTVSS |
| VHH3 | SEQ ID NO: 3 | DVQLQESGGGLVQPGESLKLSCLVSGVTYHFYD |
| | | ITWFRQAPGLEREGVSCISYASGTTKFADSVKG |
| | | RFTISRDNAKNAVYLEMNSLKPEDTAVYFCATQ |
| | | GSGSCSDEMSAGSVLSWGQGTQVTVSS |
| VHH4 | SEQ ID NO: 4 | DVQLQESGGGLVQPGESLRLSCLVSGVTYHFYD |
| | | ITWFRQAPGLEREGVSCISYASGSTKFADSVKG |
| | | RFTISRDNAKNAVYLQMNSLKPEDTAVYFCAMQ |
| | | GSGSCSDEMSADSVLSWGQGTQVTVSS |
| VHH5 | SEQ ID NO: 5 | DVQLQESGGGLVQPGESLRLSCLVSGVTYHFYD |
| | | ITWFRQAPGLEREGVSCISYASGSTKFADSVKG |
| | | RFTISRDNAKNAVYLQMNSLKPEDTAVYFCATQ |
| | | GSGSCSDEMSADSVLSWGQGTQVTVSS |
| VHH6 | SEQ ID NO: 6 | DVQLQESGGGLVQTGGSLRLSCAASGLPFSKYA |
| | | IAWFRQAPGKESEFLVVVSWSGDKFYQDNMKGR |
| | | CTISRDNAKNTAYLQINSLQPEDTAVYYCAASG |
| | | SYPVTDRYFYNFWGQGTQVTVSS |
| VHH7 | SEQ ID NO: 7 | DVQLQESGGGLAQPGGSLRLSCAASGFTFSSYD |
| | | MGWYRQVPGKECELVAAIFSAGGSTIYADSVKG |
| | | RFTISRDNAKNTVFLQMNSLKPEDTAVYYCNKA |
| | | PPFTTIQPTCVMIDTQLGAWGQGTQVTVSS |
| VHH8 | SEQ ID NO: 8 | DVQLQESGGGLAQPGGSLRLSCAASGFTFSSYD |
| | | MGWYRQAPGKECELVAAIFSADGSTIYADSVKG |
| | | RFTISSDNAKNTVFLQMNSLKPEDTAVYYCNKA |
| | | PPFTTIQPMCVMIDTQLGAWGQGTQVTVSS |
| VHH9 | SEQ ID NO: 9 | DVQLQESGGGLVQAGGSLRLSCAASGRGGASYA |
| | | MGWFRQAPGKEREFVAAISWTGDTTYNVDSVKG |
| | | RFTISSDNAKNPVYLDMSSLKPEDTAVYYCAAA |
| | | HWTRVVAKGAMADEKKYEYWGQGTQVTVSS |
| VHH10 | SEQ ID NO: 10 | DVQLQESGGGSVQAGGSLRLSCAASGRTFSSNF |
| | | MGWFRQAPNKDREFVAAIHWSGGSRAYADSVKG |
| | | RFTISRDNTKDTVYLLVNSLKLEDTAVYYCAAE |
| | | ISWQTTGWTNLGSWGQGTQVTVSS |
| VHH11 | SEQ ID NO: 11 | DVQLQESGGGLVQAGGSLRLSCAASGRTFSSDF |
| | | MGWFRQAPGKEREFVAAIHWSGGSTSYADSVKG |
| | | RFTISRDNAKDTVYLLMNSLKLEDTAVYYCAAE |
| | | IRWLTTGWANLGSWGQGTQVTVSS |
| VHH12 | SEQ ID NO: 12 | DVQLQESGGGLVQAGGSLRLSCAASGRTFSSDF |
| | | MGWFRQAPGKEREFVAAIHWSGGSRSYADSVKG |
| | | RFTISRDNAKDTVYLLMNSLKLEDTAVYYCAAE |
| | | IRWQTTGWANLGSWGQGTQVTVSS |
| VHH13 | SEQ ID NO: 13 | DVQLQESGGGLAQPGGPLRLSCAASGRTFTSDE |
| | | MGWFRQAPGKEREFVAAIHWSGGSRSYADSVKG |
| | | RFTISRDNAKDTVYLLMNSLKLEDTAVYYCAAE |
| | | IRWQTAGWVNLGSWGQGTQVTVSS |
| VHH14 | SEQ ID NO: 14 | DVQLQESGGGLVQPGGSLRLSCEASGVTFNFYS |
| | | MTWYRQAPGKERELVADITSEGRTEYAHSVKGR |
| | | FIISRDDFGTMVYLQMNSLKPEDTAVYYCYTGG |
| | | SWGQGTQVTVSS |
| VHH15 | SEQ ID NO: 15 | DVQLQESGGGLVQAGGSLRLSCAVSGSTFRLNV |
| | | MGWYRQAPGKLRDLVAGITADGGVTNYADSVKG |
| | | RFIISRDNAKNTVYLQMNSLKPEDTAIYYCNTA |
| | | PANWGSWGQGTQVTVSS |
| VHH16 | SEQ ID NO: 16 | DVQLQESGGGLVQPGGSLRLSCTASGSIFSSLT |
| | | MAWWRQAPGKQRELVAGITTDGSTDYADSVKGR |
| | | FTISRDNAKNTVYLQMDSLKPEDTAVYYCNKSP |
| | | YYDPTDYDSWGQGTQVTVSS |

[24] A compound comprising a VHH antibody domain or a fragment thereof, wherein
   (A) said VHH antibody domain consists of any one of the VHH sequences VHH1 to VHH16 shown in the Table of VHH Sequences;
   (B) said VHH antibody domain consists of a VHH sequence as defined in (A) with modification, wherein the modification is that said sequence is humanized;
   (C) said VHH antibody domain consists of a VHH sequence as defined in (A) with modification, wherein the modification is the replacement, addition or deletion of up to 25 amino acids; or
   (D) said VHH antibody domain consists of a VHH sequence that is at least 75% identical to a VHH sequence referred to in (A).
[25] The compound of any one of items or [24], wherein said fragment of said VHH antibody domain comprises at least 75% of the amino acids of the sequence of said VHH antibody domain.
[26] The compound of any one of items or [24], wherein said fragment of said VHH antibody domain comprises at least 80% of the amino acids of the sequence of said VHH antibody domain.
[27] The compound of any one of items or [24], wherein said fragment of said VHH antibody domain comprises at least 85% of the amino acids of the sequence of said VHH antibody domain.
[28] The compound of any one of items or [24], wherein said fragment of said VHH antibody domain comprises at least 90% of the amino acids of the sequence of said VHH antibody domain.
[29] The compound of any one of items or [24], wherein said fragment of said VHH antibody domain comprises at least 95% of the amino acids of the sequence of said VHH antibody domain.
[30] The compound of any one of items or [24], wherein said fragment of said VHH antibody domain comprises at least 98% of the amino acids of the sequence of said VHH antibody domain.
[31] The compound of any one of items or [24], wherein said fragment of said VHH antibody domain comprises at least 99% of the amino acids of the sequence of said VHH antibody domain.
[32] The compound of any one of items to [31], wherein said fragment of said VHH antibody domain comprises complementarity determining regions CDR1, CDR2 and CDR3.
[33] The compound of any one of items to [32], wherein said fragment of said VHH antibody domain comprises at least the sequence from the N-terminus of CDR1 to the C-terminus of CDR3 of said VHH antibody domain.
[24]0 The compound of any one of items to [33], wherein in (A) said fragment of said VHH antibody domain comprises all the complementarity determining regions (CDRs) of said VHH antibody domain.
[35] The compound of any one of items to [34], wherein in (B) said humanization of said sequence is by replacing at least one amino acid of said sequence by the corresponding amino acid of a human VH (variable heavy) domain.
[36] The compound of any one of items to [35], wherein in (B) said humanization of said sequence is by replacing up to 25 amino acids of said sequence by the corresponding amino acids of a human VH domain.
[37] The compound of any one of items to [35], wherein in (B) said humanization of said sequence is by replacing up to 20 amino acids of said sequence by the corresponding amino acids of a human VH domain.
[38] The compound of any one of items to [35], wherein in (B) said humanization of said sequence is by replacing up to 15 amino acids of said sequence by the corresponding amino acids of a human VH domain.
[39] The compound of any one of items to [35], wherein in (B) said humanization of said sequence is by replacing up to 10 amino acids of said sequence by the corresponding amino acids of a human VH domain.
[40] The compound of any one of items to [35], wherein in (B) said humanization of said sequence is by replacing up to 5 amino acids of said sequence by the corresponding amino acids of a human VH domain.
[41] The compound of any one of items to [35], wherein in (B) said humanization of said sequence is by replacing up to 3 amino acids of said sequence by the corresponding amino acids of a human VH domain.
[42] The compound of any one of items to [35], wherein in (B) said humanization of said sequence is by replacing up to 2 amino acids of said sequence by the corresponding amino acids of a human VH domain.
[43] The compound of any one of items to [35], wherein in (B) said humanization of said sequence is by replacing one amino acid of said sequence by the corresponding amino acid of a human VH domain.
[44] The compound of any one of items to [43], wherein in (B) said humanization is within the framework regions of said VHH antibody domain and/or within the CDRs of said VHH antibody domain.
[45] The compound of any one of items to [43], wherein in (B) said humanization is within the framework regions of said VHH antibody domain, but not within the CDRs of said VHH antibody domain.
[46] The compound of any one of items to [43], wherein in (B) said humanization is within the CDRs of said VHH antibody domain, but not within the framework regions of said VHH antibody domain.
[47] The compound of any one of items to or [46], wherein in (B) said humanization within the CDRs of said VHH antibody domain is within CDR1, CDR2 and/or CDR3.
[48] The compound of any one of items to or to [47], wherein in (B) said humanization within the CDRs of said VHH antibody domain is within CDR1 and/or CDR2.
[49] The compound of any one of items to or to [48], wherein in (B) said humanization within the CDRs of said VHH antibody domain is within CDR1.
[50] The compound of any one of items to or to [49], wherein in (B) said humanization within the CDRs of said VHH antibody domain is within CDR2.
[51] The compound of any one of items to [50], wherein in (B) said humanization within the CDRs of said VHH antibody domain is not within CDR3.
[52] The compound of any one of items to [51], wherein in (C) the modification is the replacement, addition or deletion of up to 20 amino acids.
[53] The compound of any one of items to [51], wherein in (C) the modification is the replacement, addition or deletion of up to 15 amino acids.
[54] The compound of any one of items to [51], wherein in (C) the modification is the replacement, addition or deletion of up to 10 amino acids.
[55] The compound of any one of items to [51], wherein in (C) the modification is the replacement, addition or deletion of up to 5 amino acids.
[56] The compound of any one of items to [51], wherein in (C) the modification is the replacement, addition or deletion of up to 3 amino acids.
[57] The compound of any one of items to [51], wherein in (C) the modification is the replacement, addition or deletion of up to 2 amino acids.
[58] The compound of any one of items to [51], wherein in (C) the modification is the replacement, addition or deletion of one amino acid.
[59] The compound of any one of items to [58], wherein the modification in (C) comprises only the replacement, but not the addition or deletion of amino acids.
[60] The compound of any one of items to [59], wherein in (A) said VHH antibody domain or fragment thereof comprises the complementarity determining regions CDR1, CDR2 and CDR3 of one of VHH1 to VHH16 as shown in the Table of CDRs.
[61] The compound of any one of items to [60], wherein in (B) to (D)
   (a) said VHH antibody domain or fragment thereof comprises the complementarity determining regions CDR1, CDR2 and CDR3 of one of VHH1 to VHH16 as shown in the Table of CDRs;
   (b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized;
   (c) said VHH antibody domain or fragment thereof comprises the complementarity determining regions CDR1, CDR2 and CDR3 as defined in (a) with modification, wherein the modification is
      the replacement, addition or deletion of up to three amino acids in CDR1,
      the replacement, addition or deletion of up to three amino acids in CDR2 and/or
      the replacement, addition or deletion of up to three amino acids in CDR3.
[62] The compound of item [61], wherein the modification in (b) is that the sequence of CDR1 and/or CDR2, but not the sequence of CDR3 is humanized.
[63] The compound of any one of items or [62], wherein the modification in (b) is that the sequence of CDR1 is humanized, but not the sequence of CDR2 and CDR3.
[64] The compound of any one of items or [62], wherein the modification in (b) is that the sequence of CDR2 is humanized, but not the sequence of CDR1 and CDR3.
[65] The compound of any one of items to [64], wherein the modification in (b) is that the sequence of one, but not more than one of CDR1, CDR2 and CDR3 is humanized.
[66] The compound of any one of items to [65], wherein said humanization of said CDR(s) is by replacing at least one amino acid in the sequence of said CDR by the corresponding amino acid of a human VH domain.
[67] The compound of any one of items to [66], wherein said humanization of said CDR(s) is by replacing up to three amino acids in the sequence of said CDR by the corresponding amino acid of a human VH domain.
[68] The compound of any one of items to [66], wherein said humanization of said CDR(s) is by replacing up to three amino acids in the sequence of CDR1 and/or CDR2 and up to one amino acid in the sequence of CDR3 by the corresponding amino acid of a human VH domain.
[69] The compound of any one of items to [66], wherein said humanization of said CDR(s) is by replacing up to two amino acids in the sequence of said CDR by the corresponding amino acid of a human VH domain.
[70] The compound of any one of items to [66], wherein said humanization of said CDR(s) is by replacing up to two amino acids in the sequence of CDR1 and/or CDR2 and up to one amino acid in the sequence of CDR3 by the corresponding amino acid of a human VH domain.
[71] The compound of any one of items to [70], wherein said humanization of said CDR(s) is by replacing one amino acid in the sequence of said CDR by the corresponding amino acid of a human VH domain.
[72] The compound of any one of items to [71], wherein the modification in (c) is
   the replacement, addition or deletion of up to three amino acids in CDR1,
   the replacement, addition or deletion of up to three amino acids in CDR2 and/or
   the replacement, addition or deletion of up to two amino acids in CDR3.
[73] The compound of any one of items to [71], wherein the modification in (c) is
   the replacement, addition or deletion of up to three amino acids in CDR1,
   the replacement, addition or deletion of up to three amino acids in CDR2 and/or
   the replacement, addition or deletion of up to one amino acid in CDR3.
[74] The compound of any one of items to [71], wherein the modification in (c) is
   the replacement, addition or deletion of up to three amino acids in CDR1, and/or
   the replacement, addition or deletion of up to three amino acids in CDR2,
wherein the sequence of CDR3 is unmodified.
[75] The compound of any one of items to [71], wherein the modification in (c) is
   the replacement, addition or deletion of up to two amino acids in CDR1,
   the replacement, addition or deletion of up to two amino acids in CDR2 and/or
   the replacement, addition or deletion of up to two amino acids in CDR3;
[76] The compound of any one of items to [71], wherein the modification in (c) is
   the replacement, addition or deletion of up to two amino acids in CDR1;
   the replacement, addition or deletion of up to two amino acids in CDR2; and/or
   the replacement, addition or deletion of up to one amino acid in CDR3.
[77] The compound of any one of items to [71], wherein the modification in (c) is
   the replacement, addition or deletion of up to two amino acids in CDR1 and/or
   the replacement, addition or deletion of up to two amino acids in CDR2;
wherein the sequence of CDR3 is unmodified.
[78] The compound of any one of items to [71], wherein the modification in (c) is
   the replacement, addition or deletion of up to two amino acids in CDR1,
wherein the sequence of CDR2 and CDR3 is unmodified.
[79] The compound of any one of items to [71], wherein the modification in (c) is
   the replacement, addition or deletion of up to two amino acids in CDR2,
wherein the sequence of CDR1 and CDR3 is unmodified.
[80] The compound of any one of items to [71], wherein the modification in (c) is
   the replacement, addition or deletion of up to one amino acid in CDR1;
   the replacement, addition or deletion of up to one amino acid in CDR2; and/or
   the replacement, addition or deletion of up to one amino acid in CDR3.
[81] The compound of any one of items to [71], wherein the modification in (c) is
   the replacement, addition or deletion of one amino acid in CDR1 and/or
   the replacement, addition or deletion of one amino acid in CDR2;
wherein the sequence of CDR3 is unmodified.
[82] The compound of any one of items to [71], wherein the modification in (c) is
   the replacement, addition or deletion of one amino acid in CDR1,
wherein the sequence of CDR2 and CDR3 is unmodified.
[83] The compound of any one of items to [71], wherein the modification in (c) is
   the replacement, addition or deletion of one amino acid in CDR2,
wherein the sequence of CDR1 and CDR3 is unmodified.
[84] The compound of any one of items to [83], wherein the modification in (c) comprises only the replacement, but not the addition or deletion of amino acids.
[85] The compound according to any one of items [1] to [84], wherein said compound comprises a VHH antibody domain (not only a fragment of a VHH antibody domain).
[86] The compound according to any one of items [1] to [84], wherein said compound consists of a fragment of a VHH antibody domain (not a full-length VHH antibody domain).
[87] The compound according to any one of items [1] to or [86], wherein said fragment consists of at least 100 amino acids.
[88] The compound according to any one of items [1] to or [86], wherein said fragment consists of at least 105 amino acids.
[89] The compound according to any one of items [1] to or [86], wherein said fragment consists of at least 110 amino acids.
[90] The compound according to any one of items [1] to or [86], wherein said fragment consists of at least 115 amino acids.
[91] The compound according to any one of items [1] to [90], wherein said compound is capable of specifically binding to NKp30.
[92] The compound according to any one of items [1] to [91], wherein said VHH antibody domain or fragment thereof is capable of specifically binding to NKp30.
[93] The compound according to any one of items [1] to [92], wherein said VHH antibody domain or fragment thereof binds to recombinant human NKp30 with a KD of 1×106 M or stronger.
[94] The compound according to any one of items [1] to [92], wherein said VHH antibody domain or fragment thereof binds to recombinant human NKp30 with a KD of 1×10⁻⁷ M or stronger.
[95] The compound according to any one of items [1] to [92], wherein said VHH antibody domain or fragment thereof binds to recombinant human NKp30 with a KD of 1×10⁻⁸ M or stronger.
[96] The compound according to any one of items [1] to [92], wherein said VHH antibody domain or fragment thereof binds to recombinant human NKp30 with a KD of 1×10⁻⁹ M or stronger.
[97] The compound according to any one of items [1] to [96], wherein said VHH antibody domain or fragment thereof binds to recombinant human NKp30 with a kₒₙ of 1×10⁴ (1/Ms) or higher.
[98] The compound according to any one of items [1] to [96], wherein said VHH antibody domain or fragment thereof binds to recombinant human NKp30 with a kₒₙ of 1×105 (1/Ms) or higher.
[99] The compound according to any one of items [1] to [98], wherein said VHH antibody domain or fragment thereof binds to recombinant human NKp30 with a k_{off} of 5×10⁻³ (1/s) or lower.
[100] The compound according to any one of items [1] to [98], wherein said VHH antibody domain or fragment thereof binds to recombinant human NKp30 with a k_{off} of 1×10⁻³ (1/s) or lower.
[101] The compound according to any one of items [1] to [98], wherein said VHH antibody domain or fragment thereof binds to recombinant human NKp30 with a k_{off} of 1×10⁻⁴ (1/s) or lower.
[102] The compound according to any one of items [1] to [98], wherein said VHH antibody domain or fragment thereof binds to recombinant human NKp30 with a k_{off} of 1×10⁻⁵ (1/s) or lower.
[103] The compound according to any one of items [1] to [98], wherein said VHH antibody domain or fragment thereof binds to recombinant human NKp30 with a k_{off} of 1×10⁻⁶ (1/s) or lower.
[104] The compound according to any one of items [1] to [98], wherein said VHH antibody domain or fragment thereof binds to recombinant human NKp30 with a k_{off} of 1×10⁻⁷ (1/s) or lower.
[105] The compound according to any one of items [1] to [104], wherein in (b) and (c) the VHH antibody domain or fragment thereof binds to recombinant human NKp30 with an affinity (KD value) that is by not more than a factor of 5 weaker than the binding of the corresponding VHH antibody domain without modification.
[106] The compound according to any one of items [1] to [104], wherein in (b) and (c) the VHH antibody domain or fragment thereof binds to recombinant human NKp30 with an affinity (KD value) that is by not more than a factor of 2 weaker than the binding of the corresponding VHH antibody domain without modification.
[107] The compound according to any one of items [1] to [104], wherein in (b) and (c) the VHH antibody domain or fragment thereof binds to recombinant human NKp30 with an affinity (KD value) that is by not more than a factor of 1.5 weaker than the binding of the corresponding VHH antibody domain without modification.
[108] The compound according to any one of items [1] to [107], wherein in (b) and (c) the VHH antibody domain or fragment thereof binds to recombinant human NKp30 with an affinity (KD value) that is by not more than a factor of 5 stronger than the binding of the corresponding VHH antibody domain without modification.
[109] The compound according to any one of items [1] to [107], wherein in (b) and (c) the VHH antibody domain or fragment thereof binds to recombinant human NKp30 with an affinity (KD value) that is by not more than a factor of 2 stronger than the binding of the corresponding VHH antibody domain without modification.
[110] The compound according to any one of items [1] to [107], wherein in (b) and (c) the VHH antibody domain or fragment thereof binds to recombinant human NKp30 with an affinity (KD value) that is by not more than a factor of 1.5 stronger than the binding of the corresponding VHH antibody domain without modification.
[111] The compound according to any one of items to [110], wherein in (B) and (C) the VHH antibody domain binds to recombinant human NKp30 with an affinity (KD value) that is by not more than a factor of 5 weaker than the binding of the corresponding VHH antibody domain without modification.
[112] The compound according to any one of items to [110], wherein in (B) and (C) the VHH antibody domain binds to recombinant human NKp30 with an affinity (KD value) that is by not more than a factor of 2 weaker than the binding of the corresponding VHH antibody domain without modification.
[113] The compound according to any one of items to [110], wherein in (B) and (C) the VHH antibody domain binds to recombinant human NKp30 with an affinity (KD value) that is by not more than a factor of 1.5 weaker than the binding of the corresponding VHH antibody domain without modification.
[114] The compound according to any one of items to [113], wherein in (B) and (C) the VHH antibody domain binds to recombinant human NKp30 with an affinity (KD value) that is by not more than a factor of 5 stronger than the binding of the corresponding VHH antibody domain without modification.
[115] The compound according to any one of items to [113], wherein in (B) and (C) the VHH antibody domain binds to recombinant human NKp30 with an affinity (KD value) that is by not more than a factor of 2 stronger than the binding of the corresponding VHH antibody domain without modification.
[116] The compound according to any one of items to [113], wherein in (B) and (C) the VHH antibody domain binds to recombinant human NKp30 with an affinity (KD value) that is by not more than a factor of 1.5 stronger than the binding of the corresponding VHH antibody domain without modification.
[117] The compound according to any one of items to [116], wherein in (D) the affinity (KD value) of the binding of the VHH antibody domain to human NKp30 is by not more than a factor of 5 weaker than the affinity (KD value) of the binding to human NKp30 of a VHH antibody domain consisting of the sequence from the Table of VHH Sequences that has the highest degree of sequence identity with the sequence of said VHH antibody domain of (D).
[118] The compound according to any one of items to [116], wherein in (D) the affinity (KD value) of the binding of the VHH antibody domain to human NKp30 is by not more than a factor of 2 weaker than the affinity (KD value) of the binding to human NKp30 of a VHH antibody domain consisting of the sequence from the Table of VHH Sequences that has the highest degree of sequence identity with the sequence of said VHH antibody domain of (D).
[119] The compound according to any one of items to [116], wherein in (D) the affinity (KD value) of the binding of the VHH antibody domain to human NKp30 is by not more than a factor of 1.5 weaker than the affinity (KD value) of the binding to human NKp30 of a VHH antibody domain consisting of the sequence from the Table of VHH Sequences that has the highest degree of sequence identity with the sequence of said VHH antibody domain of (D).
[120] The compound according to any one of items to [119], wherein in (D) the affinity (KD value) of the binding of the VHH antibody domain to human NKp30 is by not more than a factor of 5 stronger than the affinity (KD value) of the binding to human NKp30 of a VHH antibody domain consisting of the sequence from the Table of VHH Sequences that has the highest degree of sequence identity with the sequence of said VHH antibody domain of (D).
[121] The compound according to any one of items to [119], wherein in (D) the affinity (KD value) of the binding of the VHH antibody domain to human NKp30 is by not more than a factor of 2 stronger than the affinity (KD value) of the binding to human NKp30 of a VHH antibody domain consisting of the sequence from the Table of VHH Sequences that has the highest degree of sequence identity with the sequence of said VHH antibody domain of (D).
[122] The compound according to any one of items to [119], wherein in (D) the affinity (KD value) of the binding of the VHH antibody domain to human NKp30 is by not more than a factor of 1.5 stronger than the affinity (KD value) of the binding to human NKp30 of a VHH antibody domain consisting of the sequence from the Table of VHH Sequences that has the highest degree of sequence identity with the sequence of said VHH antibody domain of (D).
[123] The compound according to any one of items to [122], wherein said KD value/said Kon rate/said k_{off} rate is measured by kinetic measurements by biolayer interferometry at 25° C. and 1000 rpm in KB Buffer (PBS+0.1% Tween-20+1% BSA).
[124] The compound according to any one of items [1] to [123], wherein said VHH antibody domain or fragment thereof competes with human B7-H6 for binding to human NKp30.
[125] The compound according to any one of items [1] to [123], wherein said VHH antibody domain or fragment thereof does not compete with human B7-H6 for binding to human NKp30.
[126] The compound according to any one of items [1] to [123], wherein said VHH antibody domain or fragment thereof partially competes with human B7-H6 for binding to human NKp30.
[127] The compound of any one of items to [126], wherein said VHH antibody domain of (A) comprises any one of the VHH sequences VHH1, VHH2, VHH3, VHH4, VHH5, VHH8, VHH9, VHH10, VHH11, VHH12, VHH13, VHH14, VHH15 or VHH16 shown in the Table of VHH Sequences. (As can be seen e.g. from Example 3, Table 1, for compounds with such a VHH antibody domain a KD in the range of E-09 or stronger was observed for binding to NKp30.)
[128] The compound of any one of items to [126], wherein said VHH antibody domain of (A) comprises any one of the VHH sequences VHH1, VHH3, VHH5, VHH8, VHH10, VHH12, VHH13, VHH14, VHH15 or VHH16 shown in the Table of VHH Sequences. (As can be seen e.g. from Example 3, Table 1, for compounds with such a VHH antibody domain a KD in the range of E-10 or stronger was observed for binding to NKp30.)
[129] The compound of any one of items to [126], wherein said VHH antibody domain of (A) comprises the VHH sequence VHH15 shown in the Table of VHH Sequences. (As can be seen e.g. from Example 3, Table 1, for compounds with such a VHH antibody domain a KD in the range of E-12 or stronger and a k_{off} in the range of E-07 or lower was observed for binding to NKp30.)
[130] The compound of any one of items to [126], wherein said VHH antibody domain of (A) comprises any one of the VHH sequences VHH1, VHH2, VHH3, VHH5, VHH6, VHH8, VHH9, VHH10, VHH11, VHH12, VHH13, VHH14, VHH15 or VHH16 shown in the Table of VHH Sequences. (As can be seen e.g. from Example 3, Table 1, for compounds with such a VHH antibody domain a kₒₙ in the range of E-05 was observed for binding to NKp30.)
[131] The compound of any one of items to [126], wherein said VHH antibody domain of (A) comprises any one of the VHH sequences VHH1, VHH2, VHH3, VHH4, VHH5, VHH8, VHH10, VHH11, VHH12, VHH13, VHH14, VHH15 or VHH16 shown in the Table of VHH Sequences. (As can be seen e.g. from Example 3, Table 1, for compounds with such a VHH antibody domain a k_{off} in the range of E-04 or lower was observed for binding to NKp30.)
[132] The compound of any one of items to [126], wherein said VHH antibody domain of (A) comprises any one of the VHH sequences VHH10, VHH15 or VHH16 shown in the Table of VHH Sequences. (As can be seen e.g. from Example 3, Table 1, for compounds with such a VHH antibody domain a k_{off} in the range of E-05 or lower was observed for binding to NKp30.)
[133] The compound of any one of items to [126], wherein said VHH antibody domain of (A) comprises any one of the VHH sequences VHH1, VHH3, VHH5, VHH12, VHH13, VHH14, VHH15 or VHH16 shown in the Table of VHH Sequences. (As can be seen e.g. from Example 3, Table 1, for compounds with such a VHH antibody domain competition with B7-H6 is observed. As shown in Example 4, such VHH antibody domains result in compounds that mediate lysis of targeted cells with high efficiency.)
[134] The compound of any one of items to [126], wherein said VHH antibody domain of (A) comprises any one of the VHH sequences VHH1, VHH5 or VHH16 shown in the Table of VHH Sequences.
[135] The compound of any one of items to [126], wherein said VHH antibody domain of (A) comprises any one of the VHH sequences VHH2, VHH6, VHH7, VHH8, VHH9, VHH10 or VHH11 shown in the Table of VHH Sequences. (As can be seen e.g. from Example 3, Table 1, for compounds with such a VHH antibody domain no competition with B7-H6 is observed. As shown in Example 4, such VHH antibody domains result in compounds that mediate lysis of targeted cells, but with a lower efficiency.)
[136] The compound of any one of items to [126], wherein said VHH antibody domain of (A) comprises any one of the VHH sequences VHH2, VHH4 or VHH8 shown in the Table of VHH Sequences.
[137] The compound of any one of items to [126], wherein said VHH antibody domain of (A) comprises any one of the VHH sequences VHH2 or VHH8 shown in the Table of VHH Sequences.
[138] The compound of any one of items to [126], wherein said VHH antibody domain of (A) comprises the VHH sequence VHH4 shown in the Table of VHH Sequences. (As can be seen e.g. from Example 3, Table 1, for compounds with such a VHH antibody domain partial competition with B7-H6 is observed.)
[139] The compound of any one of items to [126], wherein said VHH antibody domain of (A) comprises any one of the VHH sequences VHH12 or VHH15 shown in the Table of VHH Sequences. (As can be seen e.g. from Example 3, for compounds with such a VHH antibody domain no competition with the partial competitor VHH4 is observed.)
[140] The compound of any one of items to [126], wherein said VHH antibody domain of (A) comprises any one of the VHH sequences VHH8, VHH9, VHH10 or VHH11 shown in the Table of VHH Sequences. (As can be seen e.g. from Example 3, compounds with such a VHH antibody domain belong to a specific epitope bin with common characteristics.)
[141] The compound of any one of items to [126], wherein said VHH antibody domain of (A) comprises any one of the VHH sequences VHH2 or VHH6 shown in the Table of VHH Sequences. (As can be seen e.g. from Example 3, compounds with such a VHH antibody domain belong to a specific epitope bin with common characteristics.)
[142] The compound of any one of items to [126], wherein said VHH antibody domain of (A) comprises any one of the VHH sequences VHH2, VHH6 or VHH7 shown in the Table of VHH Sequences. (As can be seen e.g. from Example 3, compounds with such a VHH antibody domain are unique with respect to their epitope targeting.)
[143] The compound of any one of items [1] to or to [142], wherein in (a) said VHH antibody domain or fragment thereof comprises the complementarity determining regions CDR1, CDR2 and CDR3 of any one of the VHH sequences VHH1, VHH2, VHH3, VHH4, VHH5, VHH8, VHH9, VHH10, VHH11, VHH12, VHH13, VHH14, VHH15 or VHH16 shown in the Table of CDRs. (As can be seen e.g. from Example 3, Table 1, for compounds with such a VHH antibody domain a KD in the range of E-09 or stronger was observed for binding to NKp30.)
[144] The compound of any one of items [1] to or to [142], wherein in (a) said VHH antibody domain or fragment thereof comprises the complementarity determining regions CDR1, CDR2 and CDR3 of one of any one of VHH1, VHH3, VHH5, VHH8, VHH10, VHH12, VHH13, VHH14, VHH15 or VHH16 shown in the Table of CDRs. (As can be seen e.g. from Example 3, Table 1, for compounds with such a VHH antibody domain a KD in the range of E-10 or stronger was observed for binding to NKp30.)
[145] The compound of any one of items [1] to or to [142], wherein in (a) said VHH antibody domain or fragment thereof comprises the complementarity determining regions CDR1, CDR2 and CDR3 of one of VHH15 shown in the Table of CDRs. (As can be seen e.g. from Example 3, Table 1, for compounds with such a VHH antibody domain a KD in the range of E-12 or stronger and a k_{off} in the range of E-07 or lower was observed for binding to NKp30.)
[146] The compound of any one of items [1] to or to [142], wherein in (a) said VHH antibody domain or fragment thereof comprises the complementarity determining regions CDR1, CDR2 and CDR3 of one of VHH1, VHH2, VHH3, VHH5, VHH6, VHH8, VHH9, VHH10, VHH11, VHH12, VHH13, VHH14, VHH15 or VHH16 shown in the Table of CDRs. (As can be seen e.g. from Example 3, Table 1, for compounds with such a VHH antibody domain a kₒₙ in the range of E-05 was observed for binding to NKp30.)
[147] The compound of any one of items [1] to or to [142], wherein in (a) said VHH antibody domain or fragment thereof comprises the complementarity determining regions CDR1, CDR2 and CDR3 of one of VHH1, VHH2, VHH3, VHH4, VHH5, VHH8, VHH10, VHH11, VHH12, VHH13, VHH14, VHH15 or VHH16 shown in the Table of CDRs. (As can be seen e.g. from Example 3, Table 1, for compounds with such a VHH antibody domain a k_{off} in the range of E-04 or lower was observed for binding to NKp30.)
[148] The compound of any one of items [1] to or to [142], wherein in (a) said VHH antibody domain or fragment thereof comprises the complementarity determining regions CDR1, CDR2 and CDR3 of one of VHH10, VHH15 or VHH16 shown in the Table of CDRs. (As can be seen e.g. from Example 3, Table 1, for compounds with such a VHH antibody domain a k_{off} in the range of E-05 or lower was observed for binding to NKp30.)
[149] The compound of any one of items [1] to or to [142], wherein in (a) said VHH antibody domain or fragment thereof comprises the complementarity determining regions CDR1, CDR2 and CDR3 of one of VHH1, VHH3, VHH5, VHH12, VHH13, VHH14, VHH15 or VHH16 shown in the Table of CDRs. (As can be seen e.g. from Example 3, Table 1, for compounds with such a VHH antibody domain competition with B7-H6 is observed. As shown in Example 4, such VHH antibody domains result in compounds that mediate lysis of targeted cells with high efficiency.)
[150] The compound of any one of items [1] to or to [142], wherein in (a) said VHH antibody domain or fragment thereof comprises the complementarity determining regions CDR1, CDR2 and CDR3 of one of VHH1, VHH5 or VHH16 shown in the Table of CDRs.
[151] The compound of any one of items [1] to or to [142], wherein in (a) said VHH antibody domain or fragment thereof comprises the complementarity determining regions CDR1, CDR2 and CDR3 of one of VHH2, VHH6, VHH7, VHH8, VHH9, VHH10 or VHH11 shown in the Table of CDRs. (As can be seen e.g. from Example 3, Table 1, for compounds with such a VHH antibody domain no competition with B7-H6 is observed. As shown in Example 4, such VHH antibody domains result in compounds that mediate lysis of targeted cells, but with a lower efficiency.)
[152] The compound of any one of items [1] to or to [142], wherein in (a) said VHH antibody domain or fragment thereof comprises the complementarity determining regions CDR1, CDR2 and CDR3 of one of VHH2, VHH4 or VHH8 shown in the Table of CDRs.
[153] The compound of any one of items [1] to or to [142], wherein in (a) said VHH antibody domain or fragment thereof comprises the complementarity determining regions CDR1, CDR2 and CDR3 of one of VHH2 or VHH8 shown in the Table of CDRs.
[154] The compound of any one of items [1] to or to [142], wherein in (a) said VHH antibody domain or fragment thereof comprises the complementarity determining regions CDR1, CDR2 and CDR3 of VHH4 shown in the Table of CDRs. (As can be seen e.g. from Example 3, Table 1, for compounds with such a VHH antibody domain partial competition with B7-H6 is observed.)
[155] The compound of any one of items [1] to or to [142], wherein in (a) said VHH antibody domain or fragment thereof comprises the complementarity determining regions CDR1, CDR2 and CDR3 of one of VHH12 or VHH15 shown in the Table of CDRs. (As can be seen e.g. from Example 3, for compounds with such a VHH antibody domain no competition with the partial competitor VHH4 is observed.)
[156] The compound of any one of items [1] to or to [142], wherein in (a) said VHH antibody domain or fragment thereof comprises the complementarity determining regions CDR1, CDR2 and CDR3 of one of VHH8, VHH9, VHH10 or VHH11 shown in the Table of CDRs. (As can be seen e.g. from Example 3, compounds with such a VHH antibody domain belong to a specific epitope bin with common characteristics.)
[157] The compound of any one of items [1] to or to [142], wherein in (a) said VHH antibody domain or fragment thereof comprises the complementarity determining regions CDR1, CDR2 and CDR3 of one of VHH2 or VHH6 shown in the Table of CDRs. (As can be seen e.g. from Example 3, compounds with such a VHH antibody domain belong to a specific epitope bin with common characteristics.)
[158] The compound of any one of items [1] to or to [142], wherein in (a) said VHH antibody domain or fragment thereof comprises the complementarity determining regions CDR1, CDR2 and CDR3 of one of VHH2, VHH6 or VHH7 shown in the Table of CDRs. (As can be seen e.g. from Example 3, compounds with such a VHH antibody domain are unique with respect to their epitope targeting.)
[159] The compound according to any one of items [1] to [158], wherein said compound is a molecule.
[160] The compound according to any one of items [1] to [159], wherein said compound comprises or is a protein.
[160] The compound according to any one of items [1] to [160], wherein said compound is a protein.
[161] The compound according to any one of items to [161], wherein said compound comprises a VHH antibody domain or fragment thereof according to (A), (B) or (C).
[162] The compound according to any one of items to [161], wherein said compound comprises a VHH antibody domain or fragment thereof according to (A), (B) or (D).
[163] The compound according to any one of items to [161], wherein said compound comprises a VHH antibody domain or fragment thereof according to (A), (C) or (D).
[164] The compound according to any one of items to [161], wherein said compound comprises a VHH antibody domain or fragment thereof according to (A) or (B).
[165] The compound according to any one of items to [161], wherein said compound comprises a VHH antibody domain or fragment thereof according to (A) or (C).
[166] The compound according to any one of items to [161], wherein said compound comprises a VHH antibody domain or fragment thereof according to (A) or (D).
[167] The compound according to any one of items to [161], wherein said compound comprises a VHH antibody domain or fragment thereof according to (A).
[168] The compound according to any one of items to [161], wherein said compound comprises a VHH antibody domain or fragment thereof according to (B).
[170] The compound according to any one of items [1] to or to [169], wherein said VHH antibody domain or fragment thereof comprises complementarity determining regions according to (a) or (b).
[170] The compound according to any one of items [1] to or to [169], wherein said VHH antibody domain or fragment thereof comprises complementarity determining regions according to (a) or (c).
[171] The compound according to any one of items [1] to or to [169], wherein said VHH antibody domain or fragment thereof comprises complementarity determining regions according to (a).
[172] The compound according to any one of items [1] to or to [169], wherein said VHH antibody domain or fragment thereof comprises complementarity determining regions according to (b).
[173] The compound according to any one of items [1] to [173], wherein said compound further comprises a targeting moiety.
[174] The compound according to any one of items [1] to [174], wherein all components of said compound are covalently linked.
[175] The compound according to any one of items or [175], wherein said targeting moiety is a molecular group that specifically binds to a target molecule or fragment thereof.
[176] The compound according to item [176], wherein said target molecule is a receptor at the surface of a cell.
[177] The compound according to any one of items or [177], wherein said target molecule is an antigen that is present on the surface of a target cell.
[179] The compound according to any one of items to [178], wherein said targeting moiety is a protein, a peptide, a peptide mimetic, a nucleic acid, an oligonucleotide or a small molecule.
[180] The compound according to any one of items to [179], wherein said targeting moiety comprises or is a protein.
[181] The compound according to any one of items to [180], wherein said targeting moiety comprises a protein.
[182] The compound according to any one of items to [181], wherein said targeting moiety is a protein.
[183] The compound according to any one of items to [182], wherein said targeting moiety is a protein ligand that specifically binds to a receptor at the surface of a cell.
[184] The compound according to any one of items to [182], wherein said targeting moiety is an antibody or an antigen-binding fragment thereof.
[185] The compound according to any one of items to [184], wherein said targeting moiety is capable of specifically binding to a tumor-associated antigen.
[186] The compound according to any one of items [1] to [185], wherein said compound is a bispecific antibody.
[187] The compound according to any one of items [1] to [186], wherein said compound is a bispecific antibody prepared by the SEED (strand-exchange engineered domain) technology.
[188] The compound according to any one of items or [187], wherein one binding site of said bispecific antibody is formed by said VHH antibody domain or fragment thereof and one binding site of said bispecific antibody is formed by said targeting moiety.
[189] The compound according to any one of items to [188], wherein said antigen-binding fragment is selected from the group consisting of a Fab, a Fab′, a (Fab′)2, a Fv, a scFv, a diabody and a VHH.
[190] The compound according to any one of items to [188], wherein said antigen-binding fragment is selected from the group consisting of a Fab, a Fab′, a (Fab′)2 and a Fv.
[191] The compound according to any one of items to [188], wherein said antigen-binding fragment is a Fab.
[192] The compound according to any one of items to [188], wherein said antigen-binding fragment is selected from the group consisting of a scFv, a diabody and a VHH.
[193] The compound according to any one of items to [192], wherein said antigen-binding fragment is an antigen-binding fragment of an antibody with the SEED (strand-exchange engineered domain) format.
[194] The compound according to any one of items to [193], wherein said targeting moiety is capable of specifically binding to an antigen that is present on the surface of a target cell.
[195] The compound according to any one of items to [194], wherein said antibody is an antibody against an antigen that is present on the surface of a target cell.
[196] The compound according to any one of items to [195], wherein said antigen-binding fragment is an antigen-binding fragment of an antibody against an antigen that is present on the surface of a target cell.
[197] The compound according to any one of items to [196], wherein said binding of said targeting moiety to said antigen that is present on the surface of said target cell allows to recruit the compound specifically to said target cell.
[198] The compound according to any one of items to [197], wherein said antigen that is present on the surface of said target cell is a tumor-associated antigen.
[199] The compound according to any one of items to [198], wherein said targeting moiety is capable of specifically binding to a tumor-associated antigen.
[200] The compound according to any one of items to [199], wherein said tumor-associated antigen is an antigen that is present on the surface of a tumor cell.
[201] The compound according to any one of items to [199], wherein said tumor-associated antigen is EGFR (epidermal growth factor receptor).
[202] The compound according to any one of items [1] to [201], wherein said compound is a bispecific or multispecific molecule.
[203] The compound according to any one of items [1] to [202], wherein said compound comprises an antibody Fc region.
[204] The compound according to any one of items [1] to [203], wherein said compound comprises an antibody Fc region competent in Fc receptor binding.
[205] The compound according to any one of items [1] to [204], wherein said compound comprises an antibody Fc region that is not competent in Fc receptor binding.
[206] The compound according to any one of items [1] to [205], wherein said compound does not comprise an effector-competent antibody Fc region.
[207] The compound according to any one of items [1] to [205], wherein said compound comprises an effector-competent antibody Fc region.
[208] The compound according to any one of items [1] to [207], wherein said compound does not comprise an antibody Fc region capable of inducing ADCC (antibody-dependent cellular cytotoxicity).
[209] The compound according to any one of items [1] to [207], wherein said compound comprises an antibody Fc region capable of inducing ADCC.
[210] The compound according to any one of items [1] to [209], wherein said compound comprises an antibody Fc region that is not capable of inducing ADCC.
[211] The compound according to any one of items [1] to [210], wherein said compound is capable of inducing both FcγRIIIa signalling and positive (i.e. NK cell activating) NKp30 signalling.
[212] The compound according to any one of items [1] to [211], wherein said compound is capable of specifically binding to NKp30 on NK cells.
[213] The compound according to any one of items [1] to [212], wherein said compound is capable of activating NK cells by binding to NKp30 on said NK cells.
[214] The compound according to any one of items to [213], wherein binding of said compound to NKp30 on NK cells activates said NK cells.
[215] The compound according to any one of items [1] to [214], wherein said compound is an agonist of NKp30.
[216] The compound according to any one of items [1] to [215], wherein said compound is, upon binding of said VHH antibody domain or fragment thereof to NKp30 on NK cells, capable of activating NK cells.
[217] The compound according to any one of items [1] to [216], wherein binding of said compound to NKp30 on NK cells activates said NK cells.
[218] The compound according to any one of items to [217], wherein activation of NK cells is measured by measuring expression of the activation marker CD69 by flow cytometry.
[219] The compound according to any one of items [1] to [218], wherein said compound shows cytotoxic activity in a ⁵¹Cr release assay.
[220] The compound according to item [219], wherein said ⁵¹Cr release assay is carried out as a 4 h ⁵¹Cr release assay, wherein human PBMCs are used as effector cells at effector-to-target cell (E:T) ratios of 80:1, and wherein a higher percent lysis indicates improved cytotoxic activity.
[221] The compound according to any one of items to [220], wherein said ⁵¹Cr release assay is carried out as described in Repp et al., 2011.
[222] The compound according to any one of items [1] to [221], wherein binding of said compound to NKp30 on NK cells results in the release of interferon-γ (IFN-γ).
[223] The compound according to item [222], wherein said release of IFN-γ is measured as follows: isolated human NK cells are incubated overnight in medium containing 100 U/ml recombinant human interleukin-2, in different wells A431 cells are seeded and incubated for 3 h, the compound is added to a final concentration of 85 nM followed by addition of NK cells at an E:T ratio of 5:1, human IFN-γ is analyzed in the culture supernatant after 24 h by ELISA.
[224] The compound according to any one of items [1] to [223], wherein binding of said compound to NKp30 on NK cells results in the release of tumor necrosis factor-α (TNF-α).
[225] The compound according to item [224], wherein said release of TNF-α is measured as follows: isolated human NK cells are incubated overnight in medium containing 100 U/ml recombinant human interleukin-2, in different wells A431 cells are seeded and incubated for 3 h, the compound is added to a final concentration of 85 nM followed by addition of NK cells at an E:T ratio of 5:1, human TNF-α is analyzed in the culture supernatant after 24 h by ELISA.
[226] A pharmaceutical composition comprising the compound according to any one of items [1] to [225].
[227] The pharmaceutical composition according to item [226], wherein said pharmaceutical composition comprises a pharmaceutically acceptable carrier, diluent and/or excipient.
[228] The pharmaceutical composition according to any one of items to [227], wherein said pharmaceutical composition further includes at least one additional adjuvant, antioxidant, buffering agent, bulking agent, colorant, emulsifier, filler, flavoring agent, preservative, stabilizer, suspending agent and/or other customary pharmaceutical auxiliary.
[229] A compound according to any one of items [1] to or the pharmaceutical composition according to any one of items to for use as a medicament.
[230] A compound according to any one of items [1] to or the pharmaceutical composition according to any one of items to for use in the treatment of cancer.
[231] A compound according to any one of items [1] to or the pharmaceutical composition according to any one of items to for use in the treatment of a malignant tumor.
[232] A compound according to any one of items [1] to or the pharmaceutical composition according to any one of items to [228], wherein said compound/said pharmaceutical composition is for use in the treatment of a human.
[233] A method for treating a disease in a patient in need thereof, comprising the step of administering to said patient a therapeutically effective amount of the compound according to any one of items [1] to or the pharmaceutical composition according to any one of items to [228].
[234] The method according to item [233], wherein said disease is cancer.
[235] The method according to item [233], wherein said disease is a malignant tumor.
[236] The method according to any one of items [233] to [235], wherein said patient is a human.
[237] Use of the compound according to any one of items [1] to [225] or the pharmaceutical composition according to any one of items [226] to [228] for the manufacture of a medicament.
[238] Use of the compound according to any one of items [1] to [225] or the pharmaceutical composition according to any one of items [226] to [228] for the manufacture of a medicament for the treatment of cancer.
[239] Use of the compound according to any one of items [1] to [225] or the pharmaceutical composition according to any one of items [226] to [228] for the manufacture of a medicament for the treatment of a malignant tumor.
[240] The use according to any one of items [237] to [239], wherein said medicament is prepared for administration to a human.
[241] The compound or the pharmaceutical composition for use according to any one of items [230] or [231] or the method according to any one of items [234] or [235] or the use according to any one of items [238] or [239], wherein said cancer or malignant tumor is a human disease.
[242] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 1 (or a fragment thereof), with one or more amino acid substitutions compared to the sequence of SEQ ID NO: 1, wherein, if such amino acid substitutions are located outside of the CDRs, said amino acid substitution(s) are selected from the following: D1E, Q5V, L11V, T24A, E44G, H45L, G47W, F70I, V79L, K87R, P88A, Q116L.
[243] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 1 (or a fragment thereof), with at least the following amino acid substitutions: D1E, Q5V, L11V, V79L, K87R, P88A, Q116L; and with optionally one or more further amino acid substitutions compared to the sequence of SEQ ID NO: 1, wherein, if such further amino acid substitutions are located outside of the CDRs, such further amino acid substitution(s) are selected from the following: T24A, E44G, H45L, F70I.
[244] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 1 (or a fragment thereof), with the additional modification of (b) according to any one of items [1] to [11] or with the additional modification of (c) according to any one of items [1] or [12] to [22].
[245] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 1 (or a fragment thereof), with the additional modification of (b) according to any one of items [1] to [11] or with the additional modification of (c) according to any one of items [1] or [12] to [22]; and with one or more further amino acid substitutions compared to the sequence of SEQ ID NO: 1, wherein said amino acid substitution(s) are selected from the following: D1E, Q5V, L11V, T24A, E44G, H45L, G47W, F70I, V79L, K87R, P88A, Q116L.
[246] The compound of any one of items [1] to [22] or [85] to [110] or [123] to [126] or [143] to [161] or [170] to [225] or [232] or [245] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 1 (or a fragment thereof), with the additional modification of (b) according to any one of items [1] to [11] or with the additional modification of (c) according to any one of items [1] or [12] to [22]; and the amino acid substitutions D1E, Q5V, L11V, V79L, K87R, P88A, Q116L; and with optionally one or more further amino acid substitutions compared to the sequence of SEQ ID NO: 1, wherein said further amino acid substitution(s) are selected from the following: T24A, E44G, H45L, F70I.
[247] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or a fragment thereof.
[248] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound has a sequence represented by any one of the following SEQ ID NOs: SEQ ID NO: 67, SEQ ID NO: 68, SEQ ID NO: 69, or a fragment thereof.
[249] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound has a sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 77, or a fragment thereof.
[250] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound has a sequence represented by any one of the following SEQ ID NOs: SEQ ID NO: 67, SEQ ID NO: 68, SEQ ID NO: 69, SEQ ID NO: 77, or a fragment thereof.
[251] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 78, or a fragment thereof.
[252] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 67, SEQ ID NO: 68, SEQ ID NO: 69, SEQ ID NO: 78, or a fragment thereof.
[253] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence defined by SEQ ID NO: 77, or a fragment thereof.
[254] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence defined by SEQ ID NO: 78, or a fragment thereof.
[255] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or a fragment thereof, with the additional modification of (b) according to any one of items [1] to [11] or with the additional modification of (c) according to any one of items [1] or [12] to [22].
[256] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 67, SEQ ID NO: 68, SEQ ID NO: 69, or a fragment thereof, with the additional modification of (b) according to any one of items [1] to [11] or with the additional modification of (c) according to any one of items [1] or [12] to [22].
[257] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 77, or a fragment thereof, with the additional modification of (b) according to any one of items [1] to [11] or with the additional modification of (c) according to any one of items [1] or [12] to [22].
[258] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 67, SEQ ID NO: 68, SEQ ID NO: 69, SEQ ID NO: 77, or a fragment thereof, with the additional modification of (b) according to any one of items [1] to [11] or with the additional modification of (c) according to any one of items [1] or [12] to [22].
[259] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 78, or a fragment thereof, with the additional modification of (b) according to any one of items [1] to [11] or with the additional modification of (c) according to any one of items [1] or [12] to [22].
[260] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 67, SEQ ID NO: 68, SEQ ID NO: 69, SEQ ID NO: 78, or a fragment thereof, with the additional modification of (b) according to any one of items [1] to [11] or with the additional modification of (c) according to any one of items [1] or [12] to [22].
[261] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence defined by SEQ ID NO: 77 or a fragment thereof, with the additional modification of (b) according to any one of items [1] to [11] or with the additional modification of (c) according to any one of items [1] or [12] to [22].
[262] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence defined by SEQ ID NO: 78 or a fragment thereof, with the additional modification of (b) according to any one of items [1] to [11] or with the additional modification of (c) according to any one of items [1] or [12] to [22].
[263] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 2 (or a fragment thereof), with one or more amino acid substitutions compared to the sequence of SEQ ID NO: 2, wherein, if such amino acid substitutions are located outside of the CDRs, said amino acid substitution(s) are selected from the following: D1E, Q5V, L11V, A14P, V24A, F37V, A44G, R45L, S47W, V79L, K87R, P88A, Q118L.
[264] The compound of any one of items [1] to [22] or [85] to [110] or [123] to [126] or [143] to [161] or [170] to [225] or [232] or [263] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 2 (or a fragment thereof), with the amino acid substitutions D1E, Q5V, L11V, K87R, P88A, Q118L; and with optionally one or more further amino acid substitutions compared to the sequence of SEQ ID NO: 2, wherein, if such further amino acid substitutions are located outside of the CDRs, such further amino acid substitution(s) are selected from the following: A14P, V24A, A44G, R45L, S47W, V79L.
[265] The compound of any one of items [1] to [22] or [85] to [110] or [123] to [126] or [143] to [161] or [170] to [225] or [232] or [263] to [264] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 2 (or a fragment thereof), with the amino acid substitutions D1E, Q5V, L11V, A14P, V24A, K87R, P88A, Q118L; and with optionally one or more further amino acid substitutions compared to the sequence of SEQ ID NO: 2, wherein, if such further amino acid substitutions are located outside of the CDRs, such further amino acid substitution(s) are selected from the following: A44G, R45L, S47W, V79L.
[266] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 2 (or a fragment thereof), with the additional modification of (b) according to any one of items [1] to [11] or with the additional modification of (c) according to any one of items [1] or [12] to [22].
[267] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 2 (or a fragment thereof), with the additional modification of (b) according to any one of items [1] to [11] or with the additional modification of (c) according to any one of items [1] or [12] to [22]; and with one or more further amino acid substitutions compared to the sequence of SEQ ID NO: 2, wherein said amino acid substitution(s) are selected from the following: D1E, Q5V, L11V, A14P, V24A, F37V, A44G, R45L, S47W, V79L, K87R, P88A, Q118L.
[268] The compound of any one of items [1] to [22] or [85] to [110] or [123] to [126] or [143] to [161] or [170] to [225] or [232] or [267] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 2 (or a fragment thereof), with the additional modification of (b) according to any one of items [1] to [11] or with the additional modification of (c) according to any one of items [1] or [12] to [22]; and with the amino acid substitutions D1E, Q5V, L11V, K87R, P88A, Q118L; and with optionally one or more further amino acid substitutions compared to the sequence of SEQ ID NO: 2, wherein said further amino acid substitution(s) are selected from the following: A14P, V24A, A44G, R45L, S47W, V79L.
[269] The compound of any one of items [1] to [22] or [85] to [110] or [123] to [126] or [143] to [161] or [170] to [225] or [232] or [267] to [268] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by SEQ ID NO: 2 (or a fragment thereof), with the additional modification of (b) according to any one of items [1] to [11] or with the additional modification of (c) according to any one of items [1] or [12] to [22]; and with the amino acid substitutions D1E, Q5V, L11V, A14P, V24A, K87R, P88A, Q118L; and with optionally one or more further amino acid substitutions compared to the sequence of SEQ ID NO: 2, wherein said further amino acid substitution(s) are selected from the following: A44G, R45L, S47W, V79L.
[270] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 79, SEQ ID NO: 80, SEQ ID NO: 81, SEQ ID NO: 83, SEQ ID NO: 84, SEQ ID NO: 85, SEQ ID NO: 86, SEQ ID NO: 89, SEQ ID NO: 90, SEQ ID NO: 93, SEQ ID NO: 94, or a fragment thereof.
[271] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 79, SEQ ID NO: 80, SEQ ID NO: 83, SEQ ID NO: 86, SEQ ID NO: 89, SEQ ID NO: 90, SEQ ID NO: 94, or a fragment thereof.
[272] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ ID NO: 79, SEQ ID NO: 80, SEQ ID NO: 81, SEQ ID NO: 83, SEQ ID NO: 84, SEQ ID NO: 85, SEQ ID NO: 86, SEQ ID NO: 89, SEQ ID NO: 90, SEQ ID NO: 93, SEQ ID NO: 94, or a fragment thereof, with the additional modification of (b) according to any one of items [1] to [11] or with the additional modification of (c) according to any one of items [1] or [12] to [22].
[273] The compound of any one of items [1] to [22] or [85] to [110] or [123] to [126] or [143] to [161] or [170] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said compound comprises the sequence represented by any one of the following SEQ ID NOs: SEQ-ID SEQ ID NO: 79, SEQ ID NO: 80, SEQ ID NO: 83, SEQ ID NO: 86, SEQ ID NO: 89, SEQ ID NO: 90, SEQ ID NO: 94, or a fragment thereof, with the additional modification of (b) according to any one of items [1] to [11] or with the additional modification of (c) according to any one of items [1] or [12] to [22].
[274] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises the VHH sequence VHH1 shown in the Table of VHH Sequences or a fragment thereof, with additional modification of (B) or (C), wherein said modification of (B) or (C) consists of the substitution of one or more amino acids compared to the sequence of SEQ ID NO: 1, wherein said amino acid substitution(s) are selected from the following: D1E, Q5V, L11V, T24A, E44G, H45L, G47W, F70I, V79L, K87R, P88A, Q116L.
[275] The compound of any one of items [23] to [45] or [52] to [142] or [159] to [225] or [232] or [274] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises the VHH sequence VHH1 shown in the Table of VHH Sequences or a fragment thereof, with additional modification of (B) or (C), wherein said modification of (B) or (C) consists of the amino acid substitutions D1E, Q5V, L11V, V79L, K87R, P88A, Q116L compared to the sequence of SEQ ID NO: 1; and optionally one or more further amino acid substitutions compared to the sequence of SEQ ID NO: 1, wherein said further amino acid substitution(s) are selected from the following: T24A, E44G, H45L, F70I.
[276] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises a VHH sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or a fragment thereof.
[277] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises a VHH sequence represented by any one of the following SEQ ID NOs: SEQ ID NO: 67, SEQ ID NO: 68, SEQ ID NO: 69, or a fragment thereof.
[278] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises a VHH sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 77, or a fragment thereof.
[279] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises a VHH sequence represented by any one of the following SEQ ID NOs: SEQ ID NO: 67, SEQ ID NO: 68, SEQ ID NO: 69, SEQ ID NO: 77, or a fragment thereof.
[280] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises a VHH sequence represented by any one of the following SEQ ID NOs: SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 78, or a fragment thereof.
[281] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises a VHH sequence represented by any one of the following SEQ ID NOs: SEQ ID NO: 67, SEQ ID NO: 68, SEQ ID NO: 69, SEQ ID NO: 78, or a fragment thereof.
[282] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises the VHH sequence VHH1 shown in the Table of VHH Sequences or a fragment thereof, with additional modification of (B) or (C), wherein said modification of (B) or (C) consists of the substitution of one or more amino acids compared to the sequence of SEQ ID NO: 2, wherein said amino acid substitution(s) are selected from the following: D1E, Q5V, L11V, A14P, V24A, F37V, A44G, R45L, S47W, V79L, K87R, P88A, Q118L.
[283] The compound of any one of items [23] to [45] or [52] to [142] or [159] to [225] or [232] or [282] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises the VHH sequence VHH1 shown in the Table of VHH Sequences or a fragment thereof, with additional modification of (B) or (C), wherein said modification of (B) or (C) consists of the amino acid substitutions D1E, Q5V, L11V, K87R, P88A, Q118L compared to the sequence of SEQ ID NO: 2; and optionally one or more further amino acid substitutions compared to the sequence of SEQ ID NO: 2, wherein said further amino acid substitution(s) are selected from the following: A14P, V24A, A44G, R45L, S47W, V79L.
[284] The compound of any one of items [23] to [45] or [52] to [142] or [159] to [225] or [232] or [282] to [283] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises the VHH sequence VHH1 shown in the Table of VHH Sequences or a fragment thereof, with additional modification of (B) or (C), wherein said modification of (B) or (C) consists of the amino acid substitutions D1E, Q5V, L11V, A14P, V24A, K87R, P88A, Q118L compared to the sequence of SEQ ID NO: 2; and optionally one or more further amino acid substitutions compared to the sequence of SEQ ID NO: 2, wherein said further amino acid substitution(s) are selected from the following: A44G, R45L, S47W, V79L.
[285] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises a VHH sequence represented by any one of the following SEQ ID NOs: SEQ ID NO: 79, SEQ ID NO: 80, SEQ ID NO: 81, SEQ ID NO: 83, SEQ ID NO: 84, SEQ ID NO: 85, SEQ ID NO: 86, SEQ ID NO: 89, SEQ ID NO: 90, SEQ ID NO: 93, SEQ ID NO: 94, or a fragment thereof.
[286] The compound of any one of items [23] to [45] or [52] to [142] or [159] to [225] or [232] or the pharmaceutical composition for use according to any one of items [226] to [228] or [232] or the compound or pharmaceutical composition for use according to any one of items [229] to [231] or [241] or the method according to any one of items [233] to [236] or [241] or the use according to any one of items [237] to [241], wherein said VHH antibody domain comprises a VHH sequence represented by any one of the following SEQ ID NOs: SEQ ID NO: 79, SEQ ID NO: 80, SEQ ID NO: 83, SEQ ID NO: 86, SEQ ID NO: 89, SEQ ID NO: 90, SEQ ID NO: 94, or a fragment thereof.
[287] The compound of any one of items [1] to [225] or [232] or [242] to [286] or the pharmaceutical composition for use according to any one of items [226] to [228] or [232] or [242] to [286] or the compound or pharmaceutical composition for use according to any one of items [229] to [231] or [241] to [286] or the method according to any one of items [233] to [236] or [241] to [286] or the use according to any one of items [237] to [286], wherein said compound binds to NKp30 with an affinity of at least about 1×10⁶ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).
[288] The compound of any one of items [1] to [225] or [232] or [242] to [286] or the pharmaceutical composition for use according to any one of items [226] to [228] or [232] or [242] to [286] or the compound or pharmaceutical composition for use according to any one of items [229] to [231] or [241] to [286] or the method according to any one of items [233] to [236] or [241] to [286] or the use according to any one of items [237] to [286], wherein said compound binds to NKp30 with an affinity of at least about 5×10⁶ M⁻¹ in a binding assay by BLI (biolayer interferometry) with recombinant extracellular domain of NKp30 (NKp30-ECD, SEQ ID NO: 66) of NKp30 in KB buffer (PBS+0.1% Tween-20+1% BSA).
[289] The compound of any one of items [1] to [225] or [232] or [242] to [286] or the pharmaceutical composition for use according to any one of items [226] to [228] or [232] or [242] to [286] or the compound or pharmaceutical composition for use according to any one of items [229] to [231] or [241] to [286] or the method according to any one of items [233] to [236] or [241] to [286] or the use according to any one of items [237] to [286], wherein said compound binds to NKp30 with an affinity of at least about 1×10⁷ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).
[290] The compound of any one of items [1] to [225] or [232] or [242] to [286] or the pharmaceutical composition for use according to any one of items [226] to [228] or [232] or [242] to [286] or the compound or pharmaceutical composition for use according to any one of items [229] to [231] or [241] to [286] or the method according to any one of items [233] to [236] or [241] to [286] or the use according to any one of items [237] to [286], wherein said compound binds to NKp30 with an affinity of at least about 5×10⁷ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).
[291] The compound of any one of items [1] to [225] or [232] or [242] to [286] or the pharmaceutical composition for use according to any one of items [226] to [228] or [232] or [242] to [286] or the compound or pharmaceutical composition for use according to any one of items [229] to [231] or [241] to [286] or the method according to any one of items [233] to [236] or [241] to [286] or the use according to any one of items [237] to [286], wherein said compound binds to NKp30 with an affinity of at least about 1×10⁸ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).

### EXAMPLES

The following examples describe the preparation and characterization of VHH-based NKp30 binders as disclosed in the present disclosure, as well as related compounds and methods, along with comparative disclosure. It is understood that various embodiments of the disclosure reflected in the examples may be practiced, given the general description provided above. Although the foregoing invention has been described in some detail by way of illustration and example for purposes of clarity of understanding, the description and examples should not be construed as limiting the scope of the invention.

### Example 1

### Immunization of Camelids

Three camelids, i.e. one Llama (Lama glama), one Alpaca (Vicugna pacos) and one Huarizo (Lama glama×Vicugna pacos) were immunized with recombinant human (rh) NKp30 extracellular domain (ECD; produced in-house; see SEQ ID NO: 66) at preclinics GmbH, Germany. All procedures and animal care were in accordance with local animal welfare protection laws and regulation. In brief, 200 μg rh NKp30 diluted in 1 ml PBS were either emulsified with 1 ml Complete Freund's Adjuvant (initial immunization) or Incomplete Freund's Adjuvant (subsequent immunizations). Administrations were performed subcutaneously at three sites. A total of six immunizations (d0, d28, d42, d56, d70 and d84) were performed over the course of 84 days. On day 88, a volume of 100 ml blood was collected, total RNA was extracted, and cDNA was synthesized. All animals in this study were provided by preclinics GmbH and remained alive after completion of the immunization procedure.

### Yeast Strains and Media

For yeast surface display Saccharomyces cerevisiae strain EBY100 (MATa URA3-52 trp1 leu2Δ1 his34200 pep4::HIS3 prb1Δ1.6R can1 GAL (pIU211:URA3)) (Thermo Fisher Scientific) was utilized. Cells were cultivated in YPD medium composed of 20 g/L peptone, 20 g/L dextrose and 10 g/L yeast extract supplemented with 10 ml/l penstrep (Gibco). Cells harboring library plasmids (pDisp) after homologous recombination based cloning were cultivated in medium using minimal SD-base (Clontech) with commercially available dropout mix (Clontech) composed of all essential amino acids except for tryptophan (−Trp), according to the manufacturer's instructions, supplemented with 5.4 g/L Na2HPO4 and 8.6 g/L NaH2PO4×H2O. For induction of antibody gene expression cells were transferred into SG dropout medium (−Trp) wherein glucose was replaced by galactose containing SG-base (Clontech) supplemented with 10% (w/v) polyethylene glycol 800 (PEG 8000).

### Plasmids for Yeast Surface Display and Library Generation

Homologous recombination in yeast, referred to as gap repair cloning, was exploited for the generation of VHH sublibraries. PCR amplification of VHH fragments as well as library construction were carried out as described in Roth et al., 2020. In short, display plasmid pDisp was digested with BsaI followed by genetic fusion of VHH library candidates in frame to Aga2p by replacement of a stuffer sequence due to gap repair cloning, ultimately enabling surface presentation of sdAb variants on yeast cells. Furthermore, insertion of an HA epitope linked to the C-terminus of Aga2p allowed for the detection of full-length VHHs on the yeast surface (FIG. 1A).

### Library Sorting

For library sorting, rh his-tagged NKp30 ECD was purchased from Abcam. EBY100 library cells were grown overnight in SD medium with dropout mix lacking tryptophan (−Trp) at 30° C. and 120 rpm. For induction of surface expression, cells were transferred into SG medium with dropout mix (−Trp) at 10⁷ cells/ml followed by 48 h incubation at 20° C. Antigen binding was monitored by indirect immunofluorescence using his-tagged NKp30 followed by applying an anti-his mouse monoclonal detection antibody (SureLight® Allophycocyanin, Abcam, diluted 1:20). Full-length VHH surface expression was detected simultaneously by HA epitope labeling utilizing a FITC-labeled rabbit polyclonal antibody (Abcam, diluted 1:20). For the detection and isolation of library candidates, a BD FACSAria™ Fusion cell sorter (BD Biosciences) was employed. Control samples, i.e. untreated cells, cells incubated with labeling reagents only or cells incubated with labeling reagents and his-tagged, NKp30 or unrelated antigen were employed in every experiment, allowing for gate adjustment of the desired cell population.

### Antibody Expression and Purification

After sequencing and VHH clone selection, variants were fused to the hinge region of the SEED AG chain and cloned into pTT5 allowing for bispecific SEEDbody production in combination with humanized Cetuximab Fab on the SEED GA chain (FIG. 1A). SEEDbodies were either produced with a wild-type IgG1 CH2 domain (effector competent, VHH SEEDbody eff+) or in an effector silenced backbone by introduction of point mutations in the Fc region that abolish immune effector functions (VHH SEEDbody eff-). To this end, Expi293 cells were transfected with respective expression vectors according to the manufacturer's instructions (Thermo Fisher Scientific). Five days post expression, supernatants were harvested by centrifugation and purified via MabSelect antibody purification chromatography resin (GE Healthcare). Finally, buffer was exchanged to PBS pH 6.8 overnight using Pur-A-Lyzer™ Maxi 3500 Dialysis Kit (Sigma Aldrich). Concentrations were determined afterwards using Nanodrop ND-1000 (Peqlab) after sterile filtration with Ultrafree®-CL GV 0.22 μm centrifugal devices (Merck Millipore). Sample purities were assessed by determining target monomer peaks [%] via analytical size exclusion chromatography using 10 μg protein per sample, on a TSKgel SuperSW3000 column (4.6×300 mm, Tosoh Bioscience LLC) in an Agilent HPLC system with a flow rate of 0.35 ml/min.

### Biolayer Interferometry (BLI)

The Octet RED96 system (ForteBio, Pall Life Science) was employed for kinetic measurements as well as competition assays at 25° C. and 1000 rpm agitation. For binding kinetic measurements, bispecific SEEDbodies were loaded on anti-human Fc (AHC) Biosensors at 5 μg/mL in PBS for 3 min followed by 60 s sensor rinsing in kinetics buffer (KB; PBS, 0.1% Tween-20 and 1% bovine serum albumin, BSA). Afterwards, association to human NKp30 (Abcam) in varying concentrations ranging from 6.25 nM to 200 nM in KB was measured for 300 s followed by dissociation for 300 s (in KB). In each experiment, one negative control was measured using irrelevant antigen. Moreover, one reference value was measured incubating the antibody in KB instead of the antigen. In order to analyze competition for binding to NKp30 with the natural ligand B7-H6, NKp30 was loaded at 3 μg/mL in PBS for 3 min to anti-his tips (HIS1K) followed by 60 s sensor rinsing in KB. Association of the VHH SEEDbodies (200 nM) was conducted for 300 s (in KB), followed by an additional association step for 60 s with the natural ligand B7-H6 (500 nM, expressed as SEEDbody fusion) in KB. Epitope binning experiments of VHH SEEDbodies were conducted similarly except for using 200 nM for both VHH SEEDbody association steps for 180 s (first association) and 120 s (second association) in KB.

Data was fitted and analyzed with ForteBio data analysis software 8.0 using a 1:1 binding model after Savitzky-Golay filtering.

### Cell Culture

EGFR-expressing tumor cell lines A431 and A549 were obtained from DSMZ and cultured in RPMI 1640 Glutamax-I or Dulbecco's Modified Eagle's medium supplemented with 10% FCS, 100 U/ml penicillin and 100 mg/ml streptomycin (R10+ and D10+; all components from Thermo Fisher Scientific), respectively. Additionally, chinese hamster ovarian cells (ExpiCHO, Thermo Fisher Scientific) were cultivated in suspension with complete ExpiCHO Expression Medium.

### Tumor Cell Killings Assays

Preparation of PBMCs from healthy donors was performed as previously described after receiving written informed consent (Repp et al., 2011). NK cells were isolated by negative selection using NK cell isolation kit (Miltenyi Biotech) and maintained overnight at a density of 2×10⁶ cells/ml in R10+ medium. Cytotoxicity was analyzed in standard 4 h ⁵¹Cr release assays performed in 96-well microtiter plates in a total volume of 200 μl as described in Repp et al., 2011. Human PBMCs or purified NK cells were used as effector cells at effector-to-target cell (E:T) ratios of 80:1 and 10:1, respectively. VHH SEEDbodies or Cetuximab were applied at concentrations indicated.

### Cytokine Release Assay

Quantification of IFN-γ and TNF-α released by NK cells was performed using human cytokine HTRF kits (cisbio) as described in Pekar et al., 2021. In brief, 2.500 viable EGFR-positive A431 cells per well or EGFR-negative CHO cells were seeded in 384 clear bottom microtiter plates (Greiner Bio-One) and incubated for 3 h. 12.500 viable NK cells were added after overnight incubation in complete medium containing 100 U/ml recombinant human interleukin-2 (R&D systems), resulting in an effector cell to target cell (E:T) ratio of 5:1. SEEDbodies were added to a final concentration of 50 nM. As controls, tumor cells only as well as NK cells cultivated with tumor cells in absence of NK cell engagers were utilized. After 24 h incubation, cells were sedimented by centrifugation and cytokine containing supernatants were further processed according to manufacturer's instructions. Assay plates were measured with a PHERAstar FSX device (BMG Labtech). HTRF optical entity using excitation at 337 nM and emission at 620 nM as well as 665 nM was utilized. Analyses and fitting of resulting data was facilitated by MARS software (v.3.32, BMG) enabling a 4 Parameter Logistic (4PL 1/y²) model fitting of the standard curve following kit manufacturer's instructions.

### NK Cell Activation Assay

For bispecific SEEDbody mediated NK cell activation, the early activation marker CD69 was detected for CD16^{high} and CD16^{low} CD56⁺ cells via flow cytometry. To this end, 20.000 tumor cells per well were seeded into a 96-well V-bottom microtiter plate (Thermo Fisher Scientific) and incubated for 3 h before addition of 100.000 NK cells (rested overnight in media supplemented with 100 U/ml rh IL-2), resulting in an effector to target ratio of E:T=5:1. VHH SEEDbodies with a final concentration of 50 nM were added prior to an incubation period of 24 h at 37° C. Afterwards, assay plates were washed twice with PBS+1% BSA, followed by an 1 h incubation step on ice with detection reagents i.e. LIVE/DEAD™ Fixable Near-IR Dead Cell Stain (Thermo Fisher Scientific), anti-human CD56 PE (Miltenyi Biotec), and anti-CD69 allophycocyanin conjugated (abcam). After repeated washing, cells were analyzed via flow cytometry using an Intellicyt® iQue® Screener Plus (Sartorius) system. For compensation of fluorochromes antibody capturing analysis beads (OneComp eBeads™ Compensation Beads, Thermo Fisher Scientific) were employed according to the manufacturer's instructions.

### Data Processing and Statistical Analysis

Graphical and statistical analyses were performed with GraphPad Prism 8 software. P-values were calculated employing repeated measures ANOVA and the Bonferroni or Tukey post-test as recommended, or the student's t-test when appropriate. p≤0.05 were regarded as statistically significant.

All methods in Examples 2 to 7 were carried out as described in Example 1.

### Example 2

Selection of a Diverse Panel of NKp30 Specific VHH Single Domain Antibodies (sdAbs)

In order to generate sdAbs targeting NKp30, one Llama (Lama glama), one Alpaca (Vicugna pacos) and one Huarizo (Lama glama×Vicugna pacos) were immunized with recombinant human NKp30 extracellular domain (ECD; produced in-house) (FIG. 1A). Based on PBMCs derived from whole blood a yeast surface display library was constructed per animal specimen, resulting in three sub-libraries with sizes in the range of approximately 5×108 independent clones, respectively. These libraries were subjected to fluorescence activated cell sorting (FACS)-based selections (FIG. 1B). To this end, a two-dimensional labeling strategy was exploited to concomitantly detect for functional VHH surface expression as well as NKp30 binding. In the first round of selection, each sub-library was sorted separately by FACS using a NKp30 concentration of 1 μM (FIG. 1C). Afterwards, the library output was combined for a second selection round with significantly reduced NKp30 concentration (100 nM) aimed at enhancing selection stringencies. Subsequently, 96 clones were sent out for sequencing resulting in 76 unique clones (FIG. 1D). From clonotyping the selection output based on CDR3 sequence diversity, 18 clones were chosen for reformatting and expressed as bispecific SEEDbodies harboring the humanized Fab arm derived from EGFR targeting therapeutic antibody cetuximab (Wong, 2005; Davis et al., 2010). For silencing Fc mediated effector functions (eff-), specific amino acid exchanges were introduced into both heavy chains of the bispecific antibody derivatives.

### Example 3

Generated NK Cell Engagers Based on NKp30 Targeting sdAbs Demonstrate a Wide Range of Affinities and a Broad Epitope Coverage

Besides two SEEDbodies showing no productivity at all, expression yields for bispecific NK cell engagers were in the double to triple digit milligram per liter scale. Moreover, as shown in Table 1 below, aggregation properties as indicated by analytical size exclusion chromatography (SEC) post Protein A purification were fairly favorable i.e. for most of the molecules above 90% target species.

TABLE 1
Biochemical and biophysical characterization of VHH based NK cell engagers.

| | | | | SEC | Competition | Epitope |
|---|---|---|---|---|---|---|
| Name | KD (M) | kₒₙ (1/Ms) | k_{off} (1/s) | (%) | with B7-H6 | bin |
| VHH1 SEEDbody eff- | 2.70E−10 | 8.19E+05 | 2.22E−04 | 88 | yes | I |
| VHH2 SEEDbody eff- | 3.27E−09 | 1.98E+05 | 6.46E−04 | 90 | no | II |
| VHH3 SEEDbody eff- | 2.81E−10 | 6.04E+05 | 1.70E−04 | 92 | yes | I |
| VHH4 SEEDbody eff- | 2.57E−09 | 8.48E+04 | 2.18E−04 | 92 | partially | III |
| VHH5 SEEDbody eff- | 2.89E−10 | 6.43E+05 | 1.86E−04 | 93 | yes | I |
| VHH6 SEEDbody eff- | 2.92E−07 | 4.42E+05 | 1.29E−01 | 93 | no | IV |
| VHH7 SEEDbody eff- | 5.24E−08 | 6.72E+04 | 3.52E−03 | 86 | no | V |
| VHH8 SEEDbody eff- | 3.22E−10 | 4.16E+05 | 1.34E−04 | 96 | no | VI |
| VHH9 SEEDbody eff- | 4.99E−09 | 3.76E+05 | 1.87E−03 | 96 | no | VI |
| VHH10 SEEDbody eff- | 2.53E−10 | 3.46E+05 | 8.75E−05 | 91 | no | VI |
| VHH11 SEEDbody eff- | 1.02E−09 | 4.05E+05 | 4.15E−04 | 92 | no | VI |
| VHH12 SEEDbody eff- | 9.82E−10 | 9.14E+05 | 8.98E−04 | 91 | yes | I |
| VHH13 SEEDbody eff- | 2.11E−10 | 7.66E+05 | 1.62E−04 | 90 | yes | I |
| VHH14 SEEDbody eff- | 3.10E−10 | 4.64E+05 | 1.44E−04 | 97 | yes | I |
| VHH15 SEEDbody eff- | <1.0E−12 | 4.40E+05 | <1.0E−07 | 90 | yes | I |
| VHH16 SEEDbody eff- | 1.39E−10 | 6.66E+05 | 9.28E−05 | 91 | yes | I |

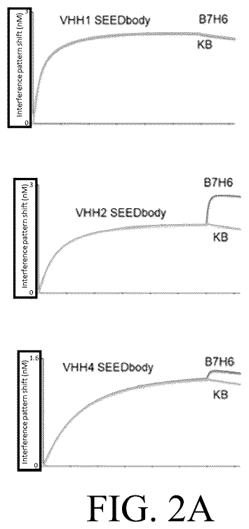

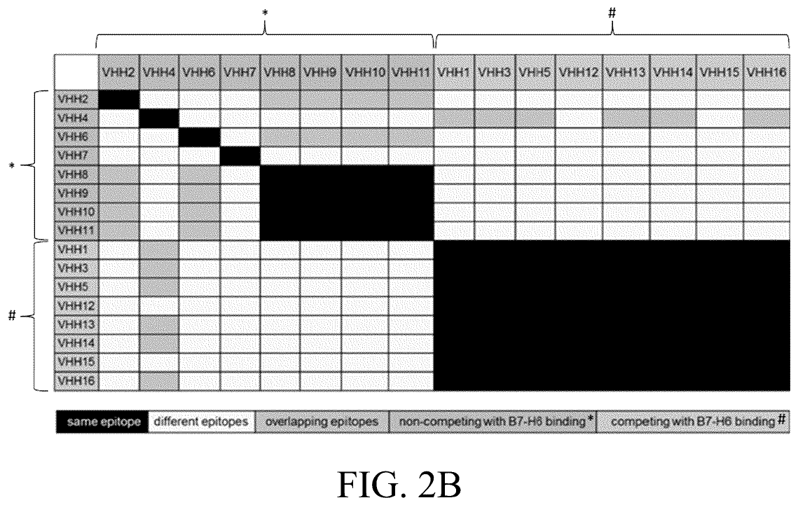

Intriguingly, the bispecific antibodies displayed a broad range of affinities with respect to binding to NKp30, ranging from triple digit nanomolar binding (VHH6 SEEDbody) to affinities in the sub-nanomolar range (VHH1, VHH3, VHH5, VHH8, VHH10, VHH12-VHH16 SEEDbodies). Epitope specificity was determined using biolayer interferometry (BLI). In a first assay, competition of VHH SEEDbodies with the natural ligand B7-H6 for binding to NKp30 was assessed. To this end, recombinant NKp30 ECD was captured to the biosensor. Subsequently, an association step was performed employing the respective VHH SEEDbody followed by incubation with the N-terminal V-like domain of B7-H6 produced as SEEDbody fusion (Pekar et al., 2021). Overall, eight VHH SEEDbodies showed competition with B7-H6 for NKp30 binding, indicating that these molecules share the same epitope bin with the natural ligand (Table 1). One partially competing molecule was identified (VHH4 SEEDbody) as well as seven non-competing moieties. FIG. 2A shows representative BLI sensograms for a B7-H6 competitor (VHH1 SEEDbody), a non-competitor (VHH2 SEEDBody) as well as the partial competitor (VHH4 SEEDbody). Moreover, to scrutinize epitope coverage more meticulously, pairwise competition was performed using all VHH SEEDbodies in every possible combination (Table 1, FIG. 2B). These experiments revealed that all B7-H6 competing VHH SEEDbodies mutually prevented binding and, therefore, share very similar epitopes on NKp30. Additionally, most of them share an overlapping epitope with VHH4 SEEDbody, which partially competes with B7-H6 binding to NKp30. However, VHH12 and VHH15 SEEDbodies did not compete for binding with VHH4 SEEDbody, indicating also subtle differences of epitope targeting within this set of B7-H6 competitors. Among the B7-H6 noncompeting bispecifics, SEEDbodies harboring VHH8, VHH9, VHH10 and VHH11 share the same epitope bin that partially overlaps with the bins of VHH2 and VHH6 SEEDbodies. Moreover, VHH2, VHH6 and VHH7 SEEDbodies were unique with respect to epitope targeting. Taken together, the generated VHH SEEDbodies display a broad epitope diversity represented by seven epitope bins.

### Example 4

### Killing of EGFR-Positive Tumor Cells by VHH SEEDbodies is Significantly Influenced by the NKp30 Binding Epitope

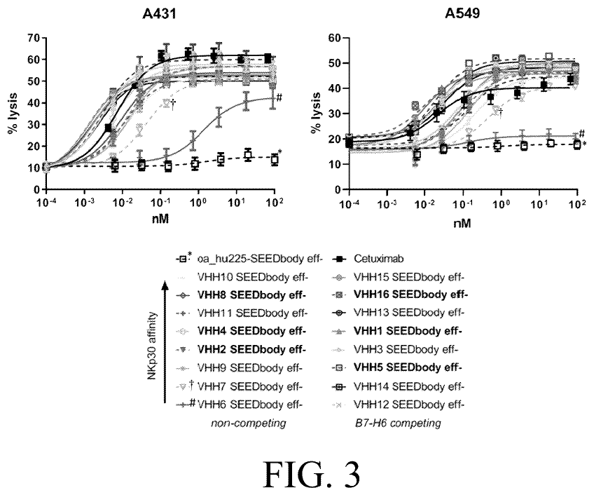

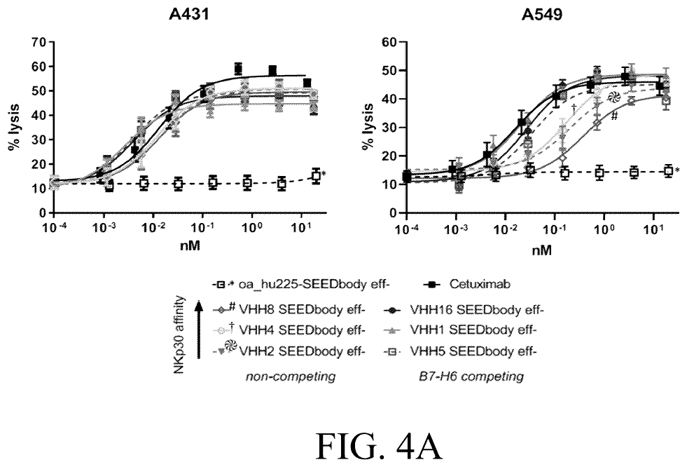

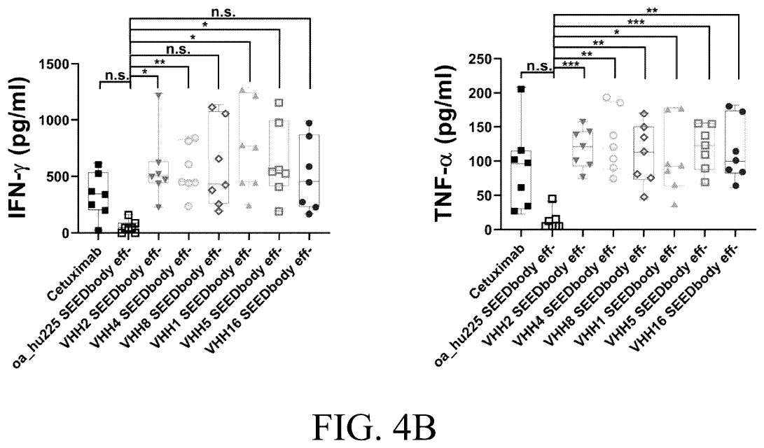

Initial functional analyses of the NK cell engaging VHH SEEDbodies was performed with EGFR-expressing tumor cell lines A431 and A549 using mononuclear cells (MNC) of healthy donors as effector cells in standard chromium release assays. Interestingly, the B7-H6 competitors and non-competitors clustered in two groups, with the B7-H6 competing VHH SEEDbodies mediating more efficient lysis of tumor cells. This was true for the high EGFR-expressing cell line A431 and even more pronounced for the lower EGFR-expressing cell line A549 (FIG. 3). Interestingly, VHH6 SEEDbody having the lowest affinity for NKp30 (292 nM) and not interfering with the binding site of the natural ligand B7-H6 elicited the weakest killing of A431 cells with respect to potencies (FIG. 3). Moreover, almost no killing of A549 tumor cells was observed for this molecule. This is in stark contrast to all eight VHH SEEDbodies that compete with B7-H6 for NKp30 binding. These molecules induced more potent killing than the monoclonal antibody cetuximab irrespective of a higher or lower EGFR expression on the tumor cells (FIG. 3), while activating NK cells exclusively via NKp30. Based on these results, the best three B7-H6 competing SEEDbodies (VHH1, VHH5, VHH16 SEEDbodies) as well as the best three non-competing SEEDbodies (VHH2, VHH4, VHH8 SEEDbodies) were chosen for further characterization, including VHH4 SEEDbody as partial competitor. To this end, tumor cell killing assays with A431 and A549 cells using freshly isolated NK cells were performed (FIG. 4A; Table 2). Ultimately, B7-H6 competitors were more potent than B7-H6 non-competitors with respect to tumor cell killing particularly using lower EGFR-positive tumor cell line A549, clearly confirming dependencies in killing capacities of VHH-based NKCEs based on the targeted epitope on NKp30.

In a second experiment studying the induced tumor cell death due to natural killer cell activation, EC50 and maximal killing was determined for MNC based killing assays using A431 cells as summarized in Table 2.

TABLE 2

| | | Max. Killing |
|---|---|---|
| Compound | EC50 (nM) | (% Lysis) |
| VHH1 SEEDbody eff- | 0.001714 | 53 |
| VHH2 SEEDbody eff- | 0.01319 | 51 |
| VHH3 SEEDbody eff- | 0.002843 | 53 |
| VHH4 SEEDbody eff- | 0.005917 | 55 |
| VHH5 SEEDbody eff- | 0.002859 | 56 |
| VHH6 SEEDbody eff- | 1.425 | 42 |
| VHH7 SEEDbody eff- | 0.04333 | 49 |
| VHH8 SEEDbody eff- | 0.008987 | 50 |
| VHH9 SEEDbody eff- | 0.01612 | 53 |
| VHH10 SEEDbody eff- | 0.01463 | 51 |
| VHH11 SEEDbody eff- | 0.01108 | 50 |
| VHH12 SEEDbody eff- | 0.002069 | 49 |
| VHH13 SEEDbody eff- | 0.002406 | 51 |
| VHH14 SEEDbody eff- | 0.00144 | 51 |
| VHH15 SEEDbody eff- | 0.001305 | 50 |
| VHH16 SEEDbody eff- | 0.001275 | 50 |

Effector cell to target cell ratio for MNC based killing assays = 80:1. Average values shown based on independent experiments of three healthy donors.

### Example 5

### VHH SEEDbodies Induce Potent Release of Proinflammatory Cytokines TNF-α and INF-γ

In addition, all scrutinized VHH SEEDbodies promoted significant NK cell production of TNF-α and INF-γ in a target dependent manner (FIG. 4B). In this respect, capabilities in eliciting the release of proinflammatory cytokines were not appreciably different between the group of B7-H6 non-competing and competing molecules. Intriguingly, a trend towards elevated levels of TNF-α and INF-γ release was also observed in direct comparison to therapeutic antibody Cetuximab. While Cetuximab triggered the release of 90.4 pg/ml TNF-α and 336.7 pg/ml INF-γ on average (of note, capacities of Cetuximab were not statistically significant higher compared to a monovalent EGFR-targeting effector-silent SEEDbody control with 8.6 pg/ml TNF-α and 53.8 pg/ml INF-γ on average) VHH SEEDbodies promoted NK cell mediated release of 103.6-129.2 pg/ml TNF-α as well as 514.6-752.6 pg/ml INF-γ, clearly indicating a differentiated cytokine release profile.

TABLE 3

| | Max. TNFα | Max. IFNγ |
|---|---|---|
| | release | release |
| Compound | (pg/ml)^{#} | (pg/ml)^{#} |
| VHH1 SEEDbody eff- | 103.6 | 752.6 |
| VHH2 SEEDbody eff- | 118.0 | 579.8 |
| VHH4 SEEDbody eff- | 129.0 | 559.5 |
| VHH5 SEEDbody eff- | 119.1 | 635.0 |
| VHH8 SEEDbody eff- | 109.3 | 594.6 |
| VHH16 SEEDbody eff- | 114.1 | 514.6 |

^{#}Values are calculated averages of seven donors (n = 2).

### Example 6

Soluble B7-H6 does not Impair Tumor Cell Killing Induced by VHH SEEDbodies

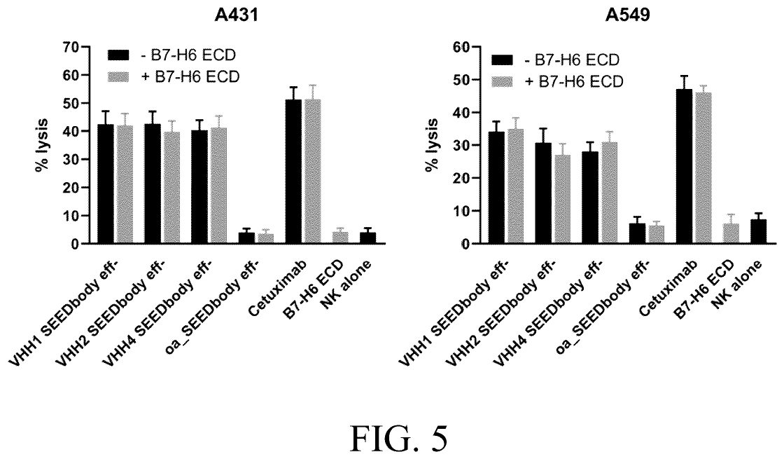

Cancer patients have remarkably higher soluble B7-H6 (sB7-H6) concentrations of up to 5 nM in their serum compared to healthy individuals (Rusakiewicz et al., 2017). In order to investigate, whether shed B7-H6 negatively impacts killing capacities of the generated NK cell engaging VHH SEEDbodies, we performed killing assays with the B7-H6 competing VHH1 SEEDbody, the partially competing VHH4 SEEDbody and the non-competing VHH2 SEEDbody in the presence or absence of high concentrations (178.6 nM) of the extracellular domain of B7-H6 (B7-H6 ECD-His; FIG. 5). Of note, the selected concentration of 178.6 nM B7-H6 was substantially higher than typically found in cancer patients in order to scrutinize and even amplify subtle effects that may result from soluble B7-H6 mediated receptor blockade. Interestingly, neither killing of A431 nor A549 cells through any of the three VHH SEEDbodies was affected by B7-H6 ECD, indicating that shed B7-H6 in cancer patients' serum most likely would not impair tumor cell killing mediated by the herein described NKCEs.

### Example 7

Additional FcγRIIIa engagement improves anti-tumor activities of VHH SEEDbodies

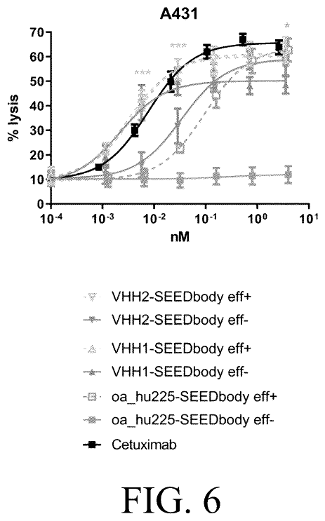

In an attempt to further enhance the cytotoxic potential of the B7-H6 competing VHH1 and non-competing VHH2 SEEDbodies, we also produced these molecules with an Fc region capable of binding to FcγRIIIa (SEEDbody lacking the mutations that abolish immune effector functions; eff+). As shown in FIG. 6, lysis of high EGFR-expressing A431 tumor cells mediated by B7-H6 competing VHH1 SEEDbody could only be improved with respect to killing efficacy i.e. maximal lysis by concomitant engagement of FcγRIIIa, while killing potencies remained unaffected (EC₅₀ VHH1 SEEDbody eff-=2.2 pM vs. EC₅₀ VHH1 SEEDbody eff+=3.2 pM). In contrast, the potency of B7-H6 non-competing VHH2 SEEDbody was significantly increased by the factor of 8.8 by incorporating an effector functional Fc region (EC₅₀ VHH2 SEEDbody eff-=34.4 pM vs. EC₅₀ VHH2 SEEDbody eff+=3.9 pM), whereas no significant differences were observed with respect to the maximum lysis rates achieved by both VHH2 SEEDbodies (FIG. 6).

Taken together, these data suggest that killing capacities of VHH-based NKCEs can be optimized by concomitant engagement of FcγRIIIa in one way or the other i.e. in terms of potencies or efficacies. This beneficial effect contributing to overall anti-tumor activities seems to be more pronounced for B7-H6 non-competing SEEDbodies that per se were not as potent in tumor cell killing.

### Example 8

### Humanized VHH1 Variants

Humanized variants of VHH1 (SEQ ID NO: 1) were prepared. In these humanized versions of VHH1, the CDRs are identical to those of VHH1, but compared to VHH1 individual amino acids in the framework regions are substituted by corresponding amino acids of a human germline sequence (VHH1.1 to VHH1.12, represented by SEQ ID NO: 67 to 78).

Constructs were expressed in small scale expression as described in Experiment 1. After purification (identity confirmed by mass validation), purified variants were analyzed by size exclusion chromatography (SEC) and their binding to NKp30 and KD value was determined by biolayer interferometry (BLI) measurements as described in Experiment 1.

Results are summarized in the table below:

| | | | SEC purity | | |
|---|---|---|---|---|---|
| | SEQ ID | Yield | (% target | | Emax |
| VHH | NO | (mg/L) | monomer peak) | KD [nM] | (nm) |
| VHH1 | 1 | 66.5 | 87.2 | 0.248 | 0.13 |
| VHH1.1 | 67 | 58.8 | 89.5 | 0.275 | 0.13 |
| VHH1.2 | 68 | 41.9 | 85.3 | 0.293 | 0.12 |
| VHH1.3 | 69 | 38.3 | 85.4 | 1.07 | 0.12 |
| VHH1.4 | 70 | 86.3 | 76.8 | 0.547 | 0.12 |
| VHH1.5 | 71 | 87.3 | 73 | 0.888 | 0.10 |
| VHH1.6 | 72 | 74.1 | 63.8 | 0.797 | 0.14 |
| VHH1.7 | 73 | 80.7 | 65 | 0.533 | 0.09 |
| VHH1.8 | 74 | 70.0 | 73 | 1.13 | 0.10 |
| VHH1.9 | 75 | 68.4 | 69.8 | 1.06 | 0.10 |
| VHH1.10 | 76 | 85.2 | 64.4 | nb | nb |

nb: no binding or only weak binding to NKp30 target antigen in BLI assay at concentration ≤100 nM

As can be seen from these data, all of humanized variants VHH1.1 to VHH1.9 show in vitro properties that are similar to VHH1 in terms expression yield/purity, KD, binding capacity, and thus expected functionality. Of these, VHH1.1 to VHH1.3 are particularly well aligned with VHH1 with respect to their in vitro properties. Only VHH1.10 shows less favorable overall in vitro properties (KD and maximum binding capacity).

### Example 9

### Humanized VHH2 Variants

Humanized variants of VHH2 (SEQ ID NO: 2) were prepared. In these humanized versions of VHH2, the CDRs are identical to those of VHH2, but compared to VHH2 individual amino acids in the framework regions are substituted by corresponding amino acids of a human germline sequence (VHH2.1 to VHH2.18, represented by SEQ ID NO: 79 to 96).

Constructs were expressed in small scale expression as described in Experiment 1. After purification (identity confirmed by mass validation), purified variants were analyzed by size exclusion chromatography (SEC) and their binding to NKp30 and KD value was determined by biolayer interferometry (BLI) measurements as described in Experiment 1.

Results are summarized in the table below:

| | | | SEC purity | | |
|---|---|---|---|---|---|
| | SEQ ID | Yield | (% target | | Emax |
| VHH | NO | (mg/L) | monomer peak) | KD [nM] | (nm) |
| VHH2 | 2 | 98.2 | 89.1 | 2.88 | 0.17 |
| VHH2.1 | 79 | 104.7 | 89.5 | 2.91 | 0.18 |
| VHH2.2 | 80 | 120.2 | 89.5 | 2.1 | 0.15 |
| VHH2.3 | 81 | 43.1 | 97.3 | 9.97 | 0.13 |
| VHH2.4 | 82 | 26.1 | 100 | nb | nb |
| VHH2.5 | 83 | 86.6 | 95.7 | 2.92 | 0.11 |
| VHH2.6 | 84 | 77.6 | 94 | 27.6 | 0.07 |
| VHH2.7 | 85 | 63.1 | 96.9 | 9.76 | 0.1 |
| VHH2.8 | 86 | 109.9 | 94.4 | 6.61 | 0.1 |
| VHH2.9 | 87 | 37.5 | 98.1 | nb | nb |
| VHH2.10 | 88 | 42.3 | 97.9 | nb | nb |
| VHH2.11 | 89 | 100.1 | 93.5 | 4.7 | 0.16 |
| VHH2.12 | 90 | 118.6 | 93.3 | 4.24 | 0.14 |
| VHH2.13 | 91 | 98.6 | 95.8 | nb | nb |
| VHH2.14 | 92 | 82.1 | 95.9 | nb | nb |
| VHH2.15 | 93 | 21.5 | 99.1 | 12.3 | 0.07 |
| VHH2.16 | 94 | 86.1 | 97.8 | 4.71 | 0.11 |
| VHH2.17 | 95 | 18.0 | 100 | nb | nb |
| VHH2.18 | 96 | 91.9 | 97.1 | nb | nb |

nb: no binding or only weak binding to NKp30 target antigen in BLI assay at concentration ≤100 nM

As can be seen from these data, all of humanized variants VHH2.1, VHH2.2, VHH2.3, VHH2.5, VHH2.6, VHH2.7, VHH2.8, VHH2.11, VHH2.12, VHH2.15 and VHH2.16 show in vitro properties that are similar to VHH1 in terms expression yield/purity, KD, binding capacity, and thus expected functionality. Of these, VHH2.1, VHH2.2, VHH2.5, VHH2.8, VHH2.11, VHH2.12 and VHH2.16 are particularly well aligned with VHH2 with respect to their in vitro properties. VHH2.4, VHH2.9, VHH2.10, VHH2.13, VHH2.14, VHH2.17 and VHH2.18 show less favorable overall in vitro properties (KD and maximum binding capacity).

### REFERENCES

P. André et al., “Anti-NKG2A mAb Is a Checkpoint Inhibitor that Promotes Anti-tumor Immunity by Unleashing Both T and NK Cells,” Cell (2018), vol. 175, no. 7, pp. 1731-1743.e13
D. M. Benson et al., “A Phase I Trial of the Anti-KIR Antibody IPH2101 and Lenalidomide in Patients with Relapsed/Refractory Multiple Myeloma,” Clinical Cancer Research (2015), vol. 21, no. 18, pp. 4055-4061
Y. T. Bryceson, M. E. March, D. F. Barber, H.-G. Ljunggren, and E. O. Long, “Cytolytic granule polarization and degranulation controlled by different receptors in resting NK cells,” The Journal of Experimental Medicine (2005), vol. 202, no. 7, pp. 1001-1012
M. C. Burger et al., “CAR-Engineered NK Cells for the Treatment of Glioblastoma: Turning Innate Effectors Into Precision Tools for Cancer Immunotherapy,” Frontiers in Immunology (2019), vol. 10, p. 2683
M. Carlsten and M. Järås, “Natural Killer Cells in Myeloid Malignancies: Immune Surveillance, NK Cell Dysfunction, and Pharmacological Opportunities to Bolster the Endogenous NK Cells,” Frontiers in Immunology (2019), vol. 10, p. 2357
L. Chiossone, P.-Y. Dumas, M. Vienne, and E. Vivier, “Natural killer cells and other innate lymphoid cells in cancer,” Nature Reviews Immunology (2018), vol. 18, no. 11, pp. 671-688
E. Coligan et al., “Current Protocols in Protein Science”, vol. 2 (1997), John Wiley & Sons, Inc.
V. Cortez-Retamozo et al., “Efficient tumor targeting by single-domain antibody fragments of camels,” Int J Cancer (2002), vol. 89, pp. 456-462
J. H. Davis et al., “SEEDbodies: fusion proteins based on strand-exchange engineered domain (SEED) CH3 heterodimers in an Fc analogue platform for asymmetric binders or immunofusions and bispecific antibodies,” Protein Engineering, Design and Selection (2010), vol. 23, no. 4, pp. 195-202
M. Dumoulin et al., “A camelid antibody fragment inhibits the formation of amyloid fibrils by human lysozyme,” Nature (2003), vol. 424, pp. 783-788
K. Ellwanger et al., “Redirected optimized cell killing (ROCK®): A highly versatile multispecific fit-for-purpose antibody platform for engaging innate immunity,” mAbs (2019), vol. 11, no. 5, pp. 899-918
L. Gauthier et al., “Multifunctional Natural Killer Cell Engagers Targeting NKp46 Trigger Protective Tumor Immunity,” Cell (2019), vol. 177, no. 7, pp. 1701-1713.e16
Gonzalez-Rodriguez, Villa-Álvarez, Sordo-Bahamonde, Lorenzo-Herrero, and Gonzalez, “NK Cells in the Treatment of Hematological Malignancies,” Journal of Clinical Medicine (2019), vol. 8, no. 10, p. 1557
Green and Sambrook, “Molecular Cloning: A Laboratory Manual”, 4th ed. (2014), Cold Spring Harbor Laboratory Press (U.S.A.)
C. Kellner et al., “Enhancing natural killer cell-mediated lysis of lymphoma cells by combining therapeutic antibodies with CD20-specific immunoligands engaging NKG2D or NKp30,” OncoImmunology (2016), vol. 5, no. 1, p. e1058459
J. Koch and M. Tesar, “Recombinant Antibodies to Arm Cytotoxic Lymphocytes in Cancer Immunotherapy,” Transfusion Medicine and Hemotherapy (2017), vol. 44, no. 5, pp. 337-350
D. Könning et al., “Camelid and shark single domain antibodies: structural features and therapeutic potential,” Curr Opin Struct Biol. (2017), vol. 45, pp. 10⁻¹⁶
H. E. Kohrt et al., “Anti-KIR antibody enhancement of anti-lymphoma activity of natural killer cells as monotherapy and in combination with anti-CD20 antibodies,” Blood (2014), vol. 123, no. 5, pp. 678-686
M. Lauwereys et al., “Potent enzyme inhibitors derived from dromedary heavy-chain antibodies,” EMBO J (1998), vol. 17. pp. 3512-3520
S. Morgado et al., “NK Cell Recognition and Killing of Melanoma Cells Is Controlled by Multiple Activating Receptor-Ligand Interactions,” Journal of Innate Immunity (2011), vol. 3, no. 4, pp. 365-373
M. Peipp et al., “HER2-specific immunoligands engaging NKp30 or NKp80 trigger NK-cell-mediated lysis of tumor cells and enhance antibody-dependent cell-mediated cytotoxicity,” Oncotarget (2015), vol. 6, no. 31
L. Pekar et al., “Affinity Maturation of B7-H6 Translates into Enhanced NK Cell-Mediated Tumor Cell Lysis and Improved Proinflammatory Cytokine Release of Bispecific Immunoligands via NKp30 Engagement,” The Journal of Immunology (2021), vol. 206, no. 1, pp. 225-236
M. Pleschberger et al., “Generation of a functional monomolecular protein lattice consisting of an s-layer fusion protein comprising the variable domain of a camel heavy chain antibody,” Bioconjugate Chem (2003), vol. 14: 440-448
R. Repp et al., “Combined Fc-protein- and Fc-glyco-engineering of scFv-Fc fusion proteins synergistically enhances CD16a binding but does not further enhance NK-cell mediated ADCC,” Journal of Immunological Methods (2011), vol. 373, pp. 67-78
K. Rezvani, “Adoptive cell therapy using engineered natural killer cells,” Bone Marrow Transplantation (2019), vol. 54, no. S2, pp. 785-788
L. Roth et al., “Isolation of Antigen-Specific VHH Single-Domain Antibodies by Combining Animal Immunization with Yeast Surface Display,” Methods Mol. Biol. (2020), vol. 2070, pp. 173-189
A. Rothe et al., “A phase 1 study of the bispecific anti-CD30/CD16A antibody construct AFM13 in patients with relapsed or refractory Hodgkin lymphoma,” Blood (2015), vol. 125, no. 26, pp. 4024-4031
S. Rusakiewicz et al., “NKp30 isoforms and NKp30 ligands are predictive biomarkers of response to imatinib mesylate in metastatic GIST patients,” OncoImmunology (2017), vol. 6, no. 1, p. e1137418
U. J. E. Seidel, P. Schlegel, and P. Lang, “Natural Killer Cell Mediated Antibody-Dependent Cellular Cytotoxicity in Tumor Immunotherapy with Therapeutic Antibodies,” Frontiers in Immunology (2013), vol. 4
C. Sellmann et al., “A One-Step Process for the Construction of Phage Display scFv and VHH Libraries,” Mol. Biotechnol. (2020), vol. 62(4), pp. 228-239
E. P. von Strandmann et al., “A novel bispecific protein (ULBP2-BB4) targeting the NKG2D receptor on natural killer (NK) cells and CD138 activates NK cells and has potent antitumor activity against human multiple myeloma in vitro and in vivo,” Blood (2006), vol. 107, no. 5, pp. 1955-1962
E. Vivier, E. Tomasello, M. Baratin, T. Walzer, and S. Ugolini, “Functions of natural killer cells,” Nat. Immunol. (2008), vol. 9, no. 5, pp. 503-510
S.-F. Wong, “Cetuximab: An epidermal growth factor receptor monoclonal antibody for the treatment of colorectal cancer,” Clinical Therapeutics (2005), vol. 27, no. 6, pp. 684-694

1. A compound, comprising:
a VHH antibody domain or a fragment thereof, wherein
(a) said VHH antibody domain or fragment thereof comprises the complementarity determining regions CDR1, CDR2 and CDR3 of one of VHH1 to VHH16 as shown in the Table of CDRs below;
(b) said VHH antibody domain or fragment thereof comprises the complementarity determining regions CDR1, CDR2 and CDR3 as defined in (a) with modification, wherein the modification is that the sequence of at least one of CDR1, CDR2 and CDR3 is humanized; or
(c) said VHH antibody domain or fragment thereof comprises the complementarity determining regions CDR1, CDR2 and CDR3 as defined in (a) with modification, wherein the modification is
the replacement, addition or deletion of up to three amino acids in CDR1;
the replacement, addition or deletion of up to three amino acids in CDR2; and/or
the replacement, addition or deletion of up to three amino acids in CDR3;
Table of CDRs:

| | CDR1 | CDR2 | CDR3 |
|---|---|---|---|
| VHH1 | ATSGFTLDDY | CFSPSD | AASFADGSSW |
| | TIG | GTTY | CYDYW |
| | (SEQ ID | (SEQ ID | (SEQ ID |
| | NO: 18) | NO: 19) | NO: 20) |
| VHH2 | AVSGQTWTNY | SIEWGGRGTY | AAQSSSRSPL |
| | HIG | A | ESNYDYW |
| | (SEQ ID | (SEQ ID | (SEQ ID |
| | NO: 21) | NO: 22) | NO: 23) |
| VHH3 | LVSGVTYHFY | CISYASGTTK | ATQGSGSCSD |
| | DIT | F | FMSAGSVLSW |
| | (SEQ ID | (SEQ ID | (SEQ ID |
| | NO: 24) | NO: 25) | NO: 26) |
| VHH4 | LVSGVTYHFY | CISYASGSTK | AMQGSGSCSD |
| | DIT | F | FMSADSVLSW |
| | (SEQ ID | (SEQ ID | (SEQ ID |
| | NO: 27) | NO: 28) | NO: 29) |
| VHHS | LVSGVTYHFY | CISYASGSTK | ATQGSGSCSD |
| | DIT | F | FMSADSVLSW |
| | (SEQ ID | (SEQ ID | (SEQ ID |
| | NO: 30) | NO: 31) | NO: 32) |
| VHH6 | AASGLPFSKY | VVSWSG | AASGSYPVTD |
| | AIA | DKFY | RYFYNFW |
| | (SEQ ID | (SEQ ID | (SEQ ID |
| | NO: 33) | NO: 34) | NO: 35) |
| VHH7 | AASGFTFSSY | AAIFSAGGST | NKAPPFTTIQ |
| | DMG | IYA | PTCVMIDTQL |
| | (SEQ ID | (SEQ ID | GAW |
| | NO: 36) | NO: 37) | (SEQ ID |
| | | | NO: 38) |
| VHH8 | AASGFTFSSY | AAIFSADGST | NKAPPFTTIQ |
| | DMG | IYA | PMCVMIDTQL |
| | (SEQ ID | (SEQ ID | GAW |
| | NO: 39) | NO: 40) | (SEQ ID |
| | | | NO: 41) |
| VHH9 | AASGRGGASY | AAISWTGDTT | AAAHWTRVVA |
| | AMG | YN | KGAMADEKKY |
| | (SEQ ID | (SEQ ID | EYW |
| | NO: 42) | NO: 43) | (SEQ ID |
| | | | NO: 44) |
| VHH10 | AASGRTFSSN | AAIHWSGGSR | AAEISWQTTG |
| | FMG | AYA | WTNLGSW |
| | (SEQ ID | (SEQ ID | (SEQ ID |
| | NO: 45) | NO: 46) | NO: 47) |
| VHH11 | AASGRTFSSD | AAIHWSGGST | AAEIRWLTTG |
| | FMG | SYA | WANLGSW |
| | (SEQ ID | (SEQ ID | (SEQ ID |
| | NO: 48) | NO: 49) | NO: 50) |
| VHH12 | AASGRTFSSD | AAIHWSGGSR | AAEIRWQTTG |
| | FMG | SYA | WANLGSW |
| | (SEQ ID | (SEQ ID | (SEQ ID |
| | NO: 51) | NO: 52) | NO: 53) |
| VHH13 | AASGRTFTSD | AAIHWSGGSR | AAEIRWQTAG |
| | FMG | SYA | WVNLGSW |
| | (SEQ ID | (SEQ ID | (SEQ ID |
| | NO: 54) | NO: 55) | NO: 56) |
| VHH14 | EASGVTFNFY | ADITSEGRTE | YTGG |
| | SMT | YA | SW |
| | (SEQ ID | (SEQ ID | (SEQ ID |
| | NO: 57) | NO: 58) | NO: 59) |
| VHHIS | AVSGSTFRLN | AGITADGGVT | NTAPA |
| | VMG | NYA | NWGSW |
| | (SEQ ID | (SEQ ID | (SEQ ID |
| | NO: 60) | NO: 61) | NO: 62) |
| VHH16 | TASGSIFSSL | AGITTDGSTD | NKSPYYDPTD |
| | TMAW | YA | YDSW |
| | (SEQ ID | (SEQ ID | (SEQ ID |
| | NO: 63) | NO: 64) | NO: 65) |

2. The compound according to claim 1, wherein said compound comprises the sequence represented by SEQ ID NO: 1 (or a fragment thereof), with one or more amino acid substitutions compared to the sequence of SEQ ID NO: 1, wherein, if such amino acid substitutions are located outside of the CDRs, said amino acid substitution(s) are at least one selected from the group consisting off D1E, Q5V, L11V, T24A, E44G, H45L, G47W, F70I, V79L, K87R, P88A, and Q116L.

3. The compound according to claim 1, wherein said compound has a sequence represented by at least one SEQ ID NO selected from the group consisting of SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 77, and a fragment thereof.

4. The compound according to claim 1, wherein said compound comprises the sequence represented by SEQ ID NO: 2 or a fragment thereof, with one or more amino acid substitutions compared to the sequence of SEQ ID NO: 2, wherein, if such amino acid substitutions are located outside of the CDRs, said one or more amino acid substitutions are selected from the following: group consisting of D1E, Q5V, L11V, A14P, V24A, F37V, A44G, R45L, S47W, V79L, K87R, P88A, and Q118L.

5. The compound according to claim 1, wherein said compound comprises the sequence represented by at least one SEQ ID NO selected from the group consisting of SEQ ID NO: 79, SEQ ID NO: 80, SEQ ID NO: 81, SEQ ID NO: 83, SEQ ID NO: 84, SEQ ID NO: 85, SEQ ID NO: 86, SEQ ID NO: 89, SEQ ID NO: 90, SEQ ID NO: 93, SEQ ID NO: 94, and a fragment thereof.

6. A compound, comprising:
a VHH antibody domain or a fragment thereof, wherein
(A) said VHH antibody domain comprises any one of the VHH sequences VHH1 to VHH16 shown in the Table of VHH Sequences below;
(B) said VHH antibody domain comprises a VHH sequence as defined in (A) with modification, wherein the modification is that said sequence is humanized;
(C) said VHH antibody domain comprises a VHH sequence as defined in (A) with modification, wherein the modification is the replacement, addition or deletion of up to 25 amino acids; or
(D) said VHH antibody domain comprises a VHH sequence that is at least 75% identical to a VHH sequence referred to in (A);
Table of VHH Sequences:

| VHH Sequence | SEQ ID NO | Amino acid sequence |
|---|---|---|
| VHH1 | SEQ ID NO: 1 | DVQLQESGGGLVQPGGSLRLSCATSGFTLD |
| | | DYTIGWVRQAPGKEHEGVSCFSPSDGTTY |
| | | YADSVKGRFTFSRDNAKNTVYLQMNSLKP |
| | | EDTAVYYCAASFADGSSWCYDYWGQGTQ |
| | | VTVSS |
| VHH2 | SEQ ID NO: 2 | DVQLQESGGGLVQAGGSLRLSCAVSGQTW |
| | | TNYHIGWFRQAPGKARESVASIEWGGRGT |
| | | YATDSVKGRFTISRDNAKNTVYLQMNSLK |
| | | PEDTAVYYCAAQSSSRSPLESNYDYWGQG |
| | | TQVTVSS |
| VHH3 | SEQ ID NO: 3 | DVQLQESGGGLVQPGESLKLSCLVSGVTY |
| | | HFYDITWFRQAPGLEREGVSCISYASGTTK |
| | | FADSVKGRFTISRDNAKNAVYLEMNSLKPE |
| | | DTAVYFCATQGSGSCSDFMSAGSVLSWGQ |
| | | GTQVTVSS |
| VHH4 | SEQ ID NO: 4 | DVQLQESGGGLVQPGESLRLSCLVSGVTY |
| | | HFYDITWFRQAPGLEREGVSCISYASGSTK |
| | | FADSVKGRFTISRDNAKNAVYLQMNSLKPE |
| | | DTAVYFCAMQGSGSCSDFMSADSVLSWG |
| | | QGTQVTVSS |
| VHHS | SEQ ID NO: 5 | DVQLQESGGGLVQPGESLRLSCLVSGVTY |
| | | HFYDITWFRQAPGLEREGVSCISYASGST |
| | | KFADSVKGRFTISRDNAKNAVYLQMNSLK |
| | | PEDTAVYFCATQGSGSCSDFMSADSVLSW |
| | | GQGTQVTVSS |
| VHH6 | SEQ ID NO: 6 | DVQLQESGGGLVQTGGSLRLSCAASGLPFS |
| | | KYAIAWFRQAPGKESEFLVVVSWSGDKFY |
| | | QDNMKGRCTISRDNAKNTAYLQINSLQPE |
| | | DTAVYYCAASGSYPVTDRYFYNFWGQGT |
| | | QVTVSS |
| VHH7 | SEQ ID NO: 7 | DVQLQESGGGLAQPGGSLRLSCAASGFTFS |
| | | SYDMGWYRQVPGKECELVAAIFSAGGSTI |
| | | YADSVKGRFTISRDNAKNTVFLQMNSLKPE |
| | | DTAVYYCNKAPPFTTIQPTCVMIDTQLGA |
| | | WGQGTQVTVSS |
| VHHS | SEQ ID NO: 8 | DVQLQESGGGLAQPGGSLRLSCAASGFTFS |
| | | SYDMGWYRQAPGKECELVAAIFSADGSTI |
| | | YADSVKGRFTISSDNAKNTVFLQMNSLKP |
| | | EDTAVYYCNKAPPFTTIQPMCVMIDTQLG |
| | | AWGQGTQVTVSS |
| VHH9 | SEQ ID NO: 9 | DVQLQESGGGLVQAGGSLRLSCAASGRGG |
| | | ASYAMGWFRQAPGKEREFVAAISWTGDTT |
| | | YNVDSVKGRFTISSDNAKNPVYLDMSSLK |
| | | PEDTAVYYCAAAHWTRVVAKGAMADEKK |
| | | YEYWGQGTQVTVSS |
| VHH10 | SEQ ID NO: 10 | DVQLQESGGGSVQAGGSLRLSCAASGRTFS |
| | | SNFMGWFRQAPNKDREFVAAIHWSGGSRAY |
| | | ADSVKGRFTISRDNTKDTVYLLVNSLKLE |
| | | DTAVYYCAAEISWQTTGWTNLGSWGQGT |
| | | QVTVSS |
| VHH11 | SEQ ID NO: 11 | DVQLQESGGGLVQAGGSLRLSCAASGRTF |
| | | SSDFMGWFRQAPGKEREFVAAIHWSGGST |
| | | SYADSVKGRFTISRDNAKDTVYLLMNSLK |
| | | LEDTAVYYCAAEIRWLTTGWANLGSWGQ |
| | | GTQVTVSS |
| VHH12 | SEQ ID NO: 12 | DVQLQESGGGLVQAGGSLRLSCAASGRTF |
| | | SSDFMGWFRQAPGKEREFVAAIHWSGGSR |
| | | SYADSVKGRFTISRDNAKDTVYLLMNSLK |
| | | LEDTAVYYCAAEIRWQTTGWANLGSWGQ |
| | | GTQVTVSS |
| VHH13 | SEQ ID NO: 13 | DVQLQESGGGLAQPGGPLRLSCAASGRTFT |
| | | SDFMGWFRQAPGKEREFVAAIHWSGGSRS |
| | | YADSVKGRFTISRDNAKDTVYLLMNSLKL |
| | | EDTAVYYCAAEIRWQTAGWVNLGSWGQG |
| | | TQVTVSS |
| VHH14 | SEQ ID NO: 14 | DVQLQESGGGLVQPGGSLRLSCEASGVTF |
| | | NFYSMTWYRQAPGKERELVADITSEGRTE |
| | | YAHSVKGRFIISRDDFGTMVYLQMNSLKP |
| | | EDTAVYYCYTGGSWGQGTQVTVSS |
| VHH15 | | DVQLQESGGGLVQAGGSLRLSCAVSGSTF |
| | | RLNVMGWYRQAPGKLRDLVAGITADGGV |
| | SEQ ID NO: 15 | TNYADSVKGRFIISRDNAKNTVYLQMNSL |
| | | KPEDTAIYYCNTAPANWGSWGQGTQVTVS |
| | | S |
| VHH16 | SEQ ID NO: 16 | DVQLQESGGGLVQPGGSLRLSCTASGSIF |
| | | SSLTMAWWRQAPGKQRELVAGITTDGSTD |
| | | YADSVKGRFTISRDNAKNTVYLQMDSLKP |
| | | EDTAVYYCNKSPYYDPTDYDSWGQGTQVT |
| | | VSS |

7. The compound according to claim 6, wherein said VHH antibody domain comprises the VHH sequence VHH1 shown in the Table of VHH Sequences or a fragment thereof, with additional modification of (B) or (C), wherein said modification of (B) or (C) consists of the substitution of one or more amino acids compared to the sequence of SEQ ID NO: 1, wherein said substitution is at least one selected from the group consisting of D1E, Q5V, L11V, T24A, E44G, H45L, G47W, F70I, V79L, K87R, P88A, and Q116L.

8. The compound according to claim 6, wherein said VHH antibody domain comprises a VHH sequence represented by at least one SEQ ID NO selected from the group consisting of SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 77, and a fragment thereof.

9. The compound according to claim 6, wherein said VHH antibody domain comprises the VHH sequence VHH1 shown in the Table of VHH Sequences or a fragment thereof, with additional modification of (B) or (C), wherein said modification of (B) or (C) consists of the substitution of one or more amino acids compared to the sequence of SEQ ID NO: 2, wherein said substitution is at least one selected from the group consisting of D1E, Q5V, L11V, A14P, V24A, F37V, A44G, R45L, S47W, V79L, K87R, P88A, and Q118L.

10. The compound according to claim 6, wherein said VHH antibody domain comprises a VHH sequence represented by at least one SEQ ID NO selected from the group consisting of SEQ ID NO: 79, SEQ ID NO: 80, SEQ ID NO: 81, SEQ ID NO: 83, SEQ ID NO: 84, SEQ ID NO: 85, SEQ ID NO: 86, SEQ ID NO: 89, SEQ ID NO: 90, SEQ ID NO: 93, SEQ ID NO: 94, and a fragment thereof.

11. The compound according to claim 1, wherein said compound binds to NKp30 with an affinity of at least about 1×10⁶ M⁻¹ in a binding assay by BLI (biolayer interferometry) with recombinant extracellular domain of NKp30 (NKp30-ECD, SEQ ID NO: 66) in KB buffer (PBS+0.1% Tween-20+1% BSA).

12. The compound according to claim 1, wherein said compound further comprises a targeting moiety.

13. The compound according to claim 12, wherein said targeting moiety is capable of specifically binding to a tumor-associated antigen.

14. The compound according to claim 1, wherein said compound comprises an antibody Fc region competent in Fc receptor binding.

15. A pharmaceutical composition, comprising: the compound according to claim 1.

16. The compound according to claim 1, wherein the compound is a medicament.
